(19) United States
(12) Patent Application Publication
(10) Pub. No.: US 2026/0183298 A1
(43) Pub. Date: Jul. 2, 2026

(54) VIRAL PROPHYLAXIS TREATMENT METHODS AND PRE-EXPOSURE PROPHYLAXIS KITS

(71) Applicant: Christina M. RAMIREZ, Monrovia, CA (US)

(72) Inventor: Christina M. RAMIREZ, Monrovia, CA (US)

(21) Appl. No.: 19/125,955

(22) PCT Filed: Oct. 31, 2023
(86) PCT No.: PCT/US2023/036357

§ 371 Date: Apr. 30, 2025

Related U.S. Application Data
(60) Provisional application No. 63/381,839, filed on Nov. 1, 2022.
(60) Provisional application No. 63/579,629, filed on Aug. 30, 2023.

(51) Int. Cl. A61K 31/522; A61K 31/403; A61K 31/4245; A61K 31/513; A61K 31/52; A61K 31/7056; A61K 38/21; A61P 31/22

(52) U.S. Cl. CPC A61K 31/522; A61K 31/403; A61K 31/4245; A61K 31/513; A61K 31/52; A61K 31/7056; A61K 38/21; A61P 31/22

(57) ABSTRACT

The present disclosure provides compositions and methods for the prevention of herpesvirus infection in a herpesvirus seronegative subject and for the treatment and prevention of recurrence in a herpesvirus seropositive subject.

### RELATED APPLICATIONS AND INCORPORATION BY REFERENCE

This application claims benefit of priority to US provisional patent application Serial Nos. 63/381,839 filed Nov. 1, 2022 and 63/579,629 filed Aug. 30, 2023.

All documents cited herein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The invention relates generally to a combination of compounds with antiviral activity and, more specifically, with anti-herpes properties. This invention provides a composition of matter and a method of use for preventing and treating herpesvirus infections, which, in turn, can cause other disease states.

### BACKGROUND OF THE INVENTION

The hallmark of alpha, beta, and gamma human herpesviruses is that they all establish latent infections. These viral infections have widespread, worldwide prevalence. Consequences of these infections range from asymptomatic disease (i.e., seropositivity) to repeated and/or severe episodes of symptoms caused by the active form of the disease. Additionally, there are many herpesviruses that infect non-human animals and like the herpesviruses of humans, herpesviruses of animals cause severe diseases that influence not only the health and epidemiological status, but are also economically important in the context of food production. Additionally, the members of Herpesviridae are host to specific agents that also share many properties and potentially make them capable of crossing the species barrier.

As in herpesvirus infections in humans, herpesvirus infection in non-human animals is lifelong. Classic nucleoside analogs are useful for the treatment of herpesvirus related diseases in humans such as HSV-1 and HSV-2, as well as varicella zoster virus (i.e., VZV), and, to a lesser extent, Epstein Barr virus (i.e., EBV) and cytomegalovirus (i.e., CMV). A nucleoside analog that is useful in the treatment of HSV-1 and HSV-2, valacyclovir, has been used to treat other herpesvirus infections with mixed results.

Once humans and/or non-human animals are infected, the herpesvirus establishes a latent infection that is not cleared by the immune system even with the help of vaccines or antivirals. It is thought that infected humans and non-human animals can undergo virus reactivation many times including in an asymptomatic manner and spread the virus widely.

While most care for herpesvirus infection is supportive, single antiviral therapy using valacyclovir has been reported with mixed results in its ability to prevent herpesvirus infection. This single-agent approach exacerbates the single point of failure problem, as cumulative effects of multiple agents are not realized. In addition, a viral strain may evolve and develop resistance to the single agent in use, weakening or even possibly neutralizing the single layer protection.

Valacyclovir, prescribed and administered as a single-dose treatment (i.e., monotherapy), has been utilized in humans and to a lesser extent non-human animals and has shown to reduce the level of viremia but has had mixed results in the prevention of herpesvirus infection.

The use of antiviral agents in novel combinations can yield an increase in agent efficacy, or possibly, an equivalent antiviral effect, but with reduced dosage, thereby potentially reducing or limiting risks related to agent toxicity.

Citation or identification of any document in this application is not an admission that such document is available as prior art to the present invention.

### SUMMARY OF THE INVENTION

In one aspect, the invention provides methods and compositions of matter for preventing and treating herpesvirus infection.

In some embodiments, the invention provides a pharmaceutical composition for treatment of herpesvirus infection, prophylaxis, or pre-exposure prophylaxis against herpesvirus infection in a subject in need thereof comprising: a therapeutically effective amount of (a) a first antiviral agent or a salt or solvate thereof and (b) a second antiviral agent or a salt or solvate thereof; and pharmaceutically acceptable excipient(s), diluent(s), carrier(s), and/or adjuvant(s).

In some embodiments, the pharmaceutical composition comprises (a) and (b) in a dose ratio ((a):(b)) in mg/kg of about 5:1, about 4:1, about 3:1, about 2:1, about 1:1, about 1:2, about 1:3, about 1:4, or about 1:5.

In some embodiments, the pharmaceutical composition comprises 0.01 mg/kg body weight to about 1000 mg/kg body weight of (a) and 0.01 mg/kg body weight to about 1000 mg/kg body weight of (b).

In some embodiments, the pharmaceutical composition comprises about 1 mg/kg body weight to about 500 mg/kg body weight of (a).

In some embodiments, the pharmaceutical composition comprises about 500 mg/kg body weight to about 1000 mg/kg body weight of (a).

In some embodiments, the pharmaceutical composition comprises about 1 mg/kg body weight to about 500 mg/kg body weight of (b).

In some embodiments, the pharmaceutical composition comprises about 500 mg/kg body weight to about 1000 my/kg body weight of (b).

In some embodiments, the pharmaceutical composition is formulated for oral administration.

In some embodiments, the oral formulation comprises a tablet, capsule, soft chew, a gel, a paste, a powder, a bait drop or a liquid.

In some embodiments, the pharmaceutical composition is formulated for parenteral administration.

In some embodiments, the parenteral administration comprises an intravenous injection or infusion, an intramuscular injection, or a subcutaneous injection.

In some embodiments, the parenteral administration comprises an intravenous injection or infusion.

In some embodiments, the pharmaceutical composition is formulated for administration once, twice, thrice, or four times daily.

In some embodiments, the pharmaceutical composition is formulated for long-acting administration.

In some embodiments, the present invention provides the pharmaceutical composition, wherein (a) a first antiviral agent or a salt or solvate thereof comprises valacyclovir or a salt or solvate thereof, and wherein (b) a second antiviral agent or a salt or solvate thereof comprises famciclovir or a salt or solvate thereof.

In some embodiments, the pharmaceutical composition additionally comprises a therapeutically effective amount of an additional antiviral agent.

In some embodiments, the additional antiviral agent comprises a helicase-primase inhibitor, amenamevir, N-methancocarbathymidine (N-MCT), tenofovir, emtricitabine, lamivudine, interfereon, ribavirin, boceprevir, telaprevir, simeprevir, sofosbuvir, ledipasvir, tenofovir, ombitasvir, paritaprevir, penciclovir, pritelivir, brincidofovir, cidofovir, ganciclovir, valganciclovir, valomaciclovir, or ritonavir, C5A.

In some embodiments, the herpesvirus of the present invention is a member of the Equine herpesvirus, Gallid herpesvirus, or Bovine herpesvirus family of herpesviruses.

In some embodiments, the herpesvirus comprises a member of the Equine herpesvirus family of herpesviruses.

In some embodiments, the Equine herpesvirus comprises EHV-1, EHV-3, EHV-4, or EHV-5.

In some embodiments, the herpesvirus is a member of the Gallid herpesvirus family of herpesviruses.

In some embodiments, the Gallid herpesvirus comprises GaAHV1 or GaAHV2.

In some embodiments, the herpesvirus is a member of the Bovine family of herpesviruses.

In some embodiments, the Bovine herpesvirus comprises BoAHV1 or BoAHV5.

The present invention also envisions, a pharmaceutical composition for treatment of Equine Herpesvirus (EHV), Gallid herpesvirus (GHV), or Bovine herpesvirus (BHV) infection, prophylaxis, or pre-exposure prophylaxis against EHV, GHV, or BHV infection in a subject in need thereof comprising: a therapeutically effective amount of greater than 0.5 mg/kg body weight of one or more antiviral agent(s).

In some embodiments, the present invention also provides the pharmaceutical composition, wherein the amount of the one or more antiviral agent(s) is from greater than 1 mg/kg to about 1000 mg/kg body weight.

In some embodiments, the present invention also provides the pharmaceutical composition, wherein the one or more antiviral agent(s) comprises valacyclovir or a salt or solvate thereof, famciclovir or a salt or solvate thereof, penciclovir or a salt or solvate thereof, acyclovir or a salt or solvate thereof, or any combination thereof.

In some embodiments, the pharmaceutical composition comprises valacyclovir or a salt or solvate thereof and famciclovir or a salt or solvate thereof.

In some embodiments, the pharmaceutical composition comprises about 0.05 mg to about 1000 mg/kg body weight of valacyclovir or a salt or solvate thereof and about 0.05 mg/kg to about 1000 mg/kg bodyweight of famciclovir or a salt or solvate thereof.

In some embodiments, the pharmaceutical composition comprises valacyclovir or a salt or solvate thereof and acyclovir or a salt or solvate thereof.

In some embodiments, the pharmaceutical composition comprises about 0.05 mg to about 1000 mg/kg body weight of valacyclovir or a salt or solvate thereof and about 0.05 mg/kg to about 1000 mg/kg bodyweight of acyclovir or a salt or solvate thereof.

In some embodiments, the pharmaceutical composition comprises valacyclovir or a salt or solvate thereof and penciclovir or a salt or solvate thereof.

In some embodiments, the pharmaceutical composition comprises about 0.05 mg to about 1000 mg/kg body weight of valacyclovir or a salt or solvate thereof and about 0.05 mg/kg to about 1000 mg/kg bodyweight of penciclovir or a salt or solvate thereof.

In some embodiments, the pharmaceutical composition is formulated for oral administration.

In some embodiments, the pharmaceutical composition comprises a tablet, capsule, soft chew, a gel, a paste, a powder, a bait drop, or a liquid.

In some embodiments, the pharmaceutical composition is formulated for parenteral administration.

In some embodiments, the parenteral administration comprises an intravenous injection or infusion, an intramuscular injection, or a subcutaneous injection.

In some embodiments, the parenteral administration comprises an intravenous injection or infusion.

In some embodiments, the pharmaceutical composition is formulated for administration once, twice, thrice, or four times daily.

In some embodiments, the pharmaceutical composition is formulated for long-acting administration.

In some embodiments, the pharmaceutical composition additionally comprises a therapeutically effective amount of an additional antiviral agent.

In some embodiments, the additional antiviral agent comprises a helicase-primase inhibitor, amenamevir, N-methancocarbathymidine (N-MCT), tenofovir, emtricitabine, lamivudine, interfereon, ribavirin, boceprevir, telaprevir, simeprevir, sofosbuvir, ledipasvir, tenofovir, ombitasvir, paritaprevir, penciclovir, pritelivir, brincidofovir, cidofovir, ganciclovir, valganciclovir, valomaciclovir, or ritonavir.

In some embodiments, the herpesvirus of the present invention is a member of the Equine herpesvirus, Gallid herpesvirus or Bovine herpesvirus family of herpesviruses.

In some embodiments, the herpesvirus comprises a member of the Equine herpesvirus family of herpesviruses.

In some embodiments, the Equine herpesvirus comprises EHV-1, EHV-3, EHV-4, or EHV-5.

In some embodiments, the herpesvirus comprises a member of the Gallid herpesvirus family of herpesviruses.

In some embodiments, the Gallid herpesvirus comprises GaAHV1 or GaAHV2.

In some embodiments, the herpesvirus comprises a member of the Bovine herpesvirus family of herpesviruses.

In some embodiments, the Bovine herpesvirus comprises BoAHV1 or BoAHV5.

The present invention also envisions, a method of pre-exposure prophylaxis against EHV, GHV, or BHV infection in a herpesvirus seronegative subject in need thereof, or a method of treating a herpesvirus infection, or a method of suppressing a herpesvirus infection, or a method of treatment or prophylactic treatment of a disease or condition associated with EHV, GHV, or BHV infection, the method comprising administering to the subject daily a therapeutically effective amount of (a) a first antiviral or a salt or solvate thereof and (b) a second antiviral or a salt or solvate thereof; and pharmaceutically acceptable excipient(s), diluent(s), carrier(s) and/or adjuvant(s).

In some embodiments, the pharmaceutical composition comprises (a) and (b) in a dose ratio in mg/kg of about 5:1, about 4:1, about 3:1, about 2:1, about 1:1, about 1:2, about 1:3, about 1:4, or about 1:5.

In some embodiments, the pharmaceutical composition comprises 0.05 mg/kg body weight to about 1000 mg/kg body weight of (a) and 0.05 mg/kg body weight to about 1000 mg/kg body weight of (b).

In some embodiments, the disease or infection associated with EHV infection is Equine Myeloencephalopathy (EHM) or Equine Multinodal Pulmonary Fibrosis (EMPF).

In some embodiments, the disease or infection associated with GHV infection is infectious laryngotraceitis (ILT) or Marek's Disease.

In some embodiments, the disease or infection associated with BHV infection is bovine rhinotracheitis or bovine meningoencephalitis.

The present invention also envisions, a method of pre-exposure prophylaxis against Equine Herpesvirus (EHV), Gallid Herpesvirus (GHV), or Bovine Herpesvirus (BHV) infection in a herpesvirus seronegative subject in need thereof, or a method of treating a herpesvirus infection, or a method of suppressing a herpesvirus infection, or a method of treatment or prophylactic treatment of a disease or condition associated with EHV, GHV, or BHV infection, the method comprising administering to the subject daily a pharmaceutical composition comprising a therapeutically effective amount of greater than 0.5 mg/kg body weight of an antiviral agent(s) comprising acyclovir or a salt or solvate thereof, or valacyclovir or a salt or solvate thereof, penciclovir or a salt or solvate thereof, famciclovir or a salt or solvate thereof, or combinations thereof, whereby the risk of EHV, GHVm or BHV infection in the subject is reduced.

In some embodiments, the pharmaceutical composition comprises about 0.5 mg/kg body weight to about 1000 mg/kg body weight of the antiviral agent(s).

In some embodiments, the antiviral agent(s) comprises valacyclovir or a salt or solvate thereof.

In some embodiments, the antiviral agent(s) comprises famciclovir or a salt or solvate thereof.

In some embodiments, the antiviral agent(s) comprises penciclovir or a salt or solvate thereof.

In some embodiments, the antiviral agent(s) comprises acyclovir or a salt or solvate thereof.

In some embodiments, the method comprises administering 0.5 mg/kg to about 1000 mg/kg body weight of a second antiviral agent.

In some embodiments, the antiviral agent(s) comprises valacyclovir or a salt or solvate thereof and famciclovir or a salt or solvate thereof.

In some embodiments, the antiviral agent(s) comprises valacyclovir or a salt or solvate thereof and acyclovir or a salt or solvate thereof.

In some embodiments, the antiviral agent(s) comprise valacyclovir or a salt or solvate thereof and penciclovir or a salt or solvate thereof.

In some embodiments, The method comprises administering about 0.05 mg/kg body weight to about 1000 mg/kg body weight of valacyclovir or a salt or solvate thereof and about 0.05 mg/kg body weight to about 1000 mg/kg body weight of famciclovir or a salt or solvate thereof.

In some embodiments, the administering is oral.

In some embodiments, the administering is parenteral.

In some embodiments, the parenteral administration is intravenous.

In some embodiments, the administering is once daily, twice daily, thrice daily, or four times daily.

In some embodiments, the administering begins after exposure to a subject that is seropositive for herpesvirus.

In some embodiments, the administering begins prior to exposure to a subject that is seropositive to herpesvirus.

In some embodiments, the method comprises administering the pharmaceutical composition to the herpesvirus seropositive subject.

In some embodiments, the herpesvirus is a member of the Equine herpesvirus, Gallid herpesvirus, or Bovine herpesvirus family of herpesviruses.

In some embodiments, the herpesvirus comprises a member of the Equine herpesvirus family of herpesviruses.

In some embodiments, the Equine herpesvirus comprises EHV-1, EHV-3, EHV-4, or EHV-5.

In some embodiments, the herpesvirus comprises a member of the Gallid herpesvirus family of herpesviruses.

In some embodiments, the Gallid herpesvirus comprises GaAHV1 or GaAHV2.

In some embodiments, the herpesvirus comprises a member of the Bovine herpesvirus family of herpesviruses.

In some embodiments, the Bovine herpesvirus comprises BoAHV1 or BoAHV5.

In some embodiments, the disease or infection associated with EHV infection is Equine Myeloencephalopathy (EHM) or Equine Multinodal Pulmonary Fibrosis (EMPF).

In some embodiments, the disease or infection associated with GHV infection is infectious laryngotracheitis (ILT) or Marek's Disease.

In some embodiments, the disease or infection associated with BHV infection is bovine rhinotracheitis or bovine meningoencephalitis.

### DETAILED DESCRIPTION OF THE INVENTION

The current standard practice for preventing herpesvirus infection involves supportive care of a herpesvirus seropositive subject to reduce transmission of the virus to a herpesvirus seronegative subject. As described previously, these methods rely on the compliance of a herpesvirus seropositive subject and often do not provide adequate protection for the herpesvirus seronegative subject. Described herein, in various aspects, are methods for the prevention of herpesvirus infection in a seronegative subject comprising administering to the seronegative subject a composition comprising one or more antiviral agents.

The term “herpesvirus” includes all herpesviruses, including but not limited to Gallid alphaherpesvirus 1 (GaAHV1), Psittacid alphaherpesvirus 1 (PsAHV1), Anatid alphaherpesvirus 1 (AnAHV1), Columbid alphaherpesvirus 1 (CoAHV1), Gallid alphaherpesvirus 2 (GaAHV2), Gallid alphaherpesvirus 3 (GaAHV3), Meleagrid alphaherpesvirus 1 (MeAHV1), Spheniscid alphaherpesvirus 1 (SpAHV1), Chelonid alphaherpesvirus 5 (ChAHV5), Testudinid alphaherpesvirus 3 (TeAHV3), Ateline alphaherpesvirus 1 (AtAHV1), Bovine alphaherpesvirus 2 (BoAHV2), Cercopithecine alphaherpesvirus 2 (CeAHV2), Human alphaherpesvirus 1 (HuAHV1), Human alphaherpesvirus 2 (HuAHV2), Leporid alphaherpesvirus 4 (LeAHV4), Macacine alphaherpesvirus 1 (McAHV1), Macacine alphaherpesvirus 2 (McAHV2), Macropodid alphaherpesvirus 1 (MaAHV1), Macropodid alphaherpesvirus 2 (MaAHV2), Panine alphaherpesvirus 3 (PnAHV3), Papiine alphaherpesvirus 2 (PaAHV2), Pteropodid alphaherpesvirus 1 (PtHAV1), Saimiriine alphaherpesvirus 1 (SaAHV1), Bovine alphaherpesvirus 1 (BoAHV1), Bovine alphaherpesvirus 5 (BoAHV5), Bubaline alphaherpesvirus 1 (BuAHV1), Canid alphaherpesvirus 1 (CaAHV1), Caprine alphaherpesvirus 1 (CpAHV1), Cercopithecine alphaherpesvirus 9 (CeAHV9), Cervid alphaherpesvirus 1 (CvAHV1), Cervid alphaherpesvirus 2 (CvAHV2), Cerbid alphaherpesvirus 3 (CvAHV3), Equid alphaherpesvirus 1 (EqAHV1, EHV-1), Equid alphaherpesvirus 3 (EqAHV3, EHV-3), Equid alphaherpesvirus 4 (EqAHV4, EHV-4), Equid alphaherpesvirus 8 (EqAHV8, EHV-8), Equid alphaherpesvirus 9 (EqAHV9, EHV-9), Felid alphaherpesvirus 1 (FeAHV1), Human alphaherpesvirus 3 (HuAHV3), Monodontid alphaherpesvirus 1 (MoAHV1), Phocid alphaherpesvirus 1 (PcAHV1), Suid alphaherpesvirus 1 (SuAHV1), Chelonid alphaherpesvirus 6 (ChAHV6), Aotine betaherpesvirus 1 (AoBHV1), Cebine betaherpesvirus 1 (CbBHV1), Cercopithecine betaherpesvirus 5 (CeBHV5), Human betaherpesvirus 5 (HuBHV5), Macacine betaherpesvirus 3 (McBHV3), Macacine betaherpesvirus 8 (McBHV8), Mandrille betaherpesvirus 1 (MdBHV1), Panine betaherpesvirus 2 (PnBHV2), Papiine betaherpesvirus 4 (PaBHV4), Saimiriine betaherpesvirus 4 (SaBHV4), Murid betaherpesvirus 1 (MuBHV1), Murid betaherpesvirus 2 (MuBHV2), Murid betaherpesvirus 8 (MuBHV8), Elephantid betaherpesvirus 1 (E1BHV1), Elephantid betaherpesvirus 4 (E1BHV4), Elephantid betaherpesvirus 5 (E1BHV5), Caviid betaherpesvirus 2 (CdBHV2), Miniopterid betaherpesvirus 1 (MnBHV1), Tupaiid betaherpesvirus 1 (TuBHV1), Human betaherpesvirus 7 (HuHBV7), Human betaherpesvirus 6A (HuBHV6A), Human betaherpesvirus 6B (HuBHV6B), Macacine betaherpesvirus 9 (McBHV9), Murid betaherpesvirus 3 (MuHBV3), Suid betaherpesvirus 2 (SuBHV2), Dephinid gammaherpesvirus 1 (DeGHV1), Callitrichine gammaherpesvirus 3 (C1GHV3), Cercopithecine gammaherpesvirus 14 (CeGHV14), Gorilline gammaherpesvirus 1 (GoGHV1), Human gammaherpesvirus 4 (HuGHV4), Macacine gammaherpesvirus 4 (McGHV4), Macacine gammaherpesvirus 10 (McGHV10), Panine gammherpesvirus 1 (PnGHV1), Papiine gammaherpesvirus 1 (PaGHV1), Pongine gammaherpesvirus 2 (PoGHV2), Alcelaphine gammaherpesvirus 1 (ALGHV1), Alcelaphine gammaherpesvirus 2 (AlGHV2), Bovine gammaherpesvirus 6 (BoGHV6), Caprine gammaherpesvirus 2 (CpGHV2), Hippotragine gammaherpesvirus 1 (HiGHV1), Ovine gammaherpesvirus 2 (OvGHV2), Suid gammaherpesvirus 3 (SuGHV3), Suid gammaherpesvirus 4 (SuGHV4), Suid gammaherpesvirus 5 (SuGHV5), Phascolarctid gammaherpesvirus 1 (PcGHV1), Vombatid gammaherpesvirus 1 (VoGHV1), Vespertilionid gammaherpesvirus 3 (VeGHV3), Equid gammaherpesvirus 2 (EqGHV2, EHV-2), Equid gammaherpesvirus 5 (EqGHV5, EHV-5), Felid gammaherpesvirus 1 (FeGHV1), Mustelid gammaherpesvirus 1 (MusGHV1), Phocid gammaherpesvirus 3 (PhGHV3), Verspertilionid gammaherpesvirus 1 (VeGHV1), Ateline gammaherpesvirus 2 (AtGHV2), Ateline gammaherpesvirus 3 (AtGHV3), Bovine gammaherpesvirus 4 (BoGHV4), Cricetid gammaherpesvirus 2 (CrGHV2), Human gammaherpesvirus 8 (HuGHV8), Macacine gammaherpesvirus 5 (McGHV5), Macacine gammaherpesvirus 8 (McGHV8), Macacine gammaherpesvirus 11 (McGHV11), Macacine gammaherpesvirus 12 (McGHV12), Murid gammaherpesvirus 4 (MuGHV4), Murid gammaherpesvirus 7 (MuGHV7), Saimiriine gammaherpesvirus 2 (SaGHV2), Equid gammaherpesvirus 7 (EGHV7, EHV-7), Phocid gammaherpesvirus 2 (PhoGHV2), Saguinine gammaherpesvirus 1 (SgGHV1), Iguanid herpesvirus 2 (IgHV2), Anguillid herpesvirus 1 (AngHV1), Cyprinid herpesvirus 1 (CyHV1), Cyprinid herpesvirus 2 (CyHV2), Cyprinid herpesvirus 3 (CyHV3), Ictalurid herpesvirus 1 (IcHV1), Ictalurid herpesvirus 2 (IcHV2), Acipenserid herpesvirus 1 (AciHV1), Acipenserid herpesvirus 2 (AciHV2), Salmonid herpesvirus 1 (SalHV1), Salmonid herpesvirus 2 (SalHV2), Salmonid herpesvirus 3 (SalHV3), Gadid herpesvirus 1 (GaHV1), Ranid herpesvirus 1 (RaHV1), Ranid herpesvirus 2 (RaHV2), Pilchard herpesvirus, Tilapia herpesvirus, Percid herpesvirus 1 (PeHV1), Emydoidea herpesvirus 1 (EBHV-1), Pelomedusid herpesvirus 1, Glyptemys herpesvirus 1 (GlyHV-1), Glyptemys herpesvirus 2 (GlyHV-2), Emydid herpesvirus 1 (EmyHV-1), Emydid herpesvirus 2 (EmyHV-2), Terrapene herpesvirus 1 (TerHV-1), and Terrapene herpesvirus 2 (TerHV-2).

In many aspects of the disclosure, described herein are compositions and methods for the prevention and treatment of a herpesvirus infection in a seronegative or seropositive subject. Non-limiting examples of the seropositive or seronegative subject include production animals, companion animals, and/or animals held in captivity (for example, zoo animals or lab animals) which include, but are not limited to, mammals, birds, fish, amphibians, and reptiles. Non-limiting examples of mammals include mammals belonging to the orders Rodentia, Chiroptera, Soricomorpha, Primates, Carnivora, Artiodactyla, Diprotontia, Lagomorpha, Didelphimorphia, Cetacea, Dasyuromorphia, Afrosoricida, Erinaceomorpha, Cingulata, Peramelemorphia, Scandentia, Perissodactyla, Macroscelidea, Pilosa, Monotremeta, Sirenia, and Proboscidea. Non-limiting examples of mammals, birds, fish, amphibians, and reptiles include horses, donkeys, cattle, zebu, yaks, water buffalo, gayal, sheep, goat, reindeer, camels, llamas, alpacas, pigs, boar, chickens, rabbits, guinea pigs, duck, pigeons, geese, guineafowl, doves, turkey, ostrich, banteng, mink, domestic cats, large cats, dogs, wolves, aardvarks, elephants, parrots, alligators, fish, bison, iguanas, foxes, badgers, eagles, snakes, lizards, bears, rats, mice, rhinoceros, skunks, skinks, bluebirds, canaries, buffalo, frogs, turtles, tortoise, newts, salamanders, toads, owls, chameleon, chimpanzee, chinchilla, chipmunk, hamsters, penguin, emu, echidna, elk, shrews, falcons, ferrets, flamingos, squirrels, gazelle, gerbil, bats, giraffe, tigers, lions, cheetahs, jaguars, puma, groundhog, grouse, finches, pheasant, hawk, hedgehog, monkeys, impala, kangaroo, deer, koala, koi, lemur, leopard, lynx, macaque, macaw, mandrill, mole, mongoose, moose, oxen, heron, osprey, orangutan, gorilla, opossum, ocelot, peacock, platypus, porcupine, possum, proboscis monkey, quetzal, raccoon, pandas, otters, sloth, swan, tapir, toucan, weasel, and zebra.

As used herein, the term “companion animal” refers to any animal for which an owner, breeder or caregiver controls the feeding habits and/or the animal is kept as a pet. Non-limiting examples of companion animals include dog, cat, rabbit, hamster, gerbil, ferret, and guinea pig.

As used herein, the term “production animal” or “livestock” refers to animals that are usually raised for food or a product that can be sold (such as wool, milk, eggs, etc.), such as cattle, sheep, goats, birds (chickens, turkeys), pigs, buffalo and fish.

As used herein, the term “animals held in captivity” refers to animals that are under the care of zoos, aquariums, wildlife refuges, national parks, animal shelters, animal sanctuaries, and animal management/care facilities.

In many aspects of the disclosure, the compositions and methods described herein are used to prevent and treat a variety of diseases and disorders caused by herpesvirus infection. Non-limiting examples of diseases and/or disorders caused by herpesvirus infections include infectious laryngotracheitis, Pacheco's diseases, duck viral enteritis, Marek's disease, infectious bovine rhinotracheitis, bovine encephalitis, equine coital exanthema, equine rhinopneumonitis, equine herpes Myeloencephalopathy, feline viral rhinotracheitis, pseudorabies, lung-eye-trachea disease, fibropapillomatosis, mononucleosis, pneumonia, acute hemorrhagic disease, lymphosarcoma, malignant catarrhal fever, Kaposi's sarcoma, retroperitoneal fibromatosis, Amazon tracheitis, equine multinodular pulmonary fibrosis and Aujeszky's disease.

In some aspects of the present disclosure, the compositions and methods described herein are used to prevent and treat herpesvirus infections in equines and camelids. Non-limiting examples of herpesviruses that may infect equines and camelids include EHV-1, EHV-2, EHV-3, EHV-4, EHV-5, EHV-6, EHV-7, EHV-8, and EHV-9.

In some embodiments, the compositions and methods described herein are used to prevent or treat equine herpes Myeloencephalopathy. Equine herpes Myeloencephalopathy is a neurological disorder caused by EHV-1 and EHV-4 in equines and camelids and have especially have been shown to cause abortions. Equine Herpesviruses are a contagious virus that easily spreads among equines and camelids and typically causes mild respiratory symptoms and occasionally neurological symptoms, newborn death, and abortion. EHM is often fatal and survivors face neurological problems. Once an equine or camelid is positive for EHM, strict biosecurity measures take place and equines and camelids are isolated for 21 days to reduce the spread of the disease. Once infected, disease signs usually manifest within 4 to 6 days after exposure. Fever is often the first indication and temperatures over 101.5° F. are indicative of infection. Other signs include coughing, and thick, clear to yellow, nasal discharge. Neurological disease typically manifests 8-12 days after the onset of the fever and includes hind-end weakness, loss of balance, and inability to stand. The virus can spread by direct subject-to-subject contact as well as via airborne droplets. Additionally, the virus can live on contaminated surfaces for an extended period. Therefore, EHV can also spread through contact with infected objects (formite transmission), such as saddles, tack, feed, and waterbuckets, as well as, human hands and clothing. Clinical treatment of EHV is typically a combination of supportive and palliative care, such as supportive slings to assist equines and camelids who have an inability to stand, and anti-inflammatory agents. Antiviral medications, such as valacyclovir, have had mixed results. While vaccinations are approved to help control the respiratory symptoms of EHV, they are not licensed to prevent EHM. Though there are several inactivated and modified live vaccines for EHV (Duvaxyn® EHV-1,4, PRESTIGE® vaccine line, and Vetera® vaccine line), to date, none are approved for the prevention of EHM. Despite EHV vaccination efforts, the incidence of EHM outbreaks has been increasing over the years. There is an unmet need for treatment/prevention for EHM as the current standard of care is palliative and the fatality rate of EHM is up to 50%. Furthermore, EHV outbreaks have been increasing despite the widespread vaccination effort. Novel variants are becoming known and three mutations have been classified, D752, N752, and H752. The D752 variant causes higher levels of viremia and is more associated with neurological disease. Cases of the neurological form of EHV (EHM) are increasing since its first declaration as an emerging disease in the US in 2007. EHV infects most equines and camelids by the time they are adults and most never exhibit any symptoms. However, stress events such as shipping, can cause the virus to reactivate and spread through normal respiration, even in an asymptomatic manner. A 2021 outbreak in a horse show barn in Pennsylvania highlights this fact. In this outbreak, about 75% of the horses became infected with EHV despite being up-to-date on their EHV vaccinations. All of the horses were treated with valacyclovir and none progressed to severe neurological disease. However, two horses that were temporally and epidemiologically associated with the outbreak, but from different locations and untreated with valacyclovir had to be euthanized.

In some embodiments, the compositions and methods described herein are used to prevent or treat equine multinodular pulmonary fibrosis (EMPF). EHV-5 is known to cause EMPF in horses. EMPF is a chronic lung disease of horses that effects adult horses ability to exercise as a result of the formation of nodular lesions in the lungs. EMPF is characterized by weight loss, fever, respiratory distress and depression, unwillingness to move, mild cough, and intermittent tachypnea. Currently, there is no treatment or specific preventative measures for EMPF.

In some embodiments, the compositions and methods described herein are used to prevent or treat equine coital exanthema. EHV-3 is known to cause equine coital exanthema in horses. Equine coital exanthema is a venereal disease that affects both sexes and is characterized by the appearance of multiple, circular, red nodules up to 2 mm in diameter on the vulvar and vaginal mucosa, the clitoral sinus, perineal skin, penis, and prepuce that eventually develop into shallow, painful, ulcers. Currently, there is no treatment and preventative measures are limited to sexual rest until ulcers are healed.

In some embodiments, the compositions and methods described herein are used to prevent or treat equine viral rhinopneumonitis. EHV-1 and EHV-4 are known to cause equine viral rhinopneumonitis in equines and camelids. Equine viral rhinopneumonitis is a respiratory disease that is characterized by fever, nasal discharge, depression, throat inflammation, cough, poor appetite, and enlarged lymph nodes. As the disease progresses, it may lead to paralysis of the hind end, loss of bladder and tail function, and death. There currently is no treatment available and vaccinations are not very accessible and provide only limited immunity.

In some aspects of the present disclosure, the compositions and methods described herein are used to prevent and treat herpesvirus infections in birds. Non-limiting examples of herpesviruses that may infect birds include Gallid alphaherpesvirus 1, Psittaeid alphaherpesvirus 1, Anatid alphaherpesvirus 1, Columbid alphaherpesvirus 1, Gallid alphaherpesvirus 2, Gallid alphaherpesvirus 3, Meleagrid alphaherpesvirus 1, Spheniscid alphaherpesvirus 1.

In some embodiments, the compositions and methods described herein are used to prevent or treat infectious laryngotracheitis. Gallid alphaherpesvirus 1 is known to cause infectious laryngotracheitis (ILT) in chickens and pheasants. ILT is an acute, highly contagious infection characterized by severe dyspnea, coughing, and rales, as well as nasal and ocular discharge, tracheitis, and conjunctivitis. Symptoms manifest about 5-12 days post infection and mortality rates are about 50% in adult birds.

In some embodiments, the compositions and methods described herein are used to prevent or treat amazon tracheitis. Gallid alphaherpesvirus 1 is also known to cause amazon tracheitis, which is a similar disease to ILT, but prevalent amongst companion birds, such as parrots and causes severe diphtheritic lesions in the trachea and bronchi and tracheal necrosis. There currently are no treatment or prevention methods.

In some embodiments, the compositions and methods described herein are used to prevent or treat Pacheco's disease. Psittacid alphaherpesvirus 1 is known to cause Pacheco's disease in parrots. Pacheco's disease is a highly infectious and deadly illness, in which a parrot may or may not develop symptoms, and usually dies within a few days. Symptoms include green colored feces, listlessness, diarrhea, nasal discharge, lack of appetite, swelling, and redness of the eye, tremors, and ruffled feathers. Symptoms usually manifest 3-7 days post infection, but many birds will not display symptoms at all. Current treatment is harsh and is known to cause kidney damage and current prevention includes a two-dose vaccination, wherein the side effects are great and is only recommended for at-risk animals.

In some embodiments, the compositions and methods described herein are used to prevent or treat duck viral enteritis. Anatid alphaherpesvirus 1 is known to cause duck viral enteritis (DVE), which is an acute, highly contagious disease of waterfowls, including ducks, swans, and geese. DVE is characterized by sudden death, high mortality, hemorrhages, and necrosis in internal organs. Considerable losses have been reported in the United States as a result of mass die-offs on duck farms. Symptoms manifest between 3-7 days post infection and mortality is often the first sign of the disease. Other symptoms may include inability to stand, weakness, lack of appetite, extreme thirst, soiled vents, watery and bloody diarrhea, droopiness, and ataxia. Currently, there is no treatment for DVE.

In some embodiments, the compositions and methods described herein are used to prevent or treat Marek's disease. Gallid alphaherpesvirus 2 is known to cause Marek's disease (MDV) in poultry, including chickens, turkeys, and quail. Marek's disease is characterized by the presence of T cell lymphoma as well as infiltration of nerves and organs by lymphocytes. Marek's disease is one of the most ubiquitous avian infections wherein every flock is presumed to be infected, except for those maintained under strict, pathogen-free, conditions. MDV is highly contagious and readily transmitted between birds and may survive months outside the bird, in poultry house litter or dust. Infected birds continue to be carriers for long periods of time and act as sources of the infectious virus. In addition to lymphoid neoplasms, Marek's disease may also induce transient paralysis, early mortality, cytolytic infection, atherosclerosis, and persistent neurological disease. Currently, there is no effective treatment for Marek's disease. Prevention methods include strict sanitation and in ovo or at hatch vaccination.

In some aspects of the present disclosure, the compositions and methods described herein are used to prevent and treat herpesvirus infections in cattle, such as yak, bison, cows, buffalo, and wildebeest. Non-limiting examples of herpesviruses that may infect cattle include Bovine alphaherpesvirus 1, Bovine alphaherpesvirus 2, Bovine alphaherpesvirus 5, Bubaline alphaherpesvirus 1, Alcelaphine gammaherpesvirus 1, Alcelaphine gammaherpesvirus 2, Bovine gammaherpesvirus 6, and Bovine gammaherpesvirus 4.

In some embodiments, the compositions and methods described herein are used to prevent or treat infectious bovine rhinotracheitis. Bovine alphaherpesvirus 1 is known to cause infectious bovine rhinotracheitis (IBR) in cattle. IBR is characterized by acute inflammation of the upper respiratory tract and can also cause abortion. IBR infection affects animal health and productivity causing significant economic losses. Symptoms include fever, loss of appetite, depression, reddening of the mucous membranes, ulceration/reddening of the upper airway, nasal discharge, conjunctivitis, and abortion. There is currently no treatment for IBR and prevention methods include selective culling and the use of live vaccines.

In some embodiments, the compositions and methods described herein are used to prevent or treat meningoencephalitis and respiratory disease. Bovine alphaherpesvirus 5 is known to cause meningoencephalitis and respiratory disease in cattle and sheep. The disease is most common in calves up to 10 months of age. Symptoms include tachycardia, tachypnea, dyspnea, mucoid nasal discharge, hypersalivation, lethargy, opisthotonus, abnormal behavior, blindness, head pressing, proprioceptive deficits, coma, seizures, and sudden death. Currently there is no treatment or specific vaccine.

In some embodiments, the compositions and methods described herein are used to prevent or treat malignant catarrhal fever (MCF). Alcelaphine gammaherpesvirus 1 and 2 and Hippotragine gammaherpesvirus 1 is known to cause malignant catarrhal fever in even-toed ungulates, such as cattle, bison, water buffalo, deer, and pigs. MCF is a very serious, deadly infection wherein symptoms begin to manifest 2 weeks-9 months after exposure. Symptoms include depression, weakness, and diarrhea, but usually death is the first sign of infection. There is currently no treatment for MCF and preventative measures include isolation of infected animals and diagnostic testing before introducing animals in controlled environments, such as a zoo. MCF is an unpredictable disease, wherein some carriers never develop any symptoms. Most new infections occur in animals less than a year old and the mortality rate can be up to 90%.

In some aspects of the present disclosure, the compositions and methods described herein are used to prevent and treat herpesvirus infections in non-human primates such as, macaques, apes, monkeys, tamarins, baboons, marmosets, chimpanzees, lemurs, galagos, ursinus, grivets, gorillas, capuchins, geladas, treeshrews, vervets, gibbons, bonobos, orangutans, tarsiers, lorises, aye-ayes, titis, sakis, and uakaris. Non-limiting examples of herpesviruses that may affect non-human primates include Ateline alphaherpesvirus 1, Cercopithecine alphaherpesvirus 2, Macacine alphaherpesvirus 1, Macacine alphaherpesvirus 2, Macacine alphaherpesvirus 3, Panine alphaherpesvirus 3, Papiine alphaherpesvirus 2, Saimiriine alphaherpesvirus 1, Cercopithecine alphaherpesvirus 9, Aotine betaherpesvirus 1, Cebine betaherpesvirus 1, Cercopithecine betaherpesvirus 5, Macacine betaherpesvirus 3, Macacine betaherpesvirus 8, Mandrilline betaherpesvirus 1, Panine betaherpesvirus 2, Papiine betaherpesvirus 3, Papiine betaherpesvirus 4, Saimiriine betaherpesvirus 4, Macacine betaherpesvirus 9, Callitriine gammaherpesvirus 3, Cercopithecine gammaherpesvirus 14, Gorilline gammaherpesvirus 1, Macacine gammaherpesvirus 4, Macacine gammaherpesvirus 10, Panine gammaherpesvirus 1, Papiine gammaherpesvirus 1, Pongrine gammaherpesvirus 2, Ateline gammaherpesvirus 2, Ateline gammaherpesvirus 3, Macacine gammaherpesvirus 5, Macacine gammaherpesvirus 8, Macacine gammaherpesvirus 11, Macacine gammaherpesvirus 12, Saimiriine gammaherpesvirus 2, and Saguinine gammaherpesvirus 1.

In some embodiments, the compositions and methods described herein are used to prevent or treat cytomegalovirus (CMV). Aotine betaherpesvirus 1, Cebine betaherpesvirus 1, Cercopithecine betaherpesvirus 5, Macacine betaherpesvirus 3, Macacine betaherpesvirus 8, Mandrilline betaherpesvirus 1, Panine betaherpesvirus 2, Papiine betaherpesvirus 3, Papiine betaherpesvirus 4, and Saimiriine betaherpesvirus 4 are known to cause CMV in non-human primates. CMV is a severe and deadly disease that is characterized by fatal encephalitis and encephalomyelitis, lingual ulcers, stomatitis, conjunctivitis, and meningitis. In many cases, initial infection is latent in reservoir hosts, but severe and often fatal when transmitted to other hosts. In particular, Cercopithecine betaherpesvirus 1, typically results in mild symptoms such as, conjunctivitis or oral vesicles in Macaques, but often causes fatal encephalitis and encephalomyelitis when transmitted to humans via a bite or scratch. Currently, there is no treatment and vaccines are inaccessible in many developing countries wherein many of these species live.

In some embodiments, the compositions and methods described herein are used to prevent or treat lymphosarcoma. Callitrichine gammaherpesvirus 3, Cercopithecine gammaherpesvirus 14, Gorilline gammaherpesvirus 1, Macacine gammaherpesvirus 4, Macacine gammaherpesvirus 10, Panine gammaherpesvirus 1, Papiine gammaherpesvirus 1, and Pongine gammaherpesvirus 2 are known to cause lymphosarcoma in non-human primates. These virus are all closely related to human herpesvirus 4, the Epstein-Barr virus, which infects B lymphocytes. Similarly in non-human primates, infection may cause a similar fatal lymphoproliferative disease that resembles human Burkett's lymphoma.

In some aspects of the present disclosure, the compositions and methods described herein are used to prevent and treat herpesvirus infections in reptiles including snakes, lizards, turtles, tortoises, alligators, and crocodiles. Non-limiting examples of herpesviruses that may affect reptiles include Chelonid alphaherpesvirus 5, Testudinid alphaherpesvirus 3, Chelonid alphaherpesvirus 6, Iguanid herpesvirus 2, Emydoidea herpesvirus 1, Pelomedusid herpesvirus 1, Glyptemys herpesvirus 1, Glyptemys herpesvirus 2, Emydid herpesvirus 1, Emydid herpesvirus 2, Terrapene herpesvirus 1, and Terrapene herpesvirus 2.

In some embodiments, the compositions and methods described herein are used to prevent or treat lung-eye-trachea disease (LETV). Chelonid herpesvirus 6 is known to cause lung-eye-trachea disease in turtles. Symptoms include conjunctivitis, stomatitis, pharyngitis, glottitis, tracheitis, and pneumonia and typically manifest about 2-3 weeks post infection. Currently there are no treatments available and prevention is limited to sterilization techniques.

In some embodiments, the compositions and methods described herein are used to prevent or treat fibropapillomatosis. Chenolid alphaherpesvirus 5 is known to cause fibropapillomatosis in sea turtles. Fibropapillomatosis is characterized by the appearance of external tumors that may grow so large as to affect swimming, vision, feeding, and potential escape from predators. The external tumors are caused by excess fibrous connective tissue in both epidermal and dermal skin layers. In addition, about 25% of all turtles with external tumors also have internal tumors primarily in the heart, lung, and kidneys. The only treatment available is surgical removal of the tumors and preventative care is non-existent.

In some aspects of the present disclosure, the compositions and methods described herein are used to prevent and treat herpesvirus infections in felines, including domesticated cats, house-cats, feral cats, and big cats including lions, tigers, jaguars, leopards, pumas, lynx, cheetahs, and cougars. A non-limiting example of a herpesvirus that infect felines is Felid alphaherpesvirus 1.

In some embodiments, the compositions and methods described herein are used to prevent or treat feline viral rhinotracheitis. Felid alphaherpesvirus 1 is known to cause feline viral rhinotracheitis in wild and domestic cats. Feline viral rhinotracheitis is an upper respiratory infection that is characterized by fever, frequent sneezing, inflamed eyes (conjunctivitis), rhinitis, and increased salivation. Symptoms may persist for −10 days, but can last up to 6 weeks. The only treatments available are supportive and preventative care is available in the form of intranasal or intramuscular vaccine.

In some aspects of the present disclosure, the compositions and methods described herein are used to prevent and treat herpesvirus infections in suids, such as pigs, hogs, swine, and boars. Non-limiting examples of herpesviruses that infect suids include Suid alphaherpesvirus 1, Suid betaherpesvirus 2, Suid gammaherpesvirus 3, Suid gammaherpesvirus 4, and Suid gammaherpesvirus 5.

In some embodiments, the compositions and methods described herein are used to prevent or treat pseudorabies. Suid alphaherpesvirus 1 is known to cause pseudorabies in pigs, boar, swine, and hogs, but also can affect cattle, dogs, sheep, and goats. Pseudorabies is a highly contagious infection that causes reproductive issues, such as stillbirth and abortion, as well as respiratory problems and death. Symptoms include incoordination, sneezing, coughing, high mortality, fever, pneumonia, abortions, still births, and small litters. Currently there is no treatment available for pseudorabies and preventative care is limited to biosecurity and vaccination.

In some aspects of the present disclosure, the compositions and methods described herein are used to prevent and treat herpesvirus infections in elephants. Non-limiting examples of herpesviruses that infect elephants include Elephantid betaherpesvirus 1, Elephantid betaherpesvirus 4, and Elephantid betaherpesvirus 5.

In some embodiments, the compositions and methods described herein are used to prevent or treat acute hemorrhagic disease. Elephantid herpesvirus 4 is known to cause acute hemorrhagic disease. Acute hemorrhagic disease is a highly fatal disease the primarily effects young elephants. The mortality rate is around 85% and most clinical signs are nonspecific and may include lethargy, decreased appetite, and tachycardia. There are currently no treatments and preventative care is limited to diagnostic testing before introduction in controlled environments, such as zoos.

In one aspect, provided are compositions comprising one or more antiviral agents useful for the prevention of herpesvirus infection in a herpesvirus seronegative subject, wherein the compositions protect the seronegative subject against the transmission of shedding virus from the seropositive subject. These antiviral compositions for protecting the herpesvirus seronegative subject are optionally also useful in suppressive treatment of herpesvirus seropositive subject. In some aspects of the methods described herein, the herpesvirus compositions further comprise and/or are administered with, one or more additional antiviral agents. In additional embodiments, a composition comprising one or more herpesvirus antiviral agents. As such, the present disclosure, in various embodiments, provides compositions and methods for the prevention of herpesvirus infection in a herpesvirus seronegative subject.

In one aspect of the disclosure, described herein are compositions and methods for the prevention of herpesvirus infection in a herpesvirus seronegative subject at risk for exposure to herpesvirus. In some cases, these compositions are referred to as pre-exposure compositions or herpesvirus pre-exposure compositions. In some embodiments, the herpesvirus seronegative subject is at risk for exposure to herpesvirus through one or more incidents of contact with one or more subjects, where the one or more subjects is herpesvirus seropositive or has an unknown herpesvirus sero-status. In various embodiments, the pre-exposure compositions described herein comprise one or more antiviral agents at a low dose, for example, a dose that is the same or 10%, 20%, 30%, 40%, 50%, 60%, 70%, 80%, or 90% smaller than the dose typically administered to a herpesvirus seropositive subject for suppression therapy.

In another aspect, described herein are compositions and methods for the prevention of herpesvirus infection in a herpesvirus seronegative subject after exposure to herpesvirus. In some cases, these compositions are referred to as post-exposure compositions or herpesvirus post-exposure compositions. In some embodiments, the herpesvirus seronegative subject is exposed to herpesvirus through one or more incidents of contact with one or more subjects, where the one or more subjects is herpesvirus seropositive. In various embodiments, the post-exposure compositions described herein comprise one or more antiviral agents at a high dose, for example, a dose that is the same or 10%, 20%, 30%, 40%, 50%, 60%, 70%, 80%, or 90% greater than the dose typically administered to a herpesvirus seropositive subject for therapy after an initial outbreak.

In some embodiments, a herpesvirus composition or an antiviral composition refers to a pre-exposure composition. In some embodiments, a herpesvirus composition or an antiviral composition refers to a post-exposure composition. In some embodiments, a herpesvirus composition or an antiviral composition refers to a composition comprising one or more antivirals for suppressive therapy of a herpesvirus seropositive subject. In some embodiments, a herpesvirus composition or an antiviral composition refers to both pre-exposure and post-exposure compositions. In further embodiments, a herpesvirus composition or an antiviral composition refers to pre-exposure compositions, post-exposure compositions, and compositions for suppressive therapy.

In some embodiments, the methods described herein comprise administering an antiviral agent to a subject of unknown sero-status in an amount that is both effective for prevention of herpesvirus infection in a herpesvirus seronegative subject and effective for herpesvirus suppression therapy in a herpesvirus seropositive subject. In some cases, the effective amount is a low dose of an antiviral agent. In such instances, herpesvirus testing is not required for administering an antiviral composition. Common methods for testing for herpesvirus sero-status include immunoassays, cell culturing, and nucleic acid detection, for example, using polymerase chain reaction. As the standard of care does not currently provide test kits readily performed without assistance of a laboratory and health care personal, herpesvirus sero-status is not easily ascertained. In various aspects, the methods and compositions provided eliminate the need for determining sero-status.

In some embodiments, provided herein are methods for administering an antiviral composition to a subject of known herpesvirus seropositive status. In some cases, the herpesvirus seropositive subject is administered a same dosage of an antiviral that a herpesvirus seronegative subject would be administered to prevent herpesvirus infection.

The methods for preventing herpesvirus infection in a herpesvirus seronegative subject, in various embodiments, comprise administering to the herpesvirus seronegative subject a composition comprising one or more antiviral agents. In some embodiments, the methods comprise giving it up to 3 days prior to when semen is taken from a male, and administration can occur up to 3 days prior to when a female is inseminated. In some embodiments, the methods comprise determining a mechanism of administering the herpesvirus composition. In some embodiments, the methods comprise determining an antiviral agent dosage amount effective to suppress herpesvirus replication, herpesvirus re-activation, or both herpesvirus replication and herpesvirus re-activation. In some embodiments, the methods comprise determining a route of administration of the composition and a delivery mechanism, for example, a tablet, a capsule, a soft chew, a gel, a paste, a powder, a bait drop, or a liquid.

The terms “synergy” and “synergistic” mean that the effect achieved with the compounds used together is greater than additive sum of effects that are achieved when using the compounds separately, i.e. greater than what would be predicted based on the two active ingredients administered separately. A synergistic effect may be attained when the compounds are: 1) co-formulated such that they are administered or delivered simultaneously in a combined formulation; 2) delivered or administered in separate formulations either in alternation or in parallel; or 3) by some other regimen. When delivered/administered in alternation, the synergistic effect may be obtained when they are done in a sequential manner e.g. in separate tablets, pills, capsules or injections by different syringes. In general, during alternation therapy, an effective dose of each active ingredient is administered sequentially whereas in combination therapy, effective doses are administered together. A synergistic antiviral effect is an antiviral effect where the combined effect is greater than the predicted additive effects of each individual component.

The term “physiologically functional derivative” means a pharmaceutically active compound with equivalent on near equivalent physiological functionality to valacyclovir or famciclovir when administered in combination with another pharmaceutically active compound in a combination of the invention. As used herein, “physiologically functional derivative” includes a physiologically acceptable salt, ester, proagent, solvate, stereoisomer including enantiomer, distereomer, or stereoisomerically enriched or racemic mixture, or any other compound which upon administration to a subject in need, is capable of providing directly or indirectly such a compound or an antivirally active metabolite or reside thereof.

It has recently been shown that treatment with valacyclovir before exposure could prevent herpes infection including latency. Thus, treatment with valacyclovir, famciclovir alone or in combination will prevent herpes-related illness.

The present invention provides novel combinations of two or more active ingredients being employed together. In some embodiments, a synergistic antiviral effect is achieved. The term “synergistic antiviral effect” is used to denote an antiviral effect that is greater than the predicted purely additive effects of using each antiviral component individually.

While it is possible for the active ingredients of the combination to be administered alone and separately, it is preferable to administer them as a combination. A two-part or three-part combination may be administered simultaneously or sequentially. When administered sequentially, the combination may be administered in one, two, or three administrations. Examples include parenteral administration, such as an intravenous injection or infusion, intramuscular injection, or subcutaneaous injection, or oral administration, such as a single soft chew, tablet, capsule, paste, powder, a bait drop, or liquid including both valacyclovir and famciclovir, parenteral administration, such as an intravenous injection or infusion, intramuscular injection, or subcutaneaous injection, or oral administration, such as a single soft chew, tablet, capsule, paste, powder, a bait drop, or liquid of valacyclovir and a single soft chew, tablet, capsule, paste, powder, a bait drop, or liquid of famciclovir, two soft chews tablet, capsule, paste, powder, a bait drop, or liquid of valacyclovir and a single soft chew, tablet, capsule, paste, powder, a bait drop, or liquid of famciclovir, and parental administration, such as two intravenous injections or infusions, intramuscular injections, or subcutaneous injections, or oral administration, such as two soft chews, tablets, capsules, paste, powders, bait drops, or liquids of valacyclovir and parental administration, such as two intravenous injections or infusions, intramuscular injections, or subcutaneous injections, or oral administration, such as two soft chews, tablets, capsules, pastes, powders, bait drops, or liquids of famciclovir.

The compounds of the combination may be administered 1) simultaneously by combination of the active ingredients in a co-formulation or 2) by alternation, i.e. delivering the active ingredients sequentially, serially, in parallel or simultaneously in spate pharmaceutical formulations. In alternation therapy, the delay in administering the second (or optionally the third compound) would be such as to note lose the synergistic effect of the combination. The combination can be administered in either co-formulation or alternation therapy to achieve peak plasma concentrations such as but not limited to 0.001 to 200 μM.

When the individual active ingredients of the combination are administered separately, they are generally each presented as a pharmaceutical formulation. It will be understood that the administration of the combination of the invention by means of a single pack, or packs of each formulation, within a package insert instructing the handler as to the correct use of the invention is a desirable additional feature of this invention.

The combination therapies of the invention include 1) a combination of valacyclovir and famciclovir or 2) a combination containing a physiologically functional derivative of either or both thereof.

The combination may be formulated in a unit dosage formulation comprising a fixed amount for a periodic, e.g. daily dose or subdose of the active ingredients.

Pharmaceutical formulations according to the present invention comprise a combination according to the invention together with one or more pharmaceutically acceptable carriers or excipients and optionally other therapeutic agents. Pharmaceutical formulations containing the active ingredient may be in any form suitable or the intended method of administration.

The combinations of the invention may conveniently be presented as a pharmaceutical formulation in a unitary dosage form. A convenient unitary dosage formulation contains the active ingredient in any amount from 0.01 mg/kg to 500 mg/kg each, for example, but not limited to 1 mg/kg to 250 mg/kg. The synergistic effects of valacyclovir in combination with famciclovir may be realized over a wide ratio for example (1:100 to 100:1 (valacyclovir:famciclovir). In certain embodiments, the ratio can range from 1:5 to 5:1, for example, about 1:5, about 1:4, about 1:3, about 1:2, about 1:1, about 2:1, about 3:1, about 4:1, or about 5:1. In certain embodiments, the range is from about 1:2 to about 2:1. In another embodiment, there will be an approximately equal amount of valacyclovir and famciclovir. In other exemplary co-formulations, there may be more or less valacyclovir than famciclovir. Other ratios and amounts of the compounds are contemplated within the scope of the invention.

It will be appreciated by those skilled in the art that the amount of active ingredients in the combination of the invention required for use in treatment will vary according to a variety of factors including the nature of the condition being treated, the age and condition of the subject and will ultimately be at the discretion of the health care provider or treating veterinarian. Factors include by are not limited to age, weight, general condition, nature and severity of the disease to be treated.

It is also possible to combine any two of the active ingredients in a unitary dosage form for simultaneous or sequential administration with a third active ingredient. The three-part combination may be administered simultaneously or sequentially. When administered sequentially, the combination may be administered in one, two or three administrations. Third active ingredients have anti-herpesvirus activity. Exemplary third active ingredients to be administered in combination with valacyclovir and famciclovir include but are not limited to particularly useful third agents, by way of example and not limitation are helicase primase inhibitors, SM inhibitors, spironolactone, emtricitabine and lamivudine, PEGylated interferon, ribavirin, boceprevir, telaprevir, simeprevir, sofosbuvir, ledipasvir/sofobuvir, tenofovir, ombitasvir, paritaprevir, ritonavir.

Formulation of the present invention suitable for oral administration may be as capsule, tablet, soft chews, gels, liquids, powders, bait drops, or pastes each containing a predetermined amount of the active ingredients as a powder or granules; as a solution or a suspension in aqueous or non-aqueous liquid; or as an oil-in-water emulsion or a water-in-oil liquid emulsion. Formulation of the present invention suitable for oral administration may be as a powder or aqueous or non-aqueous liquid, or oil-in-water emulsion or water-in-oil emulsion administered as a predetermined amount of active ingredient into daily feed or water supply.

Formulation of the present invention suitable for parenteral administration may be as intravenous injection or infusion, intramuscular injection, or subcutaneous injection each containing a predetermined amount of the active ingredients as a solution or suspension in aqueous liquid.

In some embodiments, the pharmaceutical composition of the present invention may be formulated for long-acting administration. The term “long acting” as used herein refers to a pharmaceutical formulation which provides prolonged, sustained or extended release of the pharmaceutical composition or its pharmaceutically acceptable carrier to the general systemic circulation of a subject or to local sites of action in a subject. This term may further refer to a pharmaceutical formulation which provides prolonged, sustained, controlled or extended duration of action (pharmacokinetics) of the active substance in a subject.

Provided herein, in various aspects, are methods for the prevention of viral infection in a subject comprising administering to the subject a composition comprising an effective dose of at least one antiviral agent. The methods and compositions described herein relate to any infective virus. In some embodiments, reference to a virus is not limited to a single virus and is inclusive of one or more infective viruses. In exemplary embodiments, a virus is at least one Equine Herpesvirus (EHV), at least one Gallid herpesvirus (GHV), and/or at least one Bovine herpesvirus (BHV). In various instances, reference to EHV is inclusive of all infectious viruses and is not limited to herpesvirus. In non-limiting examples, at least one virus includes at least one virus belonging to the family of herpesvirus, hepatitis virus, pox virus, calicivirus, adenovirus, parvovirus, coronavirus, immunodeficiency virus, mosaic virus, paramyxovirus, retrovirus, papillomavirus, rhinovirus, norovirus, poliovirus, rotavirus, rubivirus, zika virus, influenza, leukemia virus, picornavirus, syncytial virus, enterovirus, rubella virus, neurotropic virus, or oncovirus. In some embodiments, the picornavirus family includes but is not limited to picornavirus, poliovirus, rhinovirus, enterovirus (coxsackie virus), hepatitis (hepatitis virus type A, hepatitis virus type B, hepatitis virus type C), aphthovirus, parechovirus, and encephalomyocarditis virus.

In some embodiments, the herpesvirus includes, but is not limited to, Gallid alphaherpesvirus 1 (GaAHV1), Psittacid alphaherpesvirus 1 (PsAHV1), Anatid alphaherpesvirus 1 (AnAHV1), Columbid alphaherpesvirus 1 (CoAHV1), Gallid alphaherpesvirus 2 (GaAHV2), Gallid alphaherpesvirus 3 (GaAHV3), Meleagrid alphaherpesvirus 1 (MeAHV1), Spheniscid alphaherpesvirus 1 (SpAHV1), Chelonid alphaherpesvirus 5 (ChAHV5), Testudinid alphaherpesvirus 3 (TeAHV3), Ateline alphaherpesvirus 1 (AtAHV1), Bovine alphaherpesvirus 2 (BoAHV2), Cercopithecine alphaherpesvirus 2 (CeAHV2), Human alphaherpesvirus 1 (HuAHV1), Human alphaherpesvirus 2 (HuAHV2), Leporid alphaherpesvirus 4 (LeAHV4), Macacine alphaherpesvirus 1 (McAHV1), Macacine alphaherpesvirus 2 (McAHV2), Macropodid alphaherpesvirus 1 (MaAHV1), Macropodid alphaherpesvirus 2 (MaAHV2), Panine alphaherpesvirus 3 (PnAHV3), Papiine alphaherpesvirus 2 (PaAHV2), Pteropodid alphaherpesvirus 1 (PtHAV1), Saimiriine alphaherpesvirus 1 (SaAHV1), Bovine alphaherpesvirus 1 (BoAHV1), Bovine alphaherpesvirus 5 (BoAHV5), Bubaline alphaherpesvirus 1 (BuAHV1), Canid alphaherpesvirus 1 (CaAHV1), Caprine alphaherpesvirus 1 (CpAHV1), Cercopithecine alphaherpesvirus 9 (CeAHV9), Cervid alphaherpesvirus 1 (CvAHV1), Cervid alphaherpesvirus 2 (CvAHV2), Cerbid alphaherpesvirus 3 (CvAHV3), Equid alphaherpesvirus 8 (EqAHV8, EHV-8), Equid alphaherpesvirus 9 (EqAHV9, EHV-9), Felid alphaherpesvirus 1 (FeAHV1), Human alphaherpesvirus 3 (HuAHV3), Monodontid alphaherpesvirus 1 (MoAHV1), Phocid alphaherpesvirus 1 (PcAHV1), Suid alphaherpesvirus 1 (SuAHV1), Chelonid alphaherpesvirus 6 (ChAHV6), Aotine betaherpesvirus 1 (AoBHV1), Cebine betaherpesvirus 1 (CbBHV1), Cercopithecine betaherpesvirus 5 (CeBHV5), Human betaherpesvirus 5 (HuBHV5), Macacine betaherpesvirus 3 (McBHV3), Macacine betaherpesvirus 8 (McBHV8), Mandrille betaherpesvirus 1 (MdBHV1), Panine betaherpesvirus 2 (PnBHV2), Papiine betaherpesvirus 4 (PaBHV4), Saimiriine betaherpesvirus 4 (SaBHV4), Murid betaherpesvirus 1 (MuBHV1), Murid betaherpesvirus 2 (MuBHV2), Murid betaherpesvirus 8 (MuBHV8), Elephantid betaherpesvirus 1 (E1BHV1), Elephantid betaherpesvirus 4 (E1BHV4), Elephantid betaherpesvirus 5 (E1BHV5), Caviid betaherpesvirus 2 (CdBHV2), Miniopterid betaherpesvirus 1 (MnBHV1), Tupaiid betaherpesvirus 1 (TuBHV1), Human betaherpesvirus 7 (HuHBV7), Human betaherpesvirus 6A (HuBHV6A), Human betaherpesvirus 6B (HuBHV6B), Macacine betaherpesvirus 9 (McBHV9), Murid betaherpesvirus 3 (MuHBV3), Suid betaherpesvirus 2 (SuBHV2), Dephinid gammaherpesvirus 1 (DeGHV1), Callitrichine gammaherpesvirus 3 (C1GHV3), Cercopithecine gammaherpesvirus 14 (CeGHV14), Gorilline gammaherpesvirus 1 (GoGHV1), Human gammaherpesvirus 4 (HuGHV4), Macacine gammaherpesvirus 4 (McGHV4), Macacine gammaherpesvirus 10 (McGHV10), Panine gammherpesvirus 1 (PnGHV1), Papiine gammaherpesvirus 1 (PaGHV1), Pongine gammaherpesvirus 2 (PoGHV2), Alcelaphine gammaherpesvirus 1 (AlGHV1), Alcelaphine gammaherpesvirus 2 (AlGHV2), Bovine gammaherpesvirus 6 (BoGHV6), Caprine gammaherpesvirus 2 (CpGHV2), Hippotragine gammaherpesvirus 1 (HiGHV1), Ovine gammaherpesvirus 2 (OvGHV2), Suid gammaherpesvirus 3 (SuGHV3), Suid gammaherpesvirus 4 (SuGHV4), Suid gammaherpesvirus 5 (SuGHV5), Phascolarctid gammaherpesvirus 1 (PcGHV1), Vombatid gammaherpesvirus 1 (VoGHV1), Vespertilionid gammaherpesvirus 3 (VeGHV3), Felid gammaherpesvirus 1 (FeGHV1), Mustelid gammaherpesvirus 1 (MusGHV1), Phocid gammaherpesvirus 3 (PhGHV3), Verspertilionid gammaherpesvirus 1 (VeGHV1), Ateline gammaherpesvirus 2 (AtGHV2), Ateline gammaherpesvirus 3 (AtGHV3), Bovine gammaherpesvirus 4 (BoGHV4), Cricetid gammaherpesvirus 2 (CrGHV2), Human gammaherpesvirus 8 (HuGHV8), Macacine gammaherpesvirus 5 (McGHV5), Macacine gammaherpesvirus 8 (McGHV8), Macacine gammaherpesvirus 11 (McGHV11), Macacine gammaherpesvirus 12 (McGHV12), Murid gammaherpesvirus 4 (MuGHV4), Murid gammaherpesvirus 7 (MuGHV7), Saimiriine gammaherpesvirus 2 (SaGHV2), Equid gammaherpesvirus 7 (EGHV7, EHV-7), Phocid gammaherpesvirus 2 (PhoGHV2), Saguinine gammaherpesvirus 1 (SgGHV1), Iguanid herpesvirus 2 (IgHV2), Anguillid herpesvirus 1 (AngHV1), Cyprinid herpesvirus 1 (CyHV1), Cyprinid herpesvirus 2 (CyHV2), Cyprinid herpesvirus 3 (CyHV3), Ictalurid herpesvirus 1 (IcHV1), Ictalurid herpesvirus 2 (IcHV2), Acipenserid herpesvirus 1 (AciHV1), Acipenserid herpesvirus 2 (AciHV2), Salmonid herpesvirus 1 (SalHV1), Salmonid herpesvirus 2 (SalHV2), Salmonid herpesvirus 3 (SalHV3), Gadid herpesvirus 1 (GaHV1), Ranid herpesvirus 1 (RaHV1), Ranid herpesvirus 2 (RaHV2), Pilchard herpesvirus, Tilapia herpesvirus, Percid herpesvirus 1 (PeHV1), Emydoidea herpesvirus 1 (EBHV-1), Pelomedusid herpesvirus 1, Glyptemys herpesvirus 1 (GlyHV-1), Glyptemys herpesvirus 2 (GlyHV-2), Emydid herpesvirus 1 (EmyHV-1), Emydid herpesvirus 2 (EmyHV-2), Terrapene herpesvirus 1 (TerHV-1), Terrapene herpesvirus 2 (TerHV-2), Equine Herpesvirus-1 (EHV-1), Equine Herpesvirus-2 (EHV-2), Equine Herpesvirus-3 (EHV-3), Equine Herpesvirus-4 (EHV-4), and Equine Herpesvirus-5 (EHV-5). EHV-1 typically causes neurological disease, respiratory disease, abortion, and neonatal death. EHV-3 typically causes venereal disease, and EHV-4 typically causes nonfatal respiratory tract disease.

In some embodiments, a viral infection refers to a verified presence of a viral antibody, viral antigen, and/or viral nucleic acid in a subject using viral diagnostic tests known to those skilled in the art (e.g. immunoassays such as ELISAs, Western blot, and lateral flow assays; and PCR). In exemplary embodiments, a herpesvirus infection refers to the verified presence of anti-herpesvirus antibody or herpesvirus nucleic acid. In some instances, a herpesvirus seropositive subject is a subject who has detectable anti-herpesvirus antibody in their blood. In some instances, a herpesvirus seronegative subject is a subject who does not have detectable anti-herpesvirus antibody in their blood. In some cases, a herpesvirus seronegative subject has been exposed to herpesvirus, but herpesvirus has not established an infection and/or the subject has not yet seroconverted so that anti-herpesvirus antibodies are not yet detectable.

Provided herein, in various aspects, are compositions and methods for the prevention of herpesvirus infection in a herpesvirus seronegative subject from a herpesvirus seropositive subject, the methods comprising administering to the herpesvirus seronegative subject a composition comprising an effective dose of a first antiviral agent. In various embodiments, the composition prevents the establishment of a new persistent infection in the seronegative subject after exposure to herpesvirus. In some embodiments, the composition is administered prior to herpesvirus exposure from a seropositive subject, e.g., the composition is a pre-exposure composition. In some embodiments, the composition is administered after herpesvirus exposure from a seropositive subject, e.g., the composition is a post-exposure composition. In some embodiments, the composition is administered prior to and after herpesvirus exposure to the seronegative subject.

In additional aspects, provided herein are methods for the prevention of herpesvirus infection in a herpesvirus seronegative subject from a herpesvirus seropositive subject, the methods comprising administering to the herpesvirus seronegative subject a composition comprising a first effective dose of a first antiviral agent and further comprising administering to the herpesvirus seropositive subject a second composition comprising a second effective dose of a second antiviral agent. In some embodiments, the first antiviral agent and the second antiviral agent comprise the same active agent. In some embodiments, the first antiviral agent and the second antiviral agent comprise different active agents. In some embodiments, the composition comprising the first antiviral agent further comprises or is administered with an additional antiviral agent, wherein the additional antiviral agent is useful for the prevention of herpesvirus.

In some embodiments, an antiviral agent in a composition for administration to a herpesvirus seronegative subject inactivates herpesvirus shed by a seropositive subject and transferred to the herpesvirus seronegative subject. In some cases, the antiviral agent administered to a herpesvirus seronegative subject that has been exposed to herpesvirus reduces the amount of viable virus so that the virus is not able to produce a sustainable infection in the subject.

In some embodiments, the compositions useful for the methods of reducing herpesvirus viral load levels in seronegative subjects comprise two or more antivirals. In some cases, at least two of the two or more antivirals employ different mechanisms for inhibiting viral infection. In some cases, at least two of the two or more antivirals competitively inhibit excretion allowing for a synergistic and sustainable plateaus in plasma AUC. In some cases, an antiviral administered to a seronegative subject is the same as an antiviral administered to a seropositive subject. In some cases, an antiviral administered to a seronegative subject is different than an antiviral administered to a seropositive subject. In some cases, two or more antivirals are administered within the same composition, or two or more antivirals are administered within two or more different compositions, to create a cumulative effect incorporating the efficacy of the various agents against the target virus. For example, a combination of antivirals (within the same composition or co-administered within different compositions) reduces the rate of viral load levels to a greater degree than employing one of the antivirals in the combination. As another example, a combination of antivirals reduces the rate of viral shedding and transmission rate to a greater degree than employing only one of the antivirals within the combination. In some cases, combination therapy is effective in situations where a single agent may be ineffective due to, for example, agent resistance, dosing mistakes, and/or non-compliance. In some cases, combinations of antivirals having different mechanisms of action provide enhanced therapeutic effectiveness over a single antiviral.

A variety of antiviral agents are useful in the methods and compositions described herein for both the prevention of viral transmission from seropositive subjects to seronegative subjects and the prevention of viral infection in seronegative subjects exposed to virus. In some embodiments, a seronegative subject is treated with a composition comprising an antiviral for the prevention of viral infection in the seronegative subject. In exemplary embodiments, antiviral agents administered to a seronegative subject for infection prevention include those useful for acute and/or suppressive therapy in seropositive subject. In some embodiments, antiviral agents are useful in the methods and compositions described herein for the prevention of herpesvirus transmission from herpesvirus seropositive subjects to herpesvirus seronegative subjects. In some embodiments, a herpesvirus seronegative subject is treated with a composition comprising an antiviral for the prevention of herpesvirus infection in the seronegative subject. In exemplary embodiments, antiviral agents administered to a herpesvirus seronegative subject for infection prevention include those useful for acute and/or suppressive therapy in herpesvirus seropositive subjects.

Antiviral agents useful in the compositions and methods described herein include, without limitation, any compound or a pharmaceutically acceptable salt or solvate thereof, which is capable of inhibiting replication of a virus in a cell, such as a cell in a subject, or which is effective in treating, preventing, or delaying the onset or progression of viral infection or viral diseases or conditions arising from viral infection. In some embodiments, an antiviral agent prevents or delays initial infection of a subject exposed to a virus. In some embodiments, an antiviral agent reduces the viral burden in a subject infected with a virus. In some embodiments, an antiviral agent prolongs an asymptomatic phase of a viral infection. In some embodiments, an antiviral agent maintains low viral loads of virus in infected subjects. In some embodiments, an antiviral agent increases overall health or quality of life in a subject infected with a virus. In some embodiments, an antiviral agent prolongs life expectancy of a subject infected with a virus. In a non-limiting example, the effect(s) of an antiviral agent on a subject are determined by comparing a subject treated with an antiviral with a subject not treated with an antiviral, for instance, in a clinical trial to determine whether treatment with an antiviral agent is effective in treating, preventing or delaying the onset or progression of viral infection or viral diseases or conditions arising from viral infection. In some instances, the virus is a herpesvirus.

Suitable antiviral agents useful for the compositions and methods herein include, without limitation, “off-label” agents useful for treating viral diseases or conditions, hepatitis antivirals, pox virus antivirals, calicivirus antivirals, adenovirus antivirals, parvovirus antivirals, coronavirus antivirals, immunodeficiency virus antivirals, mosaic virus antivirals, paramyxovirus antivirals, retrovirus antivirals, papillomavirus antivirals, herpesvirus antivirals, rhinovirus antivirals, norovirus antivirals, poliovirus antivirals, rotavirus antivirals, rubivirus antivirals, zika virus antivirals, leukemia virus antivirals, picornavirus antivirals, syncytial virus antivirals, enterovirus antivirals, rubella virus antivirals, neurotropic virus antivirals, oncovirus antivirals, and influenza antivirals. “Off-label” agents include agents such as cancer agents which are useful for the treatment of a viral infection or diseases or conditions resulting therefrom, which are not currently approved for viral treatment. Antiviral agents include viral protease inhibitors, viral reverse transcriptase inhibitors, viral entry inhibitors viral co-receptor inhibitors, viral fusion inhibitors, viral maturation inhibitors, viral integrase inhibitors and viral immunogens. In some embodiments, an antiviral agent comprises RNA, e.g., siRNA, which target viral nucleic acids. In some embodiments, an antiviral agent comprises a microbicide.

In some embodiments, an antiviral agent useful for the prevention of a viral disease comprises an antiviral agent that when administered to a subject seronegative for infection with the viral disease, the seronegative subject has a decreased chance of becoming infected and seropositive for the virus. For example, an effective amount of an antiviral agent administered alone or in combination with one or more additional antiviral agents to a seronegative subject or population of subjects, reduces the risk of the subject or population of subjects becoming infected with a virus by at least about 10%, 20%, 30%, 40%, 50%, 60%, 70%, 80%, 90%, 95%, or 100%.

In some embodiments, an antiviral agent useful for the prevention of a viral disease is a herpesvirus antiviral. In some cases, the viral disease is caused by a herpesvirus, for example, EHV. In some cases, the antiviral agent is useful for the prevention of two or more viral diseases. In some embodiments, a herpesvirus antiviral includes an antiviral antibody, such as an anti-herpesvirus antibody. Antibodies include monoclonal and polyclonal antibodies and include fragments and portions thereof, for example, Fab fragments.

Herpesvirus antiviral agents useful in the compositions and methods described herein include nucleoside analogs that selectively target viral DNA polymerase. In some cases, a herpesvirus antiviral agent targets thymidine kinase. For example, herpesvirus antiviral agents can be analogs of the natural nucleoside triphosphate dGTP, and are selectively phosphorylated in virus infected cells by viral thymidine kinase. The phosphorylation of these analogs is minimal in uninfected cells, and cellular DNA polymerases have lower affinities for the antiviral triphosphates compared with HSV polymerases. Viral DNA polymerase is selectively inhibited as viral incorporation of the analogue triphosphate into a growing DNA chain prevents continued chain elongation. Non-limiting examples of these nucleoside triphosphate analogs include acyclovir, valacyclovir, penciclovir, and famciclovir. In some cases, a herpesvirus antiviral agent targets a protease. In some cases, a herpesvirus antiviral agent inhibits viral helicase-primase complex. A non-limiting example of a helicase-primase inhibitor is pritelivir. In some cases wherein a virus is resistant to a nucleoside analog, the virus may be susceptible to a helicase-primase inhibitor or a combination of nucleoside analog and helicase-primase inhibitor. In some instances, a composition described herein comprises both a nucleoside analog and a helicase-primase inhibitor.

Non-limiting examples of herpesvirus antiviral agents for use in the compositions and methods described herein include valacyclovir, acyclovir, famciclovir, pritelivir, penciclovir, ganciclovir, valganciclovir, cidofovir, foscarnet, darunavir, glycyrrhizic acid, glutamine, FV-100, ASP2151, me-609, ASP2151, topical VDO, PEG-formulation (Devirex AG), vidarabine, cidofovir, crofelemer (SP-303T), EPB-348, CMX001, V212, NB-001, squaric acid, ionic zinc, sorivudine (ARYS-01), trifluridine, 882C87, merlin (ethanol and glycolic acid mixture), vitamin C, AIC316, versabase gel with Sarracenia purpurea, UB-621, lysine, edoxudine, brivudine, cytarabine, docosanol, tromantadine, resiquimod (R-848), imiquimod, resiquimod, tenofovir, tenofovir disoproxil fumarate, tenofovir alafenamide fumarate, and salts, solvates, and/or combinations thereof.

In some embodiments, an antiviral agent of a composition described herein comprises a vaccine that provides acquired immunity to a disease caused by a virus. In some cases, the composition further comprises an adjuvant. In some embodiments, described herein are compositions comprising one or more viral vaccines useful for the prevention of herpesvirus transmission In some cases, the viral vaccine compositions further comprise or are administered with one or more additional antiviral agents.

In some embodiments, the dose of the first antiviral agent is from about 0.01 mg/kg to about 500 mg/kg. As non-limiting examples, the composition comprises about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of the first antiviral agent. The dosage requirements of the antiviral agents may vary based on the severity of the symptoms presented and the particular subject being treated. One skilled in the art will appreciate that the optimum dosage for a smaller animal, for example, a rodent or a chicken is very different from the optimum dose for a larger animal, for example, a horse or elephant. The dosage may be increased or decreased until the optimum effect under the circumstances is reached. Precise dosages will be determined by the administering veterinarian based on experience with the individual subject treated.

In some embodiments, the method comprises determining a dose that provides an effective amount of an antiviral agent according to body weight. As used herein, body weight and body mass are used interchangeably and refer to the mass of a subject rather than the force exerted in a gravitational field. The average body weight of a fish ranges from about less than 1 mg to about 19,000 kg. The average body weight of a reptile ranges from about 10 g to 1000 kg. The average body weight of a bird ranges from about 1 g to 160 kg. The average body weight of an amphibian ranges from about 0.01 g to about 50 kg. The average body weight of a mammal belonging to the order Rodentia ranges from about 3 g to about 70 kg. The average body weight of a mammal belonging to the order Chiroptera ranges from about 2 g to about 2 kg. The average body weight of a mammal belonging to the order Soricomorpha ranges from about 2 g to 1 kg. The average body weight of a mammal belonging to the order Primate ranges from about 30 g to about 200 kg. The average body weight of a mammal belonging to the order Carnivora, subclade Feliformia ranges from about 2 kg to about 300 kg. The average body weight of a mammal belonging to the order Carnivora, subclade Caniforma ranges from about 1 kg to about 2500 kg. The average body weight of a mammal belonging to the order Artiodactyla ranges from about 1.5 kg to about 4500 kg. The average body weight of a mammal belonging to the order Diprotodontia ranges from about 10 g to about 95 kg. The average body weight of a mammal belonging to the order Lagomorpha ranges from about 100 g to about 5 kg. The average body weight of a mammal belonging to the order Didelphimorphia ranges from about 1 kg to about 6 kg. The average body weight of a mammal belonging to the order Cetacea ranges from about 25 kg to about 199 metric tons. The average body weight of a mammal belonging to the order Dasyuromorphia ranges from about 15 g to about 2 kg. The average body weight of a mammal belonging to the order Afrosoricida ranges from about 1 g to about 200 g. The average body weight of a mammal belonging to the order Erinaceidae ranges from about 40 g to 1.5 kg. The average body weight of a mammal belonging to the order Cingulata ranges from about 100 g to about 45 kg. The average body weight of a mammal belonging to the order Permelemorphia ranges from about 140 g to about 4 kg. The average body weight of a mammal belonging to the order Scandentia ranges from about 50 g to about 300 g. The average body weight of a mammal belonging to the order Perissodactyla ranges from about 150 kg to about 4500 kg. The average body weight of a mammal belonging to the order Macroscelidea ranges from about 50 g to 700 g. The average body weight of a mammal belonging to the order Pilosa ranges from about 300 g to about 40 kg. The average body weight of a mammal belonging to the order Monotrema ranges from about 0.5 kg to about 10 kg. The average body weight of a mammal belonging to the order Sirenia ranges from about 400 kg to about 2000 kg. The average body weight of a mammal belonging to the order Proboscidea ranges from about 1800 kg to about 6500 kg.

In some embodiments, the method comprises determining a dose that provides an effective amount of a second antiviral agent according to body mass. In some embodiments, the method comprises determining doses of first and second antiviral agents administered in combination according to body mass. In some embodiments, the doses take into account the affinity of an antiviral agent for its target. In some embodiments, such doses take into account the intracellular half-life of an antiviral agent. In some embodiments, the doses take into account the plasma half-life of the antiviral agent. As non-limiting examples, the composition comprises about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg, or from about 0.01 mg/kg to about 500 mg/kg, or about 0.1 mg/kg to about 500 mg/kg, or from about 1 mg/kg to about 500 mg/kg, or from about 0.01 mg/kg to about 250 mg/kg, or from about 0.1 mg/kg to about 250 mg/kg, or from about 1 mg/kg to about 250 mg/kg, of from about 0.01 mg/kg to about 100 mg/kg, or from about 0.1 mg/kg to about 100 mg/kg, or from about 1 mg/kg to about 100 mg/kg, or from greater than 1 mg/kg to about 500 mg/kg, of from greater than 5 mg/kg to about 500 mg/kg body weight of an antiviral agent.

It has been surprisingly found that certain antiviral agents are more effective for treatment of herpesvirus infection or for prophylaxis of herpesvirus in a subject when administered in combination. Non-limiting uses include: pre-exposure prophylaxis (e.g. preventing infection in a seronegative subject); treatment of initial infection (e.g., before, or as a seronegative animal is becoming seropositive); prophylaxis (preventing a recurrent illness) in a seropositive animal and preventing neurologic manifestations of the illness (for example, EHM); and treatment of a recurrent infection in a seropositive animal. Further, the antiviral agents are useful for treatment or prophylactic treatment of a disease or condition associated with herpesvirus infection.

In certain embodiments, the antiviral agents target the same polymerase. In one exemplary embodiment, the first antiviral agent is valacyclovir and the second antiviral agent is famciclovir. In embodiments involving combinations of a first and second antiviral agent, the doses of the agents can be the same or different. As non-limiting examples, the composition comprises about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg, or from about 0.01 mg/kg to about 500 mg/kg, or about 0.1 mg/kg to about 500 mg/kg, or from about 1 mg/kg to about 500 mg/kg, or from about 0.01 mg/kg to about 250 mg/kg, or from about 0.1 mg/kg to about 250 mg/kg, or from about 1 mg/kg to about 250 mg/kg, of from about 0.01 mg/kg to about 100 mg/kg, or from about 0.1 mg/kg to about 100 mg/kg, or from about 1 mg/kg to about 100 mg/kg, or from greater than 1 mg/kg to about 500 mg/kg, of from greater than 5 mg/kg to about 500 mg/kg body weight of a first antiviral agent and about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg, or from about 0.01 mg/kg to about 500 mg/kg, or about 0.1 mg/kg to about 500 mg/kg, or from about 1 mg/kg to about 500 mg/kg, or from about 0.01 mg/kg to about 250 mg/kg, or from about 0.1 mg/kg to about 250 mg/kg, or from about 1 mg/kg to about 250 mg/kg, of from about 0.01 mg/kg to about 100 mg/kg, or from about 0.1 mg/kg to about 100 mg/kg, or from about 1 mg/kg to about 100 mg/kg, or from greater than 1 mg/kg to about 500 mg/kg, of from greater than 5 mg/kg to about 500 mg/kg body weight of a second antiviral agent.

In certain embodiments, the antiviral agents are administered in a dose ratio in mg/kg of about 5:1, about 4:1, about 3:1, about 2:1, about 1:1, about 1:2, about 1:3, about 1:4, or about 1:5.

In certain embodiments, a composition or kit or device is provided whereby valacyclovir and famciclovir are administered together. In certain embodiments, valacyclovir and famciclovir are administered to the same subject in need thereof, by the same route or different routes, at the same time or at different times. In certain embodiments, the doses are relatively low doses, such as when the amount of valacyclovir is about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 0.2 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 0.3 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is 0.4 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 0.5 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 1 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 1.5 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 2 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 2 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 3 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 4 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 5 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 6 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 7 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 8 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 9 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 10 mg/kg, the amount of famciclovir can be 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 11 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 12.5 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 15 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 17.5 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 20 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 25 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 30 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 40 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacyclovir is about 50 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 60 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 70 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 80 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 90 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 100 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 125 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 150 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 175 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 200 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 225 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 250 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 275 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 300 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 325 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 350 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 375 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 400 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 425 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 450 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In an embodiment, when the amount of valacylovir is about 475 mg/kg, the amount of famciclovir can be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg.

In certain embodiments, it will be desirable for the dose total to be less than 500 mg/kg. For example, in embodiments wherein the dose ratio of valacyclovir:famciclovir is 2:1, non-limiting dosages include about 0.02 mg/kg:0.01 mg/kg, 0.03 mg/kg:0.015 mg/kg, 0.04 mg/kg: 0.02 mg/kg, 0.05 mg/kg:0.025 mg/kg, 0.06 mg/kg:0.03 mg/kg, 0.07 mg/kg:0.035 mg/kg, 0.08 mg/kg:0.04 mg/kg, 0.09 mg/kg:0.045 mg/kg, 0.1 mg/kg:0.05 mg/kg, 0.2 mg/kg:0.1 mg/kg, 0.4 mg/kg:0.2 mg/kg, 0.6 mg/kg:0.3 mg/kg, 0.8 mg/kg:0.4 mg/kg, 1 mg/kg:0.5 mg/kg, 1.2 mg/kg: 0.6 mg/kg, 1.4 mg/kg:0.7 mg/kg, 1.6 mg/kg:0.8 mg/kg, 1.8 mg/kg:0.9 mg/kg, 2 mg/kg:1 mg/kg, 4 mg/kg:2 mg/kg, 6 mg/kg:3 mg/kg, 8 mg/kg:4 mg/kg, 10 mg/kg:5 mg/kg, 12 mg/kg:6 mg/kg, 14 mg/kg:7 mg/kg, 16 mg/kg:8 mg/kg, 18 mg/kg:9 mg/kg, 20 mg/kg:10 mg/kg, 24 mg/kg:12 mg/kg, 28 mg/kg:14 mg/kg, 30 mg/kg:15 mg/kg, 32 mg/kg:16 mg, 36 mg/kg:18 mg, 40 mg/kg: 20 mg/kg, 50 mg/kg:25 mg/kg, 60 mg/kg:30 mg/kg, 80 mg/kg:40 mg/kg, 100 mg/kg:50 mg/kg, 120 mg/kg:60 mg/kg, 140 mg/kg:70 mg/kg, 160 mg/kg:80 mg/kg, 180 mg/kg:90 mg/kg, 200 mg/kg:100 mg/kg, 220 mg/kg:110 mg/kg, 240 mg/kg:120 mg/kg, 260 mg/kg:130 mg/kg, 280 mg/kg:140 mg/kg, 300 mg/kg:150 mg/kg.

An absolute dose or dose by body weight tested in an animal model can be a human equivalent dose (BED). See, e.g., NIH guidelines and Nair, A. B. et al., A simple practice guide for dose conversion between animals and human, J Basic Clin Pharm. March 2016-May 2016; 7(2): 27-31. Examples set forth herein using mice or guinea pigs employ human equivalent doses.

In certain embodiments, one or more antiviral agents used in a combination comprise herpesvirus polymerase inhibitors. In certain embodiments, one or more antiviral agents used in a combination comprise nucleoside analogs. Examples include, without limitation, FDA approved Famvir (famciclovir), Sitavig (acyclovir), Valtrex (valacyclovir), and Zovirax (acyclovir). In one embodiment, a combination of antiviral agents comprises valacyclovir and famciclovir. In another embodiment, a combination of antiviral agents comprises acyclovir and penciclovir. In certain embodiments, one or more antiviral agents used in a combination comprise HSV helicase-primase inhibitors (HPIs). Examples include, without limitation amenamevir and pritelivir. In certain embodiments, a combination comprises an HSV polymerase inhibitor and HSP helicase-primase inhibitor.

In certain embodiments, the first and second antiviral agents can comprise valacyclovir, acyclovir, famciclovir, penciclovir, pritelivir, penciclovir, ganciclovir, valganciclovir, cidofovir, foscarnet, darunavir, glycyrrhizic acid, Sambucus nigra, glutamine, FV-100, A5P2151, me-609, ASP2151, topical VDO, PEG-formulation (Devirex AG), vidarabine, cidofovir, crofelemer (SP-303T), EPB-348, CMX001, V212, NB-001, squaric acid, ionic zinc, sorivudine (ARYS-01), trifluridine, 882C87, merlin (ethanol and glycolic acid mixture), vitamin C, AIC316, versabase gel with Sarracenia purpurea, UB-621, lysine, edoxudine, brivudine, cytarabine, docosanol, tromantadine, resiquimod (R-848), imiquimod, resiquimod, tenofovir, tenofovir disoproxil fumarate, tenofovir alafenamide fumarate, amenamevir, N-methancocarbathymidine (N-MCT), or a salt, solvate or combination thereof.

Both valacyclovir and famciclovir have similar mechanisms of action in that they both target viral DNA. However, the drugs differ in their pharmacokinetic profile and specificity for viral thymidine kinase. It has been discovered that there is an unexpected and surprising synergy in the action of these drugs in combination. As both of these drugs are within the same class, it was not obvious to combine them. Indeed, it has been shown that increasing amounts of each drug, used individually in a monotherapy, does not increase the efficacy of the therapy. Since both are nucleoside analogs that both target viral DNA, the current recommendations from the CDC and other authorities are to treat herpes infections with either agent, not to employ both drugs in a combination therapy.

Valacyclovir is created with the addition of a valine moiety to acyclovir, greatly improving bioavailability and resulting in a substrate for active transport mechanisms in the human intestine through the peptide transporters hPEPT-1 and/or HPT-1. There is minimal protein binding (<15%), and satisfactory distribution into body tissues including the cerebral spinal fluid (i.e., CSF). Acyclovir is phosphorylated primarily by viral thymidine kinases that create a concentration gradient that increases its uptake into infected cells. Without viral thymidine kinases, acyclovir is eliminated, basically unchanged, via tubular secretion and glomerular filtration. This is the main reason for valacyclovir's superior safety profile. It is also not metabolized by cytochrome p450, thus limiting drug interactions. The mechanism of action is threefold: 1) competitive inhibition of viral DNA polymerase; 2) chain termination of viral DNA; and 3) inactivation of viral DNA polymerase. The half-life is approximately 2.5-3.4 hours. Mean Cmax ranges from 0.83 to 5.65 ug/mL. Mean AUC ranged from 2.27-19.62 hr*ug/mL. Renal clearance following a single 1 g dose was 255 mL/min. Valacyclovir has overall bioavailability of 54.5% compared to FCV 77%.

Famciclovir has a weaker affinity for viral DNA polymerase, a shorter plasma half-life (2-2.5 hr), but has a longer intracellular half-life (10-20 hours in HSV infected cells), as well as high-intracellular concentrations versus valacyclovir. The time to reach maximum concentration ranges from 0.5-0.75 hours versus 1.6-1.9 hrs for valacyclovir. Famciclovir also has high bioavailability at 77%. Famciclovir does not use peptide transporters hPEPT-1 or HPT-1, and thus should be more efficacious in subjects who have defects related to these transporters.

The combination of valacyclovir and famciclovir is useful for the prevention of human herpesvirus infections (i.e., pre-exposure prophylaxis) as well as the treatment and prevention of recurrences of symptoms due to herpesviruses (i.e., post-exposure prophylaxis). The combination of valacyclovir and famciclovir is useful for the prevention of Equine Herpesvirus infections (i.e., pre-exposure prophylaxis) as well as the treatment and prevention of recurrences of symptoms due to herpesviruses (i.e., post-exposure prophylaxis). The combination is nontoxic at doses producing potent antiviral activity, and is active and bioavailable by the oral or parenteral route of administration.

Another nucleoside inhibitor of herpesvirus polymerase is N-methanocarbathymidine (N-MCT), which is converted into the active triphosphate metabolite by a three step phosphorylation process, one step of which is selectively accomplished by HSV-TK. N-MCT is inhibits HSV-1, HSV-2, HSV-8 (Kaposi's Sarcoma Virus), as well as Vaccinia and Cowpox viruses, is cytotoxic only in virus-infected cells, is active against acyclovir-resistant HSV and cidofovir-resistant cowpox virus, is equally or more potent than the antiviral drugs acyclovir, ganciclovir and cidofovir, is nontoxic at doses producing potent antiviral activity, and is active and bioavailable by the oral route or parenteral of administration.

In some embodiments, the first antiviral agent comprises a herpesvirus vaccine and optionally an adjuvant. In some embodiments, the herpesvirus vaccine comprises GSK208141 (gD2t, GSK glycoprotein D (gD)-Alum/3-deacylated form of monophosphoryl lipid A), Herpes Zoster GSK 1437173A, gD2-A504, Havrix™, gD-Alum, Zostavax/Zoster vaccine (V211, V212, V210), HSV529, HerpV (AG-707 rh-Hsc70 polyvalent peptide complex), VCL-HBO1, VCL-HMO1, pPJV7630, GEN-003, 5PL7013 gel (VivaGel™), G5K324332A, or a salt, solvate or combination thereof.

Non-limiting examples of herpesvirus vaccines useful as antiviral agents in compositions and methods described herein include GSK208141 (gD2t, GSK glycoprotein D (gD)-Alum/3-deacylated form of monophosphoryl lipid A), Herpes Zoster GSK 1437173A, gD2-ASO4, Havrix™, gD-Alum, Zostavax/Zoster vaccine (V211, V212, V210), HSV529, HerpV (AG-707 rh-Hsc70 polyvalent peptide complex), VCL-HBO1, VCL-HM01, pPJV7630, GEN-003, SPL7013 gel (VivaGel™) GSK324332A, and salts, solvates, and/or combinations thereof.

In some embodiments, an antiviral agent inhibits viral entry and/or viral fusion. Examples of entry and/or fusion inhibitors include, without limitation, maraviroc, enfuvirtide (T-20), vicriviroc, cenicriviroc, lbalizumab, fostemsavir (BMS-663068), ibalizumab (TMB-355, TNX-355), PRO 140, b12 antibody, DCM205, DARPins, caprine antibody, VIR-576, AMD11070, PR0542, SCH-C, T-1249, cyanovirin, griffithsen, lectins, pentafuside, and salts, solvates, and/or combinations thereof.

In some embodiments, an antiviral agent is an integrase inhibitor. Non-limiting examples of integrase inhibitors include dolutegravir, elvitegravir, raltegravir, globoidnan A, MK-2048, BI224436, cabotegravir, GSK 1265744, GSK-572, MK-0518, and salts, solvates, or combinations thereof.

In some embodiments, an antiviral agent is a reverse transcriptase inhibitor, for example, a nucleotide reverse transcriptase inhibitor and/or nucleoside reverse transcriptase inhibitor (NRTI). In some embodiments, an antiviral agent is a non-nucleoside reverse transcriptase inhibitor (NNRTI). NRTIs include, without limitation, abacavir, didanosine, emtrictabine, lamivudine, stavudine, tenofovir, tenofovir disoporoxil fumarate, zidovudine, apricitabine, stampidine, elvucitabine, racivir, amdoxovir, stavudine, zalcitabine, festinavir, dideoxycytidine ddC, azidothymidine, tenofovir alafenamide fumarate, entecavir, and salts, solvates, and/or combinations thereof. NNRTIs include, without limitation, delavirdine, efavirenz, etravirine (TMC-125), nevirapine, rilpivirine, doravirine, Calanolide A, capravirine, epivir, adefovir, dapivirine, lersivirine, and salts, solvates, and/or combinations thereof. Further non-limiting examples of reverse transcriptase inhibitors include alovudine, elvucitabine, TMC-278, DPC-083, amdoxovir, (−)-beta-D-2,6-diamino-purine dioxolane, MIV-210 (FLG), DFC (dexelvucitabine), dioxolane thymidine, L697639, atevirdine (U87201E), MIV-150, GSK-695634, GSK-678248, TMC-278, KP1461, KP-1212, lodenosine (FddA), 5-[(3,5-dichlorophenyl)thio]-4-isopropyl-1-(4-pyridylmethyl)imidazole-2-methanol carbamic acid, (−)-I2-D-2,6-diaminopurine dioxolane, AVX-754, BCH-13520, BMS-56190 ((4S)-6-chloro-4-[(1E)-cyclopropylethenyl]-3,-4-dihydro-4-trifluoromethyl-2(1H)-quinazolinone), TMC-120, L697639, and salts, solvates, and/or combinations thereof.

In some embodiments, an antiviral agent is a protease inhibitor. Non-limiting examples of protease inhibitors include atazanavir, darunavir, fosamprenavir, indinavir, nelfinavir, ritonavir, saquinavir, tipranavir, lopinavir, amprenavir, telinavir (SC-52151), droxinavir, emtriva, invirase, agenerase, TMC-126, mozenavir (DMP-450), JE-2147 (AG1776), L-756423, KNI-272, DPC-681, DPC-684, BMS 186318, droxinavir (SC-55389a), DMP-323, KNI-227, 1-[(2-hydroxyethoxy)methyl]-6-(phenylthio)-thymine, AG-1859, RO-033-4649, R-944, DMP-850, DMP-851, brecanavir (GW640385), and salts, solvates, and/or combinations thereof.

In some embodiments, an antiviral agent is a microbicide. Non-limiting examples of microbicides include detergents such as nonoxynol-9, sodium dodecyl sulfate and Savvy (1.0% C31G). In some embodiments, the microbicide reduces the pH of vaginal secretions, for example, BufferGel®. In some embodiments, an antiviral agent comprises a polyanion microbicide. Examples of polyanion microbicides are carrageenans. Carrageenans are linear sulfated polysaccharides which can be used by viruses for initial attachment to a cell membrane, thus acting as decoy receptors for viral binding. In some embodiments, a microbicide is a nanoscale dendrimeric molecule which binds to a virus. An example of a nanoscale dendrimeric molecule is the active component of VivaGel®. An additional example of a microbicide is PRO-2000, also known as PRO 2000/5, naphthalene 2-sulfonate polymer, or polynaphthalene sulphonate.

In some embodiments, a composition comprises an active agent that is used as an antiviral “off-label” agent for the treatment or prevention of viral infection. In some cases, a composition is administered with an “off-label” agent. Non-limiting examples of “off-label” agents useful with or in the compositions provided herein include amphotericin B, sulfamethoxazole, trimethoprim, clarithromycin, daunorubicin, fluconazole, doxorubicin, anidulafungin, immune globulin, gamma globulin, dronabinol, megestrol acetate, atovaquone, rifabutin, pentamidine, trimetrexate glucuronate, leucovorin, alitretinoin gel, erythropoietin, calcium hydroxyapatite, poly-L-lactic acid, somatropin rDNA, itraconazole, paclitaxel, voriconazole, cidofovir, fomivirsen, azithromycin, and salts, solvates, and/or combinations thereof. In some embodiments, an antiviral agent is a cancer agent, for example, ruxolitinib or tocilizumab.

In some embodiments, a composition for preventing or treating a viral infection comprises at least one of the following antiviral agents: bevirimat, TRIM5alpha, Tat antagonists, trichosanthin, abzyme, calanolide A, ceragenin, cyanovirin-N, diarylpyrimidines, epigallocatechin gallate (EGCG), foscarnet, griffithsin, hydroxycarbamide, miltefosine, portmanteau inhibitors, scytovirin, seliciclib, synergistic enhancers, tre recombinase, zinc finger protein transcription factor, KP-1461, BIT225, aplaviroc, atevirdine, brecanavir, capravirine, dexelvucitabine, emivirine, lersivirine, lodenosine, loviride, fomivirsen, glycyrrhizic acid (anti-inflammatory, inhibits 1 lbeta-hydroxysteroid dehydrogenase), zinc salts, cellulose sulfate, cyclodextrins, dextrin-2-sulfate, NCP7 inhibitors, AMD-3100, BMS-806, BMS-793, C31G, carrageenan, CD4-IgG2, cellulose acetate phthalate, mAb 2G12, mAb b12, Merck 167, plant lectins, poly naphthalene sulfate, poly sulfo-styrene, PRO2000, PSC-Rantes, SCH-C, SCH-D, T-20, TMC-125, UC-781, UK-427, UK-857, C5A, or salts, solvates, or combinations thereof.

In some embodiments, a composition for preventing or treating a viral infection comprises at least one of the following antiviral agents: Carraguard (PC-515), brincidofovir (CMX001), zidovudine, virus-specific cytotoxic T cells, idoxuridine, podophyllotoxin, rifampicin, metisazone, interferon alfa 2b (Intron-A), peginterferon alfa-2a, ribavirin, moroxydine, pleconaril, BCX4430, taribavirin (viramidine, ICN 3142), favipiravir, rintatolimod, ibacitabine, (5-iodo-2′-deoxycytidine), methisazone (metisazone), ampligen, arbidol, Atripla®, combivir, imunovir, nexavir, trizivir, truvada, lamivudine, dideoxyadenosine, floxuridine, idozuridine, inosine pranobex, 2′-deoxy-5-(methylamino)uridine, digoxin, imiquimod, interferon type III, interferon type II, interferon type I, tea tree oil, or salts, solvates, or combinations thereof.

In some embodiments, a composition for preventing or treating a viral infection comprises a hepatitis antiviral agent comprising tenofovir, glycyrrhizic acid, fialuridine, telbivudine, adefovir, etecavir, lamivudine, clevudine, asunaprevir, boceprevir, faldaprevir, grazoprevir, paritaprevir, ritonavir, telaprevir, simeprevir, sofosbuvir, ACH-3102, daclatasvir, deleobuvir, elbasvir, ledipasvir, MK-3682, MK-8408, samatasvir, ombitasvir, entecavir, or salts, solvates, or combinations thereof.

In some embodiments, a composition for preventing or treating a viral infection comprises an influenza antiviral comprising elderberry sambucus, umifenovir, amantadine, rimantadine, oseltamivir, zanamivir, peramivir, laninamivir, or salts, solvates, or combinations thereof.

In some embodiments, a composition for preventing or treating a viral infection comprises an HPV antiviral comprising pyrrole polyamides, lopinavir, carrageenan, zinc, or salts, solvates, or combinations thereof.

In some aspects, provided are compositions comprising an antiviral agent in combination with at least one additional active agent, wherein a combination refers to a composition comprising a combination of two or more active agents or a composition comprising an antiviral agent administered in combination with one or more additional active agents. In some cases, a combination of active agents results in a synergistic effect in antiviral activity. A synergistic effect may be calculated, in one example, using the Sigmoid-Emax, Loewe, and median-effect equations.

In some embodiments, a composition comprising an antiviral agent useful for the prevention and/or treatment of a viral disease further comprises or is administered in combination with another agent or active agent for the prevention and/or treatment of the viral disease. In some cases, the composition comprises or is administered with a vaccine, gene therapy treatment, cytokine, TAT inhibitor, immunomodulator or combinations thereof. In some cases, the composition comprises or is administered with an anti-infective agent comprising an antifungal agent, an antibacterial, an antiprotozoal agent or combinations thereof. In some cases, the composition comprises or is administered with an immunomodulator, for example, pentamidine isethionate, autologous CD8+ infusion, gamma-interferon immunoglobulins, thymic peptides, IGF-I, anti-Leu3A, auto vaccination, biostimulation, extracorporeal photophoresis, cyclosporin, rapamycin, FK-565, FK-506, GCSF, GM-CSF, hyperthermia, isopinosine, rVIG, HIVIG, passive immunotherapy and polio vaccine hyperimmunization, or combinations thereof. In some embodiments, a composition comprises or is administered with a pharmacokinetic enhancer, for example, cobicistat. In some embodiments, a composition comprises or is administered with a synergistic enhancer, for example, chloroquine/quinoline enhancers of protease inhibitors, CYP3A4, hydroxyurea, leflunomide, mycophenolic acid, resveratrol, or combinations thereof.

In some embodiments, a composition useful in the methods described herein comprises a combination of active agents, at least one of which is an antiviral agent. Examples of combinations include, without limitation, maravoric and emtricitabine; maravoric, emtricitabine and raltegravir; raltegravir and emtricitabine; raltegravir and lamivudine; abacavir and lamivudine; abacavir, dolutegravir, and lamivudine; abacavir, lamivudine, and zidovudine; atazanavir and cobicistat; darunavir and cobicistat; efavirenz, emtricitabine, and tenofovir; elvitegravir, cobicistat, emtricitabine, and tenofovir; emtricitabine, rilpivirine, and tenofovir; lamivudine and raltegravir; lamivudine and zidovudine; lopinavir and ritonavir; emtricitabine and tenofovir disoproxil fumarate; lamivudine and zidovudine; lopinavir and ritonavir; inosine, acetamidobenzoic acid and dimethylaminoisopropanol; and salts, solvates, and/or combinations thereof.

In some embodiments, a composition useful in the methods described herein for the prevention of viral infection (e.g., herpesvirus) in a seronegative subject and/or the treatment of a viral infection (e.g., herpesvirus suppression treatment) in a seropositive subject comprises one or more of the following active agents: valacyclovir, acyclovir, famciclovir, pritelivir, penciclovir, ganciclovir, valganciclovir, cidofovir, foscarnet, darunavir, glycyrrhizic acid, glutamine, FV-100, ASP2151, me-609, ASP2151, topical VDO, PEG-formulation (Devirex AG), vidarabine, cidofovir, crofelemer (SP-303T), EPB-348, CMX001, V212, NB-001, squaric acid, ionic zinc, sorivudine (ARYS-01), trifluridine, 882C87, merlin (ethanol and glycolic acid mixture), vitamin C, AIC316, versabase gel with Sarracenia purpurea, UB-621, lysine, edoxudine, brivudine, cytarabine, docosanol, tromantadine, resiquimod (R-848), imiquimod, resiquimod, tenofovir, tenofovir disoproxil fumarate, tenofovir alafenamide fumarate, include GSK208141 (gD2t, GSK glycoprotein D (gD)-Alum/3-deacylated form of monophosphoryl lipid A), Herpes Zoster GSK 1437173A, gD2-ASO4, Havrix™ gD-Alum, Zostavax/Zoster vaccine (V211, V212, V210), HSV529, HerpV (AG-707 rh-Hsc70 polyvalent peptide complex), VCL-HB01, VCL-HMO1, pPJV7630, GEN-003, SPL7013 gel (VivaGel™), GSK324332A, GSK1492903A, VariZIG™, and Varivax, maraviroc, enfuvirtide, vicriviroc, cenicriviroc, lbalizumab, fostemsavir (BMS-663068), ibalizumab (TMB-355, TNX-355), PRO 140, b12 antibody, DCM205, DARPins, caprine antibody, VIR-576, enfuvirtide (T-20), AMD11070, PR0542, SCH-C, T-1249, cyanovirin, griffithsen, lectins, pentafuside, dolutegravir, elvitegravir, raltegravir, globoidnan A, MK-2048, B1224436, cabotegravir, GSK 1265744, GSK-572, MK-0518, abacavir, didanosine, emtrictabine, lamivudine, stavudine, tenofovir, tenofovir disoporoxil fumarate, zidovudine, apricitabine, stampidine, elvucitabine, racivir, amdoxovir, stavudine, zalcitabine, festinavir, dideoxycytidine ddC, azidothymidine, tenofovir alafenamide fumarate, entecavir, delavirdine, efavirenz, etravirine (TMC-125), nevirapine, rilpivirine, doravirine, Calanolide A, capravirine, epivir, adefovir, dapivirine, lersivirine, alovudine, elvucitabine, TMC-278, DPC-083, amdoxovir, (−)-beta-D-2,6-diamino-purine dioxolane, MIV-210 (FLG), DFC (dexelvucitabine), dioxolane thymidine, L697639, atevirdine (U87201E), MIV-150, GSK-695634, GSK-678248, TMC-278, KP1461, KP-1212, lodenosine (FddA), 5-[(3,5-dichlorophenyl)thio]-4-isopropyl-1-(4-pyridylmethyl)imidazole-2-methanol carbamic acid, (−)-I2-D-2,6-diaminopurine dioxolane, AVX-754, BCH-13520, BMS-56190 ((4S)-6-chloro-4-[(1E)-cyclopropylethenyl]-3,-4-dihydro-4-trifluoromethyl-2(1H)-quinazolinone), TMC-120, L697639, atazanavir, darunavir, fosamprenavir, indinavir, nelfinavir, ritonavir, saquinavir, tipranavir, lopinavir, amprenavir, telinavir (SC-52151), droxinavir, emtriva, invirase, agenerase, TMC-126, mozenavir (DMP-450), JE-2147 (AG1776), L-756423, KNI-272, DPC-681, DPC-684, BMS 186318, droxinavir (SC-55389a), DMP-323, KNI-227, 1-[(2-hydroxyethoxy)methyl]-6-(phenylthio)-thymine, AG-1859, RO-033-4649, R-944, DMP-850, DMP-851, brecanavir (GW640385), nonoxynol-9, sodium dodecyl sulfate, Savvy (1.0% C31G), BufferGel®, carrageenans, VivaGel®, PRO-2000, also known as PRO 2000/5, naphthalene 2-sulfonate polymer, or polynaphthalene sulphonate, amphotericin B, sulfamethoxazole, trimethoprim, clarithromycin, daunorubicin, fluconazole, doxorubicin, anidulafungin, immune globulin, gamma globulin, dronabinol, megestrol acetate, atovaquone, rifabutin, pentamidine, trimetrexate glucuronate, leucovorin, alitretinoin gel, erythropoietin, calcium hydroxyapatite, poly-L-lactic acid, somatropin rDNA, itraconazole, paclitaxel, voriconazole, cidofovir, fomivirsen, azithromycin, ruxolitinib, tocilizumab, bevirimat, TRIM5alpha, Tat antagonists, trichosanthin, abzyme, calanolide A, ceragenin, cyanovirin-N, diarylpyrimidines, epigallocatechin gallate (EGCG), foscarnet, griffithsin, hydroxycarbamide, miltefosine, portmanteau inhibitors, scytovirin, seliciclib, synergistic enhancers, tre recombinase, zinc finger protein transcription factor, KP-1461, BIT225, aplaviroc, atevirdine, brecanavir, capravirine, dexelvucitabine, emivirine, lersivirine, lodenosine, loviride, fomivirsen, glycyrrhizic acid (anti-inflammatory, inhibits 1 lbeta-hydroxysteroid dehydrogenase), zinc salts, cellulose sulfate, cyclodextrins, dextrin-2-sulfate, NCP7 inhibitors, AMD-3100, BMS-806, BMS-793, C31G, carrageenan, CD4-IgG2, cellulose acetate phthalate, mAb 2G12, mAb b12, Merck 167, plant lectins, poly naphthalene sulfate, poly sulfo-styrene, PRO2000, PSC-Rantes, SCH-C, SCH-D, T-20, TMC-125, UC-781, UK-427, UK-857, Carraguard (PC-515), brincidofovir (CMX001), zidovudine, virus-specific cytotoxic T cells, idoxuridine, podophyllotoxin, rifampicin, metisazone, interferon alfa 2b (Intron-A), peginterferon alfa-2a, ribavirin, moroxydine, pleconaril, BCX4430, taribavirin (viramidine, ICN 3142), favipiravir, rintatolimod, ibacitabine, (5-iodo-2′-deoxycytidine), methisazone (metisazone), ampligen, arbidol, Atripla®, combivir, imunovir, nexavir, trizivir, truvada, lamivudine, dideoxyadenosine, floxuridine, idozuridine, inosine pranobex, 2′-deoxy-5-(methylamino)uridine, digoxin, imiquimod, interferon type III, interferon type II, interferon type I, tea tree oil, glycyrrhizic acid, fialuridine, telbivudine, adefovir, etecavir, lamivudine, clevudine, asunaprevir, boceprevir, faldaprevir, grazoprevir, paritaprevir, ritonavir, telaprevir, simeprevir, sofosbuvir, ACH-3102, daclatasvir, deleobuvir, elbasvir, ledipasvir, MK-3682, MK-8408, samatasvir, ombitasvir, entecavir, elderberry sambucus, umifenovir, amantadine, rimantadine, oseltamivir, zanamivir, peramivir, laninamivir, pyrrole polyamides, lopinavir, or salts, solvates, and/or combinations thereof.

In some embodiments, a composition useful in the methods described herein for the prevention of herpesvirus infection in a seronegative subject and/or the treatment of herpesvirus in a seropositive subject comprises one or more of the following antivirals: valacyclovir, acyclovir, famciclovir, pritelivir, penciclovir, ganciclovir, valganciclovir, cidofovir, foscarnet, darunavir, glycyrrhizic acid, glutamine, FV-100, ASP2151, me-609, ASP2151, topical VDO, PEG-formulation (Devirex AG), vidarabine, cidofovir, crofelemer (SP-303T), EPB-348, CMX001, V212, NB-001, squaric acid, ionic zinc, sorivudine (ARYS-01), trifluridine, 882C87, merlin (ethanol and glycolic acid mixture), vitamin C, AIC316, versabase gel with Sarracenia purpurea, UB-621, lysine, edoxudine, brivudine, cytarabine, docosanol, tromantadine, resiquimod (R-848), imiquimod, resiquimod, tenofovir, tenofovir disoproxil fumarate, tenofovir alafenamide fumarate, C5A or salts, solvates, or combinations thereof.

In some embodiments, a composition useful in the methods described herein for the prevention of viral infection (e.g., herpesvirus) in a seronegative subject and/or the treatment of viral infection (e.g., herpesvirus suppression treatment) in a seropositive subject comprises 2, 3, 4, 5 or more of the following active agents valacyclovir, acyclovir, famciclovir, pritelivir, penciclovir, ganciclovir, valganciclovir, cidofovir, foscarnet, darunavir, glycyrrhizic acid, glutamine, FV-100, ASP2151, me-609, ASP2151, topical VDO, PEG-formulation (Devirex AG), vidarabine, cidofovir, crofelemer (SP-303T), EPB-348, CMX001, V212, NB-001, squaric acid, ionic zinc, sorivudine (ARYS-01), trifluridine, 882C87, merlin (ethanol and glycolic acid mixture), vitamin C, AIC316, versabase gel with Sarracenia purpurea, UB-621, lysine, edoxudine, brivudine, cytarabine, docosanol, tromantadine, resiquimod (R-848), imiquimod, resiquimod, tenofovir, tenofovir disoproxil fumarate, tenofovir alafenamide fumarate, include GSK208141 (gD2t, GSK glycoprotein D (gD)-Alum/3-deacylated form of monophosphoryl lipid A), Herpes Zoster GSK 1437173A, gD2-ASO4, Havrix™ gD-Alum, Zostavax/Zoster vaccine (V211, V212, V210), HSV529, HerpV (AG-707 rh-Hsc70 polyvalent peptide complex), VCL-HB01, VCL-HM01, pPJV7630, GEN-003, SPL7013 gel (VivaGel™), GSK324332A, GSK1492903A, VariZIG™, and Varivax, maraviroc, enfuvirtide, vicriviroc, cenicriviroc, lbalizumab, fostemsavir (BMS-663068), ibalizumab (TMB-355, TNX-355), PRO 140, b12 antibody, DCM205, DARPins, caprine antibody, VIR-576, enfuvirtide (T-20), AMD11070, PR0542, SCH-C, T-1249, cyanovirin, griffithsen, lectins, pentafuside, dolutegravir, elvitegravir, raltegravir, globoidnan A, MK-2048, BI224436, cabotegravir, GSK 1265744, GSK-572, MK-0518, abacavir, didanosine, emtrictabine, lamivudine, stavudine, tenofovir, tenofovir disoporoxil fumarate, zidovudine, apricitabine, stampidine, elvucitabine, racivir, amdoxovir, stavudine, zalcitabine, festinavir, dideoxycytidine ddC, azidothymidine, tenofovir alafenamide fumarate, entecavir, delavirdine, efavirenz, etravirine (TMC-125), nevirapine, rilpivirine, doravirine, Calanolide A, capravirine, epivir, adefovir, dapivirine, lersivirine, alovudine, elvucitabine, TMC-278, DPC-083, amdoxovir, (−)-beta-D-2,6-diamino-purine dioxolane, MIV-210 (FLG), DFC (dexelvucitabine), dioxolane thymidine, L697639, atevirdine (U87201E), MIV-150, GSK-695634, GSK-678248, TMC-278, KP1461, KP-1212, lodenosine (FddA), 5-[(3,5-dichlorophenyl)thio]-4-isopropyl-1-(4-pyridylmethyl)imidazole-2-methanol carbamic acid, (−)-I2-D-2,6-diaminopurine dioxolane, AVX-754, BCH-13520, BMS-56190 ((4S)-6-chloro-4-[(1E)-cyclopropylethenyl]-3,-4-dihydro-4-trifluoromethyl-2(1H)-quinazolinone), TMC-120, L697639, atazanavir, darunavir, fosamprenavir, indinavir, nelfinavir, ritonavir, saquinavir, tipranavir, lopinavir, amprenavir, telinavir (SC-52151), droxinavir, emtriva, invirase, agenerase, TMC-126, mozenavir (DMP-450), JE-2147 (AG1776), L-756423, KNI-272, DPC-681, DPC-684, BMS 186318, droxinavir (SC-55389a), DMP-323, KNI-227, 1-[(2-hydroxyethoxy)methyl]-6-(phenylthio)-thymine, AG-1859, RO-033-4649, R-944, DMP-850, DMP-851, brecanavir (GW640385), nonoxynol-9, sodium dodecyl sulfate, Savvy (1.0% C31G), BufferGel®, carrageenans, VivaGel®, PRO-2000, also known as PRO 2000/5, naphthalene 2-sulfonate polymer, or polynaphthalene sulphonate, amphotericin B, sulfamethoxazole, trimethoprim, clarithromycin, daunorubicin, fluconazole, doxorubicin, anidulafungin, immune globulin, gamma globulin, dronabinol, megestrol acetate, atovaquone, rifabutin, pentamidine, trimetrexate glucuronate, leucovorin, alitretinoin gel, erythropoietin, calcium hydroxyapatite, poly-L-lactic acid, somatropin rDNA, itraconazole, paclitaxel, voriconazole, cidofovir, fomivirsen, azithromycin, ruxolitinib, tocilizumab, bevirimat, TRIM5alpha, Tat antagonists, trichosanthin, abzyme, calanolide A, ceragenin, cyanovirin-N, diarylpyrimidines, epigallocatechin gallate (EGCG), foscarnet, griffithsin, hydroxycarbamide, miltefosine, portmanteau inhibitors, scytovirin, seliciclib, synergistic enhancers, tre recombinase, zinc finger protein transcription factor, KP-1461, BIT225, aplaviroc, atevirdine, brecanavir, capravirine, dexelvucitabine, emivirine, lersivirine, lodenosine, loviride, fomivirsen, glycyrrhizic acid (anti-inflammatory, inhibits 1 lbeta-hydroxysteroid dehydrogenase), zinc salts, cellulose sulfate, cyclodextrins, dextrin-2-sulfate, NCP7 inhibitors, AMD-3100, BMS-806, BMS-793, C31G, carrageenan, CD4-IgG2, cellulose acetate phthalate, mAb 2G12, mAb b12, Merck 167, plant lectins, poly naphthalene sulfate, poly sulfo-styrene, PRO2000, PSC-Rantes, SCH-C, SCH-D, T-20, TMC-125, UC-781, UK-427, UK-857, Carraguard (PC-515), brincidofovir (CMX001), zidovudine, virus-specific cytotoxic T cells, idoxuridine, podophyllotoxin, rifampicin, metisazone, interferon alfa 2b (Intron-A), peginterferon alfa-2a, ribavirin, moroxydine, pleconaril, BCX4430, taribavirin (viramidine, ICN 3142), favipiravir, rintatolimod, ibacitabine, (5-iodo-2′-deoxycytidine), methisazone (metisazone), ampligen, arbidol, Atripla®, combivir, imunovir, nexavir, trizivir, truvada, lamivudine, dideoxyadenosine, floxuridine, idozuridine, inosine pranobex, 2′-deoxy-5-(methylamino)uridine, digoxin, imiquimod, interferon type III, interferon type II, interferon type I, tea tree oil, glycyrrhizic acid, fialuridine, telbivudine, adefovir, etecavir, lamivudine, clevudine, asunaprevir, boceprevir, faldaprevir, grazoprevir, paritaprevir, ritonavir, telaprevir, simeprevir, sofosbuvir, ACH-3102, daclatasvir, deleobuvir, elbasvir, ledipasvir, MK-3682, MK-8408, samatasvir, ombitasvir, entecavir, elderberry sambucus, umifenovir, amantadine, rimantadine, oseltamivir, zanamivir, peramivir, laninamivir, pyrrole polyamides, lopinavir, C5A or salts, solvates, and/or combinations thereof.

In some embodiments, a composition useful in the methods described herein for the prevention of herpesvirus infection in a seronegative subject and/or the treatment of herpesvirus in a seropositive subject comprises 2, 3, 4, 5 or more of the following antiviral agents: valacyclovir, acyclovir, famciclovir, pritelivir, penciclovir, ganciclovir, valganciclovir, cidofovir, foscarnet, darunavir, glycyrrhizic acid, glutamine, FV-100, ASP2151, me-609, ASP2151, topical VDO, PEG-formulation (Devirex AG), vidarabine, cidofovir, crofelemer (SP-303T), EPB-348, CMX001, V212, NB-001, squaric acid, ionic zinc, sorivudine (ARYS-01), trifluridine, 882C87, merlin (ethanol and glycolic acid mixture), vitamin C, AIC316, versabase gel with Sarracenia purpurea, UB-621, lysine, edoxudine, brivudine, cytarabine, docosanol, tromantadine, resiquimod (R-848), imiquimod, resiquimod, tenofovir, tenofovir disoproxil fumarate, tenofovir alafenamide fumarate, C5A or salts, solvates, or combinations thereof.

In some embodiments, a composition useful in the methods described herein for the prevention of viral infection (e.g., herpesvirus) in a seronegative subject and/or the treatment of viral infection (e.g., herpesvirus suppression treatment) in a seropositive subject comprises one or more of the following active agents: valacyclovir, acyclovir, famciclovir, pritelivir, penciclovir, ganciclovir, valganciclovir, cidofovir, foscarnet, darunavir, glycyrrhizic acid, glutamine, FV-100, ASP2151, me-609, ASP2151, topical VDO, PEG-formulation (Devirex AG), vidarabine, cidofovir, crofelemer (SP-303T), EPB-348, CMX001, V212, NB-001, squaric acid, ionic zinc, sorivudine (ARYS-01), trifluridine, 882C87, merlin (ethanol and glycolic acid mixture), vitamin C, AIC316, versabase gel with Sarracenia purpurea, UB-621, lysine, edoxudine, brivudine, cytarabine, docosanol, tromantadine, resiquimod (R-848), imiquimod, resiquimod, tenofovir, tenofovir disoproxil fumarate, tenofovir alafenamide fumarate, or salts, solvates or combinations thereof, and one or more of the following active agents: maraviroc, enfuvirtide, vicriviroc, cenicriviroc, lbalizumab, fostemsavir (BMS-663068), ibalizumab (TMB-355, TNX-355), PRO 140, b12 antibody, DCM205, DARPins, caprine antibody, VIR-576, enfuvirtide (T-20), AMD11070, PR0542, SCH-C, T-1249, cyanovirin, griffithsen, lectins, pentafuside, dolutegravir, elvitegravir, raltegravir, globoidnan A, MK-2048, B1224436, cabotegravir, GSK 1265744, GSK-572, MK-0518, abacavir, didanosine, emtrictabine, lamivudine, stavudine, tenofovir, tenofovir disoporoxil fumarate, zidovudine, apricitabine, stampidine, elvucitabine, racivir, amdoxovir, stavudine, zalcitabine, festinavir, dideoxycytidine ddC, azidothymidine, tenofovir alafenamide fumarate, entecavir, delavirdine, efavirenz, etravirine (TMC-125), nevirapine, rilpivirine, doravirine, Calanolide A, capravirine, epivir, adefovir, dapivirine, lersivirine, alovudine, elvucitabine, TMC-278, DPC-083, amdoxovir, (−)-beta-D-2,6-diamino-purine dioxolane, MIV-210 (FLG), DFC (dexelvucitabine), dioxolane thymidine, L697639, atevirdine (U87201E), MIV-150, GSK-695634, GSK-678248, TMC-278, KP1461, KP-1212, lodenosine (FddA), 5-[(3,5-dichlorophenyl)thio]-4-isopropyl-1-(4-pyridylmethyl)imidazole-2-methanol carbamic acid, (−)-I2-D-2,6-diaminopurine dioxolane, AVX-754, BCH-13520, BMS-56190 ((4S)-6-chloro-4-[(1E)-cyclopropylethenyl]-3,-4-dihydro-4-trifluoromethyl-2(1H)-quinazolinone), TMC-120, L697639, atazanavir, darunavir, fosamprenavir, indinavir, nelfinavir, ritonavir, saquinavir, tipranavir, lopinavir, amprenavir, telinavir (SC-52151), droxinavir, emtriva, invirase, agenerase, TMC-126, mozenavir (DMP-450), JE-2147 (AG1776), L-756423, KNI-272, DPC-681, DPC-684, BMS 186318, droxinavir (SC-55389a), DMP-323, KNI-227, 1-[(2-hydroxyethoxy)methyl]-6-(phenylthio)-thymine, AG-1859, RO-033-4649, R-944, DMP-850, DMP-851, brecanavir (GW640385), or salts, solvates, or combinations thereof.

In some embodiments, a composition useful in the methods described herein for the prevention of herpesvirus infection in a seronegative subject and/or the treatment of herpesvirus in a seropositive subject comprises 2, 3, 4, 5 or more of the following active agents: valacyclovir, acyclovir, famciclovir, pritelivir, penciclovir, ganciclovir, valganciclovir, cidofovir, foscarnet, darunavir, glycyrrhizic acid, glutamine, FV-100, ASP2151, me-609, ASP2151, topical VDO, PEG-formulation (Devirex AG), vidarabine, cidofovir, crofelemer (SP-303T), EPB-348, CMX001, V212, NB-001, squaric acid, ionic zinc, sorivudine (ARYS-01), trifluridine, 882C87, merlin (ethanol and glycolic acid mixture), vitamin C, AIC316, versabase gel with Sarracenia purpurea, UB-621, lysine, edoxudine, brivudine, cytarabine, docosanol, tromantadine, resiquimod (R-848), imiquimod, resiquimod, tenofovir, tenofovir disoproxil fumarate, tenofovir alafenamide fumarate, or salts, solvates, or combinations thereof; and 2, 3, 4, 5 or more of the following active agents: maraviroc, enfuvirtide, vicriviroc, cenicriviroc, lbalizumab, fostemsavir (BMS-663068), ibalizumab (TMB-355, TNX-355), PRO 140, b12 antibody, DCM205, DARPins, caprine antibody, VIR-576, enfuvirtide (T-20), AMD11070, PR0542, SCH-C, T-1249, cyanovirin, griffithsen, lectins, pentafuside, dolutegravir, elvitegravir, raltegravir, globoidnan A, MK-2048, B1224436, cabotegravir, GSK 1265744, GSK-572, MK-0518, abacavir, didanosine, emtrictabine, lamivudine, stavudine, tenofovir, tenofovir disoporoxil fumarate, zidovudine, apricitabine, stampidine, elvucitabine, racivir, amdoxovir, stavudine, zalcitabine, festinavir, dideoxycytidine ddC, azidothymidine, tenofovir alafenamide fumarate, entecavir, delavirdine, efavirenz, etravirine (TMC-125), nevirapine, rilpivirine, doravirine, Calanolide A, capravirine, epivir, adefovir, dapivirine, lersivirine, alovudine, elvucitabine, TMC-278, DPC-083, amdoxovir, (−)-beta-D-2,6-diamino-purine dioxolane, MIV-210 (FLG), DFC (dexelvucitabine), dioxolane thymidine, L697639, atevirdine (U87201E), MIV-150, GSK-695634, GSK-678248, TMC-278, KP1461, KP-1212, lodenosine (FddA), 5-[(3,5-dichlorophenyl)thio]-4-isopropyl-1-(4-pyridylmethyl)imidazole-2-methanol carbamic acid, (−)-I2-D-2,6-diaminopurine dioxolane, AVX-754, BCH-13520, BMS-56190 ((4S)-6-chloro-4-[(1E)-cyclopropylethenyl]-3,-4-dihydro-4-trifluoromethyl-2(1H)-quinazolinone), TMC-120, L697639, atazanavir, darunavir, fosamprenavir, indinavir, nelfinavir, ritonavir, saquinavir, tipranavir, lopinavir, amprenavir, telinavir (SC-52151), droxinavir, emtriva, invirase, agenerase, TMC-126, mozenavir (DMP-450), JE-2147 (AG1776), L-756423, KNI-272, DPC-681, DPC-684, BMS 186318, droxinavir (SC-55389a), DMP-323, KNI-227, 1-[(2-hydroxyethoxy)methyl]-6-(phenylthio)-thymine, AG-1859, RO-033-4649, R-944, DMP-850, DMP-851, brecanavir (GW640385), or salts, solvates or combinations thereof.

Provided herein, in various aspects, are compositions useful for the prevention of herpesvirus infection in a herpesvirus seronegative subject, the compositions comprising the active agent valacyclovir, or a salt or solvate thereof, wherein the compositions further comprise and/or are administered with one or more additional active agents. Further provided herein, in various aspects, are methods of preventing herpesvirus infection in a herpesvirus seronegative subject comprising administering to the subject a combination of valacyclovir, or a salt or solvate thereof, and one or more additional active agents. In some instances, the combination of valacyclovir and the one or more additional active agents is administered together in one composition. In some instances, valacyclovir and the one or more additional active agents are co-administered in different compositions. In some instances, one or more of the active agents are administered to the herpesvirus seronegative subject to reduce the risk of acquiring herpesvirus infection prior to exposure to herpesvirus. In some instances, one or more active agents are administered to the herpesvirus seronegative subject to reduce the risk of acquiring herpesvirus infection after exposure to herpesvirus. In some instances, one or more of the active agents are administered to the herpesvirus seronegative subject prior to exposure to herpesvirus and one or more of the active agents are administered to the herpesvirus seronegative subject after exposure to herpesvirus. In some of those cases, the dose of one or more active agents administered prior to exposure to herpesvirus differs from the dose of one or more active agents administered after exposure to herpesvirus. In some embodiments, valacyclovir and/or one or more of the one or more additional active agents are administered using an oral formulation, for example, capsules, tablets, soft chews, gels, liquids, powders, bait drops, or pastes. Formulation of the present invention suitable for oral administration may be as a powder or aqueous or non-aqueous liquid, or oil-in-water emulsion or water-in-oil emulsion administered as a predetermined amount of active ingredient into daily feed or water supply. Formulation of the present invention suitable for parenteral administration may be as intravenous injection or infusion, intramuscular injection, or subcutaneous injection each containing a predetermined amount of the active ingredients as a solution or suspension in aqueous liquid.

In some embodiments, the pharmaceutical composition of the present invention may be formulated for long-acting administration.

Examples of HSV antiviral agents include, without limitation, valacyclovir, acyclovir, famciclovir, pritelivir, penciclovir, ganciclovir, valganciclovir, cidofovir, foscarnet, darunavir, glycyrrhizic acid, glutamine, FV-100, ASP2151, me-609, ASP2151, topical VDO, PEG-formulation (Devirex AG), vidarabine, cidofovir, crofelemer (SP-303T), EPB-348, CMX001, V212, NB-001, squaric acid, ionic zinc, sorivudine (ARYS-01), trifluridine, 882C87, merlin (ethanol and glycolic acid mixture), vitamin C, AIC316, versabase gel with Sarracenia purpurea, UB-621, lysine, edoxudine, brivudine, cytarabine, docosanol, tromantadine, resiquimod (R-848), imiquimod, resiquimod, tenofovir, tenofovir disoproxil fumarate, tenofovir alafenamide fumarate, C5A or salts, solvates, or combinations thereof.

In some embodiments, the methods for preventing herpesvirus infection in a herpesvirus seronegative subject comprise administering to the herpesvirus seronegative subject about or at least about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of valacyclovir, or a salt or solvate thereof. In some cases, valacyclovir, or a salt or solvate thereof, is administered in a dosage from about 0.01 mg/kg to about 500 mg/kg, or from about 1 mg/kg to about 500 mg/kg. In some cases, valacyclovir, or a salt or solvate thereof, is administered 1 time per week, 2 times per week, 3 times per week, 4 times per week, 5 times per week, 6 times per week, 1 time per day, 2 times per day, 3 times per day, or 4 times per day.

In some embodiments, the compositions useful for preventing herpesvirus infection in seronegative subjects comprising valacyclovir, or a salt or solvate thereof, further comprise emtricitabine. In some embodiments, the methods for preventing herpesvirus infection in seronegative subjects comprising administering to the subject valacyclovir, or a salt or solvate thereof, further comprise administering to the subject emtricitabine, or a salt or solvate thereof. In some embodiments, the methods comprise administering to the herpesvirus seronegative subject at least or about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine, or a salt or solvate thereof. In some cases, emtricitabine is administered 1 time per week, 2 times per week, 3 times per week, 4 times per week, 5 times per week, 6 times per week, 1 time per day, 2 times per day, 3 times per day, or 4 times per day. In some cases, the method of preventing herpesvirus infection in the herpesvirus seronegative subject comprises administering to the herpesvirus seronegative subject from about 0.01 mg/kg to about 500 mg/kg of valacyclovir, or a salt or solvate thereof, and from about 0.01 mg/kg to about 500 mg/kg of emtricitabine, or a salt or solvate thereof.

In some embodiments, the compositions useful for preventing herpesvirus infection in seronegative subjects comprising valacyclovir, or a salt or solvate thereof, further comprise lamivudine. In some embodiments, the methods for preventing herpesvirus infection in seronegative subjects comprising administering to the subject valacyclovir, or a salt or solvate thereof, further comprise administering to the subject lamivudine, or a salt or solvate thereof. In some embodiments, the methods comprise administering to the herpesvirus seronegative subject at least or 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of lamivudine, or a salt or solvate thereof. In some cases, lamivudine is administered 1 time per week, 2 times per week, 3 times per week, 4 times per week, 5 times per week, 6 times per week, 1 time per day, 2 times per day, 3 times per day, or 4 times per day. In some cases, the method of preventing herpesvirus infection in the herpesvirus seronegative subject comprises administering to the herpesvirus seronegative subject from about 0.01 mg/kg to about 500 mg/kg of valacyclovir, or a salt or solvate thereof, and from about 0.01 mg/kg to about 500 mg/kg of lamivudine, or a salt or solvate thereof.

In some embodiments, the compositions useful for preventing herpesvirus infection in seronegative subjects comprising valacyclovir, or a salt or solvate thereof, further comprise glycyrrhizic acid. In some embodiments, the methods for preventing herpesvirus infection in seronegative subjects comprising administering to the subject valacyclovir, or a salt or solvate thereof, further comprise administering to the subject glycyrrhizic acid, or a salt or solvate thereof. In some embodiments, the methods comprise administering to the herpesvirus seronegative subject at least or about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of glycyrrhizic acid, or a salt or solvate thereof. In some cases, glycyrrhizic acid is administered 1 time per week, 2 times per week, 3 times per week, 4 times per week, 5 times per week, 6 times per week, 1 time per day, 2 times per day, 3 times per day, or 4 times per day. In some cases, the method of preventing herpesvirus infection in the herpesvirus seronegative subject comprises administering to the herpesvirus seronegative subject from about 0.01 mg/kg to about 500 mg/kg of valacyclovir, or a salt or solvate thereof, and from about 0.01 mg/kg to about 500 mg/kg of glycyrrhizic acid, or a salt or solvate thereof.

In some embodiments, the compositions useful for preventing herpesvirus infection in seronegative subjects comprising valacyclovir, or a salt or solvate thereof, further comprise Sambucus nigra. In some embodiments, the methods for preventing herpesvirus infection in seronegative subjects comprising administering to the subject valacyclovir, or a salt or solvate thereof, further comprise administering to the subject Sambucus nigra, or a salt or solvate thereof. In some embodiments, the methods comprise administering to the herpesvirus seronegative subject at least or about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of Sambucus nigra, or a salt or solvate thereof. In some cases, Sambucus nigra is administered 1 time per week, 2 times per week, 3 times per week, 4 times per week, 5 times per week, 6 times per week, 1 time per day, 2 times per day, 3 times per day, or 4 times per day. In some cases, the method of preventing herpesvirus infection in the herpesvirus seronegative subject comprises administering to the herpesvirus seronegative subject from about 0.01 mg/kg to about 500 mg/kg of valacyclovir, or a salt or solvate thereof, and from about 0.01 mg/kg to about 500 mg/kg of Sambucus nigra, or a salt or solvate thereof.

In some embodiments, the compositions useful for preventing herpesvirus infection in seronegative subjects comprising valacyclovir, or a salt or solvate thereof, further comprise pritelivir. In some embodiments, the methods for preventing herpesvirus infection in seronegative subjects comprising administering to the subject valacyclovir, or a salt or solvate thereof, further comprise administering to the subject pritelivir, or a salt or solvate thereof. In some embodiments, the methods comprise administering to the herpesvirus seronegative subject at least 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of pritelivir, or a salt or solvate thereof. In some cases, pritelivir is administered 1 time per week, 2 times per week, 3 times per week, 4 times per week, 5 times per week, 6 times per week, 1 time per day, 2 times per day, 3 times per day, or 4 times per day. In some cases, the method of preventing herpesvirus infection in the herpesvirus seronegative subject comprises administering to the herpesvirus seronegative subject from about 0.01 mg/kg to about 500 mg/kg of valacyclovir, or a salt or solvate thereof, and from about 0.01 mg/kg to about 500 mg/kg of pritelivir, or a salt or solvate thereof.

In some embodiments, the compositions useful for preventing herpesvirus infection in seronegative subjects comprising valacyclovir, or a salt or solvate thereof, further comprise pritelivir and emtricitabine. In some embodiments, the methods for preventing herpesvirus infection in seronegative subjects comprising administering to the subject valacyclovir, or a salt or solvate thereof, further comprise administering to the subject pritelivir, or a salt or solvate thereof and emtricitabine, or a salt or solvate thereof. In some embodiments, the methods comprise administering to the herpesvirus seronegative subject at least or about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of pritelivir, or a salt or solvate thereof. In some embodiments, the methods comprise administering to the herpesvirus seronegative subject at least or about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine, or a salt or solvate thereof. In some cases, pritelivir is administered 1 time per week, 2 times per week, 3 times per week, 4 times per week, 5 times per week, 6 times per week, 1 time per day, 2 times per day, 3 times per day, or 4 times per day. In some cases, the method of preventing herpesvirus infection in the herpesvirus seronegative subject comprises administering to the herpesvirus seronegative subject from about 0.01 mg/kg to about 500 mg/kg of valacyclovir, or a salt or solvate thereof, from about 0.01 mg/kg to about 500 mg/kg of pritelivir, or a salt or solvate thereof, and from about 0.01 mg/kg to about 500 mg/kg of emtricitabine, or a salt or solvate thereof.

In some embodiments, the compositions useful for preventing herpesvirus infection in seronegative subjects comprising valacyclovir, or a salt or solvate thereof, further comprise pritelivir and lamivudine. In some embodiments, the methods for preventing herpesvirus infection in seronegative subjects comprising administering to the subject valacyclovir, or a salt or solvate thereof, further comprise administering to the subject pritelivir, or a salt or solvate thereof and lamivudine, or a salt or solvate thereof. In some embodiments, the methods comprise administering to the herpesvirus seronegative subject at least or about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of pritelivir, or a salt or solvate thereof. In some embodiments, the methods comprise administering to the herpesvirus seronegative subject at least or about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of lamivudine, or a salt or solvate thereof. In some cases, pritelivir is administered 1 time per week, 2 times per week, 3 times per week, 4 times per week, 5 times per week, 6 times per week, 1 time per day, 2 times per day, 3 times per day, or 4 times per day. In some cases, lamivudine is administered 1 time per week, 2 times per week, 3 times per week, 4 times per week, 5 times per week, 6 times per week, 1 time per day, 2 times per day, 3 times per day, or 4 times per day. In some cases, the method of preventing herpesvirus infection in the herpesvirus seronegative subject comprises administering to the herpesvirus seronegative subject from about 0.01 mg/kg to about 500 mg/kg of valacyclovir, or a salt or solvate thereof, from about 0.01 mg/kg to about 500 mg/kg of pritelivir, or a salt or solvate thereof, and from about 0.01 mg/kg to about 500 mg/kg of lamivudine, or a salt or solvate thereof.

In some embodiments, provided herein are methods of preventing herpesvirus infection/recurrence in a subject comprising administering to the subject from about 0.01 mg/kg to about 500 mg/kg of valacyclovir, or a salt or solvate thereof, once per day. In some cases, valacyclovir is administered prior to exposure to herpesvirus. In some cases, valacyclovir is administered after exposure to herpesvirus. In some cases, valacyclovir is administered both prior to and after exposure to herpesvirus. In some cases, the methods comprise administering to the subject about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of valacyclovir, or a salt or solvate thereof, once per day. In some cases, the methods comprise administering to the subject from about, 0.01 mg/kg to about 500 mg/kg, from about 0.1 mg/kg to 250 mg/kg, from about 1 mg/kg to 100 mg/kg, from about 5 mg/kg to 50 mg/kg valacyclovir, or a salt or solvate thereof, once per day.

In some embodiments, the additional antiviral agents include tenofovir, emtricitabine, lamivudine, efavirenz, raltegravir, dolutegravir, maravoric, rilpirivine, Atripla®, and salts, solvates and/or combinations thereof. In some embodiments, one of the one or more additional antiviral agents is tenofovir and the methods further comprise administering to the subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day.

In some embodiments, one of the one or more additional antiviral agents is emtricitabine and the methods further comprise administering to the subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day.

In some embodiments, one of the one of more additional antiviral agents is lamivudine and the methods further comprise administering to the subject from about 0.01 mg/kg to about 500 mg/kg of lamivudine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of lamivudine per day.

In some embodiments, one of the one of more additional antiviral agents is efavirenz and the methods further comprise administering to the subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day.

In some embodiments, one of the one of more additional antiviral agents is raltegravir and the methods further comprise administering to the subject from about 0.01 mg/kg to about 500 mg/kg of raltegravir twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of raltegravir twice per day.

In some embodiments, one of the one of more additional antiviral agents is dolutegravir and the methods further comprise administering to the subject from about 0.01 mg/kg to about 500 mg/kg of dolutegravir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of dolutegravir per day.

In some embodiments, one of the one of more additional antiviral agents is maravoric and the methods further comprise administering to the subject from about 0.01 mg/kg to about 500 mg/kg of maravoric twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of maravoric twice per day.

In some embodiments, one of the one of more additional antiviral agents is rilpirivine and the methods further comprise administering to the subject from about 0.01 mg/kg to about 500 mg/kg of rilpirivine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of rilpirivine per day.

In some embodiments, a) one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day; b) one of the one of more additional antiviral agents is efavirenz and the methods further comprise administering to the subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day; and c) one of the one or more additional antiviral agents is tenofovir and the methods further comprise administering to the subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some cases, the one or more additional antiviral agents are administered prior to, after, or both prior to and after the herpesvirus seronegative subject is exposed to herpesvirus.

In some embodiments, provided herein are methods of preventing herpesvirus infection in a herpesvirus seronegative subject comprising administering to the subject from about 0.01 mg/kg to about 500 mg/kg of acyclovir, or a salt or solvate thereof, twice per day. In some cases, acyclovir is administered prior to exposure to herpesvirus. In some cases, acyclovir is administered after exposure to herpesvirus. In some cases, acyclovir is administered both prior to and after exposure to herpesvirus. In some cases, acyclovir is administered with one or more additional active agents, e.g., one or more additional antiviral agents. In some cases, the methods comprise administering to the herpesvirus seronegative subject about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of acyclovir, or a salt or solvate thereof, twice per day. In some cases, the methods comprise administering to the herpesvirus seronegative subject from about 0.01 mg/kg to about 500 mg/kg of acyclovir, or a salt or solvate thereof, twice per day. In some embodiments, the additional antiviral agents include tenofovir, emtricitabine, lamivudine, efavirenz, raltegravir, dolutegravir, maravoric, rilpirivine, Atripla®, and salts, solvates and/or combinations thereof. In some embodiments, one of the one or more additional antiviral agents is tenofovir and the methods further comprise administering to the herpesvirus seronegative subject from about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some embodiments, one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day. In some embodiments, one of the one of more additional antiviral agents is lamivudine and the methods further comprise administering to the herpesvirus seronegative subject from about 0.01 mg/kg to about 500 mg of lamivudine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of lamivudine per day. In some embodiments, one of the one of more additional antiviral agents is efavirenz and the methods further comprise administering to the herpesvirus seronegative subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day. In some embodiments, one of the one of more additional antiviral agents is raltegravir and the methods further comprise administering to the herpesvirus seronegative subject from about 0.01 mg/kg to about 500 mg/kg of raltegravir twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of raltegravir twice per day. In some embodiments, one of the one of more additional antiviral agents is dolutegravir and the methods further comprise administering to the herpesvirus seronegative subject from about 0.01 mg/kg to about 500 mg/kg of dolutegravir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of dolutegravir per day. In some embodiments, one of the one of more additional antiviral agents is maravoric and the methods further comprise administering to the herpesvirus seronegative subject from about 0.01 mg/kg to about 500 mg/kg of maravoric twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of maravoric twice per day. In some embodiments, one of the one of more additional antiviral agents is rilpirivine and the methods further comprise administering to the herpesvirus seronegative subject from about 0.01 mg/kg to about 500 mg/kg of rilpirivine once per day, e.g., about 10.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of rilpirivine per day. In some embodiments, a) one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day; b) one of the one of more additional antiviral agents is efavirenz and the methods further comprise administering to the herpesvirus seronegative subject from about 0.01 mg/kg to about 500 mg/kg of efavirenz once per day, e.g., 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day; and c) one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some cases, the one or more additional antiviral agents are administered prior to, after, or both prior to and after the herpesvirus seronegative subject is exposed to herpesvirus.

In some embodiments, provided herein are methods of preventing herpesvirus infection in a herpesvirus seronegative subject comprising administering to the subject from about 0.01 mg/kg to about 500 mg/kg of pritelivir, or a salt or solvate thereof, once per day. In some cases, pritelivir is administered prior to exposure to herpesvirus. In some cases, pritelivir is administered after exposure to herpesvirus. In some cases, pritelivir is administered both prior to and after exposure to herpesvirus. In some cases, pritelivir is administered with one or more additional active agents, e.g., one or more additional antiviral agents. In some cases, the methods comprise administering to the herpesvirus seronegative subject about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of pritelivir, or a salt or solvate thereof, once per day. In some cases, the methods comprise administering to the herpesvirus seronegative subject from about 0.01 mg/kg to about 500 mg/kg of pritelivir, or a salt or solvate thereof, once per day. In some embodiments, the additional antiviral agents include tenofovir, emtricitabine, lamivudine, efavirenz, raltegravir, dolutegravir, maravoric, rilpirivine, Atripla®, and salts, solvates and/or combinations thereof. In some embodiments, one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some embodiments, one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day. In some embodiments, one of the one of more additional antiviral agents is lamivudine and the methods further comprise administering to the herpesvirus seronegative subject from about 0.01 mg/kg to about 500 mg/kg of lamivudine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of lamivudine per day. In some embodiments, one of the one of more additional antiviral agents is efavirenz and the methods further comprise administering to the herpesvirus seronegative subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day. In some embodiments, one of the one of more additional antiviral agents is raltegravir and the methods further comprise administering to the herpesvirus seronegative subject from about 0.01 mg/kg to about 500 mg/kg of raltegravir twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of raltegravir twice per day. In some embodiments, one of the one of more additional antiviral agents is dolutegravir and the methods further comprise administering to the herpesvirus seronegative subject from about 0.01 mg/kg to about 500 mg/kg of dolutegravir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of dolutegravir per day. In some embodiments, one of the one of more additional antiviral agents is maravoric and the methods further comprise administering to the herpesvirus seronegative subject from about 0.01 mg/kg to about 500 mg/kg of maravoric twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of maravoric twice per day. In some embodiments, one of the one of more additional antiviral agents is rilpirivine and the methods further comprise administering to the herpesvirus seronegative subject from about 0.01 mg/kg to about 500 mg/kg of rilpirivine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of rilpirivine per day. In some embodiments, a) one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day; b) one of the one of more additional antiviral agents is efavirenz and the methods further comprise administering to the herpesvirus seronegative subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day; and c) one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some cases, the one or more additional antiviral agents are administered prior to, after, or both prior to and after the herpesvirus seronegative subject is exposed to herpesvirus.

In some embodiments, provided herein are methods of preventing herpesvirus infection in a herpesvirus seronegative subject comprising administering to the subject from about 0.01 mg/kg to about 500 mg/kg of famciclovir, or a salt or solvate thereof, twice per day. In some cases, famciclovir is administered prior to exposure to herpesvirus. In some cases, famciclovir is administered after exposure to herpesvirus. In some cases, famciclovir is administered both prior to and after exposure to herpesvirus. In some cases, famciclovir is administered with one or more additional active agents, e.g., one or more additional antiviral agents. In some cases, the methods comprise administering to the herpesvirus seronegative subject about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of famciclovir, or a salt or solvate thereof, twice per day. In some cases, the methods comprise administering to the herpesvirus seronegative subject from about 0.01 mg/kg to about 500 mg/kg of famciclovir, or a salt or solvate thereof, twice per day. In some embodiments, the additional antiviral agents include tenofovir, emtricitabine, lamivudine, efavirenz, raltegravir, dolutegravir, maravoric, rilpirivine, Atripla®, and salts, solvates and/or combinations thereof. In some embodiments, one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some embodiments, one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day. In some embodiments, one of the one of more additional antiviral agents is lamivudine and the methods further comprise administering to the herpesvirus seronegative subject from about 0.01 mg/kg to about 500 mg/kg of lamivudine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of lamivudine per day. In some embodiments, one of the one of more additional antiviral agents is efavirenz and the methods further comprise administering to the herpesvirus seronegative subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day. In some embodiments, one of the one of more additional antiviral agents is raltegravir and the methods further comprise administering to the herpesvirus seronegative subject from about 0.01 mg/kg to about 500 mg/kg of raltegravir twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of raltegravir twice per day. In some embodiments, one of the one of more additional antiviral agents is dolutegravir and the methods further comprise administering to the herpesvirus seronegative subject from about 0.01 mg/kg to about 500 mg/kg of dolutegravir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of dolutegravir per day. In some embodiments, one of the one of more additional antiviral agents is maravoric and the methods further comprise administering to the herpesvirus seronegative subject from about 0.01 mg/kg to about 500 mg of maravoric twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of maravoric twice per day. In some embodiments, one of the one of more additional antiviral agents is rilpirivine and the methods further comprise administering to the herpesvirus seronegative subject from about 0.01 mg/kg to about 500 mg/kg of rilpirivine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of rilpirivine per day. In some embodiments, a) one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day; b) one of the one of more additional antiviral agents is efavirenz and the methods further comprise administering to the herpesvirus seronegative subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day; and c) one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some cases, the one or more additional antiviral agents are administered prior to, after, or both prior to and after the herpesvirus seronegative subject is exposed to herpesvirus.

In some embodiments, provided herein are methods of preventing herpesvirus infection in a herpesvirus seronegative subject comprising administering to the subject from about 0.01 mg/kg to about 500 mg/kg of tenofovir, or a salt or solvate thereof, once per day. In some cases, tenofovir is administered prior to exposure to herpesvirus. In some cases, tenofovir is administered after exposure to herpesvirus. In some cases, tenofovir is administered both prior to and after exposure to herpesvirus. In some cases, tenofovir is administered with one or more additional active agents, e.g., one or more additional antiviral agents. In some cases, the methods comprise administering to the herpesvirus seronegative subject about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir, or a salt or solvate thereof, once per day. In some cases, the methods comprise administering to the herpesvirus seronegative subject from about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir, or a salt or solvate thereof, once per day. In some embodiments, the additional antiviral agents include tenofovir, emtricitabine, lamivudine, efavirenz, raltegravir, dolutegravir, maravoric, rilpirivine, Atripla®, and salts, solvates and/or combinations thereof. In some embodiments, one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some embodiments, one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day. In some embodiments, one of the one of more additional antiviral agents is lamivudine and the methods further comprise administering to the herpesvirus seronegative subject from about 0.01 mg/kg to about 500 mg/kg of lamivudine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of lamivudine per day. In some embodiments, one of the one of more additional antiviral agents is efavirenz and the methods further comprise administering to the herpesvirus seronegative subject from about 0.01 mg/kg to about 500 mg/kg of efavirenz once per day, e.g., 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day. In some embodiments, one of the one of more additional antiviral agents is raltegravir and the methods further comprise administering to the herpesvirus seronegative subject from about 0.01 mg/kg to about 500 mg/kg of raltegravir twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of raltegravir twice per day. In some embodiments, one of the one of more additional antiviral agents is dolutegravir and the methods further comprise administering to the herpesvirus seronegative subject from about 0.01 mg/kg to about 500 mg/kg of dolutegravir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of dolutegravir per day. In some embodiments, one of the one of more additional antiviral agents is maravoric and the methods further comprise administering to the herpesvirus seronegative subject from about 0.01 mg/kg to about 500 mg/kg of maravoric twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of maravoric twice per day. In some embodiments, one of the one of more additional antiviral agents is rilpirivine and the methods further comprise administering to the herpesvirus seronegative subject from about 0.01 mg/kg to about 500 mg/kg of rilpirivine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of rilpirivine per day. In some embodiments, a) one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day; b) one of the one of more additional antiviral agents is efavirenz and the methods further comprise administering to the herpesvirus seronegative subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day; and c) one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg g of tenofovir per day. In some cases, the one or more additional antiviral agents are administered prior to, after, or both prior to and after the herpesvirus seronegative subject is exposed to herpesvirus.

In some embodiments, provided herein are methods of preventing herpesvirus infection in a herpesvirus seronegative subject comprising administering to the subject from about 0.01 mg/kg to about 500 mg/kg of ganciclovir, or a salt or solvate thereof, three times per day. In some cases, ganciclovir is administered prior to exposure to herpesvirus. In some cases, ganciclovir is administered after exposure to herpesvirus. In some cases, ganciclovir is administered both prior to and after exposure to herpesvirus. In some cases, ganciclovir is administered with one or more additional active agents, e.g., one or more additional antiviral agents. In some cases, the methods comprise administering to the herpesvirus seronegative subject about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of ganciclovir, or a salt or solvate thereof, three times per day. In some cases, the methods comprise administering to the herpesvirus seronegative subject from about 0.01 mg/kg to about 500 mg/kg of ganciclovir, or a salt or solvate thereof, three times per day. In some embodiments, the additional antiviral agents include tenofovir, emtricitabine, lamivudine, efavirenz, raltegravir, dolutegravir, maravoric, rilpirivine, Atripla®, and salts, solvates and/or combinations thereof. In some embodiments, one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some embodiments, one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day. In some embodiments, one of the one of more additional antiviral agents is lamivudine and the methods further comprise administering to the herpesvirus seronegative subject from about 0.01 mg/kg to about 500 mg of lamivudine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of lamivudine per day. In some embodiments, one of the one of more additional antiviral agents is efavirenz and the methods further comprise administering to the herpesvirus seronegative subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day. In some embodiments, one of the one of more additional antiviral agents is raltegravir and the methods further comprise administering to the herpesvirus seronegative subject from about 0.01 mg/kg to about 500 mg/kg of raltegravir twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of raltegravir twice per day. In some embodiments, one of the one of more additional antiviral agents is dolutegravir and the methods further comprise administering to the herpesvirus seronegative subject from about 0.01 mg/kg to about 500 mg/kg of dolutegravir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of dolutegravir per day. In some embodiments, one of the one of more additional antiviral agents is maravoric and the methods further comprise administering to the herpesvirus seronegative subject from about 0.01 mg/kg to about 500 mg/kg of maravoric twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of maravoric twice per day. In some embodiments, one of the one of more additional antiviral agents is rilpirivine and the methods further comprise administering to the herpesvirus seronegative subject from about 0.01 mg/kg to about 500 mg/kg of rilpirivine once per day, e.g., about 10.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of rilpirivine per day. In some embodiments, a) one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day; b) one of the one of more additional antiviral agents is efavirenz and the methods further comprise administering to the herpesvirus seronegative subject from about 0.01 mg/kg to about 500 mg/kg of efavirenz once per day, e.g., 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day; and c) one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some cases, the one or more additional antiviral agents are administered prior to, after, or both prior to and after the herpesvirus seronegative subject is exposed to herpesvirus.

In some embodiments, provided herein are methods of preventing herpesvirus infection in a herpesvirus seronegative subject comprising administering to the subject from about 0.01 mg/kg to about 500 mg/kg of glycyrrhizic acid, or a salt or solvate thereof, once per day. In some cases, glycyrrhizic acid is administered prior to exposure to herpesvirus. In some cases, glycyrrhizic acid is administered after exposure to herpesvirus. In some cases, glycyrrhizic acid is administered both prior to and after exposure to herpesvirus. In some cases, glycyrrhizic acid is administered with one or more additional active agents, e.g., one or more additional antiviral agents. In some cases, the methods comprise administering to the herpesvirus seronegative subject about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of glycyrrhizic acid, or a salt or solvate thereof, once per day. In some cases, the methods comprise administering to the herpesvirus seronegative subject from about 0.01 mg/kg to about 500 mg/kg of glycyrrhizic acid, or a salt or solvate thereof, once per day. In some embodiments, the additional antiviral agents include tenofovir, emtricitabine, lamivudine, efavirenz, raltegravir, dolutegravir, maravoric, rilpirivine, Atripla®, and salts, solvates and/or combinations thereof. In some embodiments, one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some embodiments, one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day. In some embodiments, one of the one of more additional antiviral agents is lamivudine and the methods further comprise administering to the herpesvirus seronegative subject from about 0.01 mg/kg to about 500 mg/kg of lamivudine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of lamivudine per day. In some embodiments, one of the one of more additional antiviral agents is efavirenz and the methods further comprise administering to the herpesvirus seronegative subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day. In some embodiments, one of the one of more additional antiviral agents is raltegravir and the methods further comprise administering to the herpesvirus seronegative subject from about 0.01 mg/kg to about 500 mg/kg of raltegravir twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of raltegravir twice per day. In some embodiments, one of the one of more additional antiviral agents is dolutegravir and the methods further comprise administering to the herpesvirus seronegative subject from about 0.01 mg/kg to about 500 mg/kg of dolutegravir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of dolutegravir per day. In some embodiments, one of the one of more additional antiviral agents is maravoric and the methods further comprise administering to the herpesvirus seronegative subject from about 0.01 mg/kg to about 500 mg/kg of maravoric twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of maravoric twice per day. In some embodiments, one of the one of more additional antiviral agents is rilpirivine and the methods further comprise administering to the herpesvirus seronegative subject from about 0.01 mg/kg to about 500 mg/kg of rilpirivine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of rilpirivine per day. In some embodiments, a) one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day; b) one of the one of more additional antiviral agents is efavirenz and the methods further comprise administering to the herpesvirus seronegative subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day; and c) one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some cases, the one or more additional antiviral agents are administered prior to, after, or both prior to and after the herpesvirus seronegative subject is exposed to herpesvirus.

In some embodiments, provided herein are methods of preventing herpesvirus infection in a herpesvirus seronegative subject comprising administering to the subject from about 0.01 mg/kg to about 500 mg/kg of Sambucus nigra, or a salt or solvate thereof, once per day. In some cases, Sambucus nigra is administered prior to exposure to herpesvirus. In some cases, Sambucus nigra is administered after exposure to herpesvirus. In some cases, Sambucus nigra is administered both prior to and after exposure to herpesvirus. In some cases, Sambucus nigra is administered with one or more additional active agents, e.g., one or more additional antiviral agents. In some cases, the methods comprise administering to the herpesvirus seronegative subject about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of Sambucus nigra, or a salt or solvate thereof, once per day. In some cases, the methods comprise administering to the herpesvirus seronegative subject from about 0.01 mg/kg to about 500 mg/kg of Sambucus nigra, or a salt or solvate thereof, once per day. In some embodiments, the additional antiviral agents include tenofovir, emtricitabine, lamivudine, efavirenz, raltegravir, dolutegravir, maravoric, rilpirivine, Atripla®, and salts, solvates and/or combinations thereof. In some embodiments, one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg mg of tenofovir per day. In some embodiments, one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day. In some embodiments, one of the one of more additional antiviral agents is lamivudine and the methods further comprise administering to the herpesvirus seronegative subject from about 0.01 mg/kg to about 500 mg/kg of lamivudine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of lamivudine per day. In some embodiments, one of the one of more additional antiviral agents is efavirenz and the methods further comprise administering to the herpesvirus seronegative subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day. In some embodiments, one of the one of more additional antiviral agents is raltegravir and the methods further comprise administering to the herpesvirus seronegative subject from about 0.01 mg/kg to about 500 mg/kg of raltegravir twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of raltegravir twice per day. In some embodiments, one of the one of more additional antiviral agents is dolutegravir and the methods further comprise administering to the herpesvirus seronegative subject from about 0.01 mg/kg to about 500 mg/kg of dolutegravir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of dolutegravir per day. In some embodiments, one of the one of more additional antiviral agents is maravoric and the methods further comprise administering to the herpesvirus seronegative subject from about 0.01 mg/kg to about 500 mg/kg of maravoric twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of maravoric twice per day. In some embodiments, one of the one of more additional antiviral agents is rilpirivine and the methods further comprise administering to the herpesvirus seronegative subject from about 0.01 mg/kg to about 500 mg/kg of rilpirivine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of rilpirivine per day. In some embodiments, a) one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day; b) one of the one of more additional antiviral agents is efavirenz and the methods further comprise administering to the herpesvirus seronegative subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day; and c) one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some cases, the one or more additional antiviral agents are administered prior to, after, or both prior to and after the herpesvirus seronegative subject is exposed to herpesvirus.

In some embodiments, provided herein are methods of preventing herpesvirus infection in a herpesvirus seronegative subject comprising administering to the subject from about 0.01 mg/kg to about 500 mg/kg of valganciclovir, or a salt or solvate thereof, once per day. In some cases, valganciclovir is administered prior to exposure to herpesvirus. In some cases, valganciclovir is administered after exposure to herpesvirus. In some cases, valganciclovir is administered both prior to and after exposure to herpesvirus. In some cases, valganciclovir is administered with one or more additional active agents, e.g., one or more additional antiviral agents. In some cases, the methods comprise administering to the herpesvirus seronegative subject about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of valganciclovir, or a salt or solvate thereof, once per day. In some cases, the methods comprise administering to the herpesvirus seronegative subject from about 0.01 mg/kg to about 500 mg/kg of valganciclovir, or a salt or solvate thereof, once per day. In some embodiments, the additional antiviral agents include tenofovir, emtricitabine, lamivudine, efavirenz, raltegravir, dolutegravir, maravoric, rilpirivine, Atripla®, and salts, solvates and/or combinations thereof. In some embodiments, one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some embodiments, one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day. In some embodiments, one of the one of more additional antiviral agents is lamivudine and the methods further comprise administering to the herpesvirus seronegative subject from about 0.01 mg/kg to about 500 mg/kg of lamivudine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of lamivudine per day. In some embodiments, one of the one of more additional antiviral agents is efavirenz and the methods further comprise administering to the herpesvirus seronegative subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day. In some embodiments, one of the one of more additional antiviral agents is raltegravir and the methods further comprise administering to the herpesvirus seronegative subject from about 0.01 mg/kg to about 500 mg/kg of raltegravir twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of raltegravir twice per day. In some embodiments, one of the one of more additional antiviral agents is dolutegravir and the methods further comprise administering to the herpesvirus seronegative subject from about 0.01 mg/kg to about 500 mg/kg of dolutegravir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of dolutegravir per day. In some embodiments, one of the one of more additional antiviral agents is maravoric and the methods further comprise administering to the herpesvirus seronegative subject from about 0.01 mg/kg to about 500 mg/kg of maravoric twice per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of maravoric twice per day. In some embodiments, one of the one of more additional antiviral agents is rilpirivine and the methods further comprise administering to the herpesvirus seronegative subject from about 0.01 mg/kg to about 500 mg/kg of rilpirivine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of rilpirivine per day. In some embodiments, a) one of the one or more additional antiviral agents is emtricitabine and the methods further comprise administering to the herpesvirus seronegative subject from about 0.01 mg/kg to about 500 mg/kg of emtricitabine once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of emtricitabine per day; b) one of the one of more additional antiviral agents is efavirenz and the methods further comprise administering to the herpesvirus seronegative subject from about 0.01 mg/kg to about 500 mg/kg of efavirenz once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of efavirenz per day; and c) one of the one or more additional antiviral agents is tenofovir and the methods further comprise administering to the herpesvirus seronegative subject from about 0.01 mg/kg to about 500 mg/kg of tenofovir once per day, e.g., about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg of tenofovir per day. In some cases, the one or more additional antiviral agents are administered prior to, after, or both prior to and after the herpesvirus seronegative subject is exposed to herpesvirus.

In some aspects, described herein are compositions for administration to a herpesvirus

seronegative subject, the compositions comprising a first dose of a first antiviral agent; wherein the composition is administered to the subject after exposure to herpesvirus. In some embodiments, the composition is a post-exposure composition. In some embodiments, the first antiviral agent is an HSV antiviral agent. In some embodiments, the first antiviral agent comprises valacyclovir, acyclovir, famciclovir, pritelivir, penciclovir, ganciclovir, valganciclovir, cidofovir, foscarnet, darunavir, glycyrrhizic acid, glutamine, FV-100, ASP2151, me-609, ASP2151, topical VDO, PEG-formulation (Devirex AG), vidarabine, cidofovir, crofelemer (SP-303T), EPB-348, CMX001, V212, NB-001, squaric acid, ionic zinc, sorivudine (ARYS-01), trifluridine, 882C87, merlin (ethanol and glycolic acid mixture), vitamin C, AIC316, versabase gel with Sarracenia purpurea, UB-621, lysine, edoxudine, brivudine, cytarabine, docosanol, tromantadine, resiquimod (R-848), imiquimod, resiquimod, tenofovir, tenofovir disoproxil fumarate, tenofovir alafenamide fumarate, amenamevir, N-methancocarbathymidine (N-MCT), or salts, solvates, or combinations thereof. In some embodiments, the composition and/or first antiviral agent is useful for the prevention of herpesvirus infection in the seronegative subject when the composition and/or first antiviral agent is administered to the subject after exposure to herpesvirus. In some cases, the composition and/or first viral agent is useful for the prevention of herpesvirus infection in the seronegative subject when the composition and/or first viral agent is administered to the subject prior to and after exposure to herpesvirus. In some embodiments, the composition and/or antiviral agent is useful to suppress herpesvirus replication in the subject when the composition and/or antiviral agent is administered to the subject after exposure to herpesvirus. In some cases, the composition and/or antiviral agent is useful to suppress herpesvirus replication in the subject when the composition and/or antiviral agent is administered to the subject prior to and after exposure to herpesvirus. In some embodiments, the composition and/or antiviral agent is useful to suppress herpesvirus activation in the subject when the composition and/or antiviral agent is administered to the subject after exposure to herpesvirus. In some cases, the composition and/or antiviral agent is useful to suppress herpesvirus activation in the subject when the composition is administered to the subject prior to and after exposure to herpesvirus. In some cases, the composition and/or antiviral agent is useful to suppress herpesvirus re-activation in the subject when the composition is administered to a seropositive subject. In some cases, the composition and/or antiviral agent is useful to treat herpesvirus infection in the subject when the composition is administered to a seropositive subject.

In some embodiments, the composition is useful to reduce the risk of herpesvirus infection in the subject when the composition is administered to the subject after exposure to herpesvirus. In some embodiments, the risk of herpesvirus infection is reduced by at least about 10%, 20%, 30%, 40%, 50%, 60%, 70%, 80%, 90%, or 100%, as compared to the risk of herpesvirus infection without administration of the composition.

In some embodiments, a very high dose of the first antiviral agent is from about 500 mg/kg to about 1000 mg/kg. In some cases, the very high dose of the antiviral agent is between about 500 mg/kg and about 1000 mg/kg, between about 600 mg/kg and about 1000 mg/kg, between about 700 mg/kg and about 1000 mg/kg, between about 800 mg/kg and about 1000 mg/kg, between about 900 mg/kg and about 1000 mg/kg. In some embodiments, the very high dose of the first antiviral agent is an amount greater than an amount of the first antiviral agent useful for treatment of an outbreak in a herpesvirus seropositive subject. In some embodiments, the very high dose of the first antiviral agent is configured for administration to the subject within 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 36 hours, 2 days, 60 hours, 3 days, 4 days, 5 days, 6 days, 1 week, 2 weeks, 1 month, or 2 months of herpesvirus exposure. In some embodiments, the very high dose is administered once a month, once every 3 weeks, once every 2 weeks, once every week, 6 times a week, 5 times a week, 4 times a week, 3 times a week, 2 times a week, 3 times per day, 2 times per day or once per day.

In some embodiments, the composition further comprises a second, third, fourth, or fifth antiviral agent. In alternative or additional embodiments, the composition is configured for administration with a second, third, fourth, and/or fifth antiviral agent. In some embodiments, the second, third, fourth, and/or fifth antiviral agent is present in the composition at a second, third, fourth, and/or fifth very high dose. In some cases, the second, third, fourth, and/or fifth antiviral agent comprises the same or different active agent as the first antiviral agent. In some embodiments, the second, third, fourth, and/or fifth antiviral agent comprises valacyclovir, acyclovir, famciclovir, pritelivir, penciclovir, ganciclovir, valganciclovir, cidofovir, foscarnet, darunavir, glycyrrhizic acid, glutamine, FV-100, ASP2151, me-609, ASP2151, topical VDO, PEG-formulation (Devirex AG), vidarabine, cidofovir, crofelemer (SP-303T), EPB-348, CMX001, V212, NB-001, squaric acid, ionic zinc, sorivudine (ARYS-01), trifluridine, 882C87, merlin (ethanol and glycolic acid mixture), vitamin C, AIC316, versabase gel with Sarracenia purpurea, UB-621, lysine, edoxudine, brivudine, cytarabine, docosanol, tromantadine, resiquimod (R-848), imiquimod, resiquimod, tenofovir, tenofovir disoproxil fumarate, tenofovir alafenamide fumarate, include GSK208141 (gD2t, GSK glycoprotein D (gD)-Alum/3-deacylated form of monophosphoryl lipid A), Herpes Zoster GSK 1437173A, gD2-AS04, Havrix™, gD-Alum, Zostavax/Zoster vaccine (V211, V212, V210), HSV529, HerpV (AG-707 rh-Hsc70 polyvalent peptide complex), VCL-HB01, VCL-HMO1, pPJV7630, GEN-003, SPL7013 gel (VivaGel™), GSK324332A, GSK1492903A, VariZIG™, and Varivax, maraviroc, enfuvirtide, vicriviroc, cenicriviroc, lbalizumab, fostemsavir (BMS-663068), ibalizumab (TMB-355, TNX-355), PRO 140, b12 antibody, DCM205, DARPins, caprine antibody, VIR-576, enfuvirtide (T-20), AMD11070, PR0542, SCH-C, T-1249, cyanovirin, griffithsen, lectins, pentafuside, dolutegravir, elvitegravir, raltegravir, globoidnan A, MK-2048, B1224436, cab otegravir, GSK 1265744, GSK-572, MK-0518, abacavir, didanosine, emtrictabine, lamivudine, stavudine, tenofovir, tenofovir disoporoxil fumarate, zidovudine, apricitabine, stampidine, elvucitabine, racivir, amdoxovir, stavudine, zalcitabine, festinavir, dideoxycytidine ddC, azidothymidine, tenofovir alafenamide fumarate, entecavir, delavirdine, efavirenz, etravirine (TMC-125), nevirapine, rilpivirine, doravirine, Calanolide A, capravirine, epivir, adefovir, dapivirine, lersivirine, alovudine, elvucitabine, TMC-278, DPC-083, amdoxovir, (−)-beta-D-2,6-diamino-purine dioxolane, MIV-210 (FLG), DFC (dexelvucitabine), dioxolane thymidine, L697639, atevirdine (U87201E), MIV-150, GSK-695634, GSK-678248, TMC-278, KP1461, KP-1212, lodenosine (FddA), 54(3,5-di chlorophenyl)thio]-4-isopropyl-1-(4-pyridylmethyl)imidazole-2-m ethanol carbamic acid, (−)-12-D-2,6-diaminopurine dioxolane, AVX-754, BCH-13520, BMS-56190 ((4S)-6-chloro-4-[(1E)-cyclopropylethenyl]-3,-4-dihydro-4-trifluoromethyl-2(1H)-quinazolinone), TMC-120, L697639, atazanavir, darunavir, fosamprenavir, indinavir, nelfinavir, ritonavir, saquinavir, tipranavir, lopinavir, amprenavir, telinavir (SC-52151), droxinavir, emtriva, invirase, agenerase, TMC-126, mozenavir (DMP-450), JE-2147 (AG1776), L-756423, KNI-272, DPC-681, DPC-684, BMS 186318, droxinavir (SC-55389a), DMP-323, KNI-227, 1-[(2-hydroxyethoxy)methyl]-(phenylthio)-thymine, AG-1859, RO-033-4649, R-944, DMP-850, DMP-851, brecanavir (GW640385), nonoxynol-9, sodium dodecyl sulfate, Savvy (1.0% C31G), BufferGel, carrageenans, VivaGel, PRO-2000, also known as PRO 2000/5, naphthalene 2-sulfonate polymer, or polynaphthalene sulphonate, amphotericin B, sulfamethoxazole, trimethoprim, clarithromycin, daunorubicin, fluconazole, doxorubicin, anidulafungin, immune globulin, gamma globulin, dronabinol, megestrol acetate, atovaquone, rifabutin, pentamidine, trimetrexate glucuronate, leucovorin, alitretinoin gel, erythropoietin, calcium hydroxyapatite, poly-L-lactic acid, somatropin rDNA, itraconazole, paclitaxel, voriconazole, cidofovir, fomivirsen, azithromycin, ruxolitinib, tocilizumab, bevirimat, TRIM5alpha, Tat antagonists, trichosanthin, abzyme, calanolide A, ceragenin, cyanovirin-N, diarylpyrimidines, epigallocatechin gallate (EGCG), foscarnet, griffithsin, hydroxycarbamide, miltefosine, portmanteau inhibitors, scytovirin, seliciclib, synergistic enhancers, tre recombinase, zinc finger protein transcription factor, KP-1461, BIT225, aplaviroc, atevirdine, brecanavir, capravirine, dexelvucitabine, emivirine, lersivirine, lodenosine, loviride, fomivirsen, glycyrrhizic acid (anti-inflammatory, inhibits 1 lbeta-hydroxysteroid dehydrogenase), zinc salts, cellulose sulfate, cyclodextrins, dextrin-2-sulfate, NCP7 inhibitors, AMD-3100, BMS-806, BMS-793, C31G, carrageenan, CD4-IgG2, cellulose acetate phthalate, mAb 2G12, mAb b12, Merck 167, plant lectins, poly naphthalene sulfate, poly sulfo-styrene, PRO2000, PSC-Rantes, SCH-C, SCH-D, T-20, TMC-125, UC-781, UK-427, UK-857, Carraguard (PC-515), brincidofovir (CMX001), zidovudine, virus-specific cytotoxic T cells, idoxuridine, podophyllotoxin, rifampicin, metisazone, interferon alfa 2b (Intron-A), peginterferon alfa-2a, ribavirin, moroxydine, pleconaril, BCX4430, taribavirin (viramidine, ICN 3142), favipiravir, rintatolimod, ibacitabine, (5-iodo-2′-deoxycytidine), methisazone (metisazone), ampligen, arbidol, Atripla®, combivir, imunovir, nexavir, trizivir, truvada, lamivudine, dideoxyadenosine, floxuridine, idozuridine, inosine pranobex, 2′-deoxy-5-(methylamino)uridine, digoxin, imiquimod, interferon type III, interferon type II, interferon type I, tea tree oil, glycyrrhizic acid, fialuridine, telbivudine, adefovir, etecavir, lamivudine, clevudine, asunaprevir, boceprevir, faldaprevir, grazoprevir, paritaprevir, ritonavir, telaprevir, simeprevir, sofosbuvir, ACH-3102, daclatasvir, deleobuvir, elbasvir, ledipasvir, MK-3682, MK-8408, samatasvir, ombitasvir, entecavir, elderberry sambucus, umifenovir, amantadine, rimantadine, oseltamivir, zanamivir, peramivir, laninamivir, pyrrole polyamides, lopinavir, amenamevir, N-methancocarbathymidine (N-MCT), or salts, solvates, and/or combinations thereof.

In some embodiments, the second, third, fourth, and/or fifth antiviral agent is an HIV antiviral comprising maraviroc, enfuvirtide, vicriviroc, cenicriviroc, lbalizumab, fostemsavir (BMS-663068), ibalizumab (TMB-355, TNX-355), PRO 140, b12 antibody, DCM205, DARPins, caprine antibody, VIR-576, enfuvirtide (T-20), AMD11070, PR0542, SCH-C, T-1249, cyanovirin, griffithsen, lectins, pentafuside, dolutegravir, elvitegravir, raltegravir, globoidnan A, MK-2048, BI224436, cabotegravir, GSK 1265744, GSK-572, MK-0518, abacavir, didanosine, emtrictabine, lamivudine, stavudine, tenofovir, tenofovir disoporoxil fumarate, zidovudine, apricitabine, stampidine, elvucitabine, racivir, amdoxovir, stavudine, zalcitabine, festinavir, dideoxycytidine ddC, azidothymidine, tenofovir alafenamide fumarate, entecavir, delavirdine, efavirenz, etravirine (TMC-125), nevirapine, rilpivirine, doravirine, Calanolide A, capravirine, epivir, adefovir, dapivirine, lersivirine, alovudine, elvucitabine, TMC-278, DPC-083, amdoxovir, (−)-beta-D-2,6-diamino-purine dioxolane, MIV-210 (FLG), DFC (dexelvucitabine), dioxolane thymidine, L697639, atevirdine (U87201E), MIV-150, GSK-695634, GSK-678248, TMC-278, KP1461, KP-1212, lodenosine (FddA), 5-[(3, 5-di chlorophenyl)thio]-4-isopropyl-1-(4-pyridylmethyl)imidazole-2-methanol carbamic acid, (−)-I2-D-2,6-diaminopurine dioxolane, AVX-754, BCH-13520, BMS-56190 ((4S)-6-chloro-4-[(1E)-cyclopropylethenyl]-3,-4-dihydro-4-trifluoromethyl-2(1H)-quinazolinone), TMC-120, L697639, atazanavir, darunavir, fosamprenavir, indinavir, nelfinavir, ritonavir, saquinavir, tipranavir, lopinavir, amprenavir, telinavir (SC-52151), droxinavir, emtriva, invirase, agenerase, TMC-126, mozenavir (DMP-450), JE-2147 (AG1776), L-756423, KNI-272, DPC-681, DPC-684, BMS 186318, droxinavir (SC-55389a), DMP-323, KNI-227, 1-[(2-hydroxyethoxy)methyl]-6-(phenylthio)-thymine, AG-1859, RO-033-4649, R-944, DMP-850, DMP-851, brecanavir (GW640385), or salts, solvates or combinations thereof.

In some embodiments, the second, third, fourth, and/or fifth very high dose of a second antiviral agent is from about 500 mg/kg to about 1000 mg/kg. In some cases, the very high dose of the antiviral agent is between about 500 mg/kg and about 1000 mg/kg, between about 600 mg/kg and about 1000 mg/kg, between about 700 mg/kg and about 1000 mg/kg, between about 800 mg/kg and about 1000 mg/kg, between about 900 mg/kg and about 1000 mg/kg. In some embodiments, the very high dose of the second, third, fourth, and/or fifth antiviral agent is an amount greater than an amount of the first antiviral agent useful for treatment of an outbreak in a herpesvirus seropositive subject. In some embodiments, the very high dose of the second, third, fourth, and/or fifth antiviral agent is configured for administration to the subject within 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 36 hours, 2 days, 60 hours, 3 days, 4 days, 5 days, 6 days, 1 week, 2 weeks or 1 month of herpesvirus exposure. In some embodiments, the very high dose is administered once a month, once every 3 weeks, once every 2 weeks, once every week, 6 times a week, 5 times a week, 4 times a week, 3 times a week, 2 times a week, 5 times per day, 4 times per day, 3 times per day, 2 times per day or once per day.

Provided herein, in various aspects, are compositions comprising one or more active agents, for example, antiviral agents, formulated with one or more pharmaceutically acceptable excipients, diluents, carriers and/or adjuvants. In addition, compositions of the disclosure include active agents formulated with one or more pharmaceutically acceptable auxiliary substances. Auxiliary substances, such as preservatives, stabilizers, surfactants, viscosity agents, disintegration agents, flavorings, colors and the like are readily available to the public.

In many embodiments, the active agent is formulated into a pharmaceutical composition by combination with appropriate, pharmaceutically acceptable carriers or diluents, into solid, semi-solid, or liquid forms, such as soft chews, tablets, capsules, gels, liquids, powders, bait drops, and pastes. For oral preparations, the active agent may be used alone or in combination with appropriate additives to make soft chews, tablets, capsules, gels, liquids, powders, bait drops, or pastes, for example, with conventional additives, such as flavorings, stabilizers, viscosity modifiers, surfactants/solubilizers, preservatives, filler components, and/or disintegration agents.

In some embodiments, an active agent of a composition herein is formulated into a soft chew by mixing with pH stabilizers, viscosity modifiers, surfactants/solubilizers, flavorings, filler components, and optionally preservatives or disintegration agents and processing into individual dosage units by hand or with a forming machine.

In some embodiments, an active agent of a composition if formulated into a tablet, including chewable tablets, pills, dragees, lozenges, and the like, capsules, including, soft or hard gelatin or non-gelatin capsules, caplets, bait drops and the like, or powders for oral ingestion by an animal to be treated. Such formulations can comprise pharmaceutically acceptable carriers including solid diluents or fillers, sterile aqueous media and various non-toxic organic solvents. A solid carrier can be one or more substances which can also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets can contain from about one (1) to about seventy (70) percent of the active compound. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Generally, an agent (e.g., nucleic acid sequence or polypeptide) will be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

In some embodiments, an active agent of a composition is formulated into a gel by mixing with pH stabilizers, viscosity modifiers, surfactants/solubilizers, flavorings, and optionally preservatives, and/or disintegration agents.

In some embodiments, an active ingredient is formulated into a liquid by mixing with pH stabilizers, surfactants/solubilizers, flavorings, and optionally preservatives.

In some embodiments, an active ingredient is formulated into a paste by mixing with pH stabilizers, viscosity modifiers, surfactants/solubilizers, flavorings, and optionally, preservatives, and/or disintegration agents.

pH stabilizer components are well known in the art and typically may include citric acid, acetate, malate, EDTA, ascorbic acid, BHA, and/or the like.

Viscosity modifiers are well known in the art and typically may include PEG 200, PEG 300, PEG 400, PEG 600, glycerin, polyoxamers, polyoxyethylene sorbitan, hydrogenated castor oil, triethanolamine, and/or the like.

Surfactants/solubilizers are well known in the art and typically may include sodium lauryl sulfate, glyceryl monooeleate, sorbitan esters, and/or the like.

Flavorings are well known in the art and typically may include apple-molasses (such as PC-0655, Pet Flavors LLC), carrot, molasses, sugar, watermelon, orange, peppermint, alfalfa, apple, cherry, strawberry, and/or the like.

Preservatives are well known in the art and typically may include potassium sorbate, sodium benzoate, calcium propionate, and/or the like.

Disintegration agents are well known in the art and typically may include sodium starch glycoalate, pregelatinized corn starch, crospovidone, and/or the like.

Binders are well known in the art and are used to impart cohesive qualities to a solid dosage formulation, and thus ensure that a tablet or bead or granule remains intact after the formation of the dosage forms. Suitable binder materials may include, starch, pregelatinized starch, gelatin, sugars, polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone, and/or the like.

Lubricants are well known in the art and are used to facilitate tablet manufacture and typically may include magnesium stearate, calcium stearate, stearic acid, glycerol behenate, polyethylene glycol, talc, and mineral oil, and/or the like.

If desired, the soft chews, tablets, capsules, gels, liquids, powders, bait drops, and pastes can also contain minor amount of nontoxic auxiliary substances such as wetting or emulsifying agents, or dyes.

In some embodiments, unit dosage forms for oral administration, such as soft chews, tablets, capsules, gels, liquids, powders, bait drops, and pastes, are provided wherein each dosage unit (e.g., soft chew, syringe) comprises a predetermined amount of the composition comprising one or more active agents.

In some embodiments, unit dosage forms for parenteral administration, such as intravenous injection or infusion, intramuscular injection, or subcutaneous injection each containing a predetermined amount of the composition comprising one or more active ingredients.

Some embodiments of a composition of the present invention relate to an edible delivery vehicle or soft chew for the delivery of an active agent to an organism. Such organism may be any organism. Especially considered organisms include equines and camelids.

In an embodiment, a composition of the soft chew comprises a starch component, a sugar component, and an oil component. Generally, in various embodiments, the starch component comprises about 5 percent to about 60 percent of the soft chew, the sugar component comprises about 5 percent to about 75 percent of the soft chew, and the oil component comprises about 1 percent to about 40 percent of the soft chew. The percentages of the starch component, sugar component, and/or oil component may be varied depending upon the end use and desired consistency of the soft chew.

In an alternate embodiment, the starch component comprises about 15 percent to about 40 percent of the soft chew, the sugar component comprises about 15 percent to about 60 percent of the soft chew, and the oil component comprises about 5 percent to about 30 percent of the soft chew.

In an alternate embodiment, the starch component comprises about 25 percent to about 35 percent of the soft chew, the sugar component comprises about 25 percent to about 50 percent of the soft chew, and the oil component comprises about 7 percent to about 15 percent of the soft chew.

The starch component may comprise starch from any source and may act as a binder in the soft chew. In an embodiment, the starch component is derivatized and/or pregelatinized. In a preferred embodiment, the starch component is highly derivatized. Some starches that can serve as a base starch for derivatization include regular corn, waxy corn, potato, tapioca, rice, etc. Suitable types of derivatizing agents for the starch include, but are not limited to, ethylene oxide, propylene oxide, acetic anhydride, and succinic anhydride, and other food approved esters or ethers, introducing such chemicals alone or in combination with one another.

In various embodiments, prior cross-linking of the starch in the starch component may or may not be necessary, based on the pH of the system and the temperature used to form the product. The starch component may also include amylaceous ingredients. The amylaceous ingredients can be gelatinized or cooked before or during the forming step to achieve the desired matrix characteristics. If gelatinized starch is used, it may be possible to prepare the product of the subject invention or perform the process of the subject invention without heating or cooking. However, ungelatinized (ungelled) or uncooked starch may also be used.

The sugar component may act as a sweetener and may comprise sugars including, but not limited to, white sugar, corn syrup, sorbitol (solution), multirole (syrup), oligosaccharide, isomaltooligosaccharide, fructose, lactose, glucose, lycasin, xylitol, lactitol, erythritol, mannitol, isomaltose, polydextrose, raffinose, dextrin, galactose, sucrose, invert sugar, honey, molasses, polyhydric alcohols and other similar saccharines oligomers and polymers and mixture thereof. In addition, artificial sweeteners such as saccharine, aspartame and other dipeptide sweeteners may be present and sugarless can include solid polyols such as Sorbitol, Mannitol and Xylitol. Examples of various well established sources of a portion of these sugars are, corn syrup solids, malt syrup, hydrolyzed corn starch, hydrol (syrup from glucose manufacturing operations), raw and refined cane and beet sugars, and the like.

The oil component may act as a humectant and may comprise more than one oil including, but not limited to, fat or fats, both natural and synthetic. Oil employed as an ingredient in the soft chew may be a saturated or unsaturated liquid fatty acid, its glyceride derivatives or fatty acid derivatives of plant or animal origin or a mixture thereof. A source for typical animal fats or oils are fish oil, chicken fat, tallow, choice white grease, prime steam lard and mixtures thereof. However, other animal fats are also suitable for use in the soft chew. Suitable sources for vegetable fats or oils can be derived palm oil, palm hydrogenated oil, corn germ hydrogenated oil, castor hydrogenated oil, cottonseed oil, soybean oil, olive oil, peanut oil, pahn olein oil, Cacao fat, margarine, butter, shortening and palm stearin oil, and mixtures thereof. Additionally, a mixture of animal or vegetable oils or fats is suitable for use in the matrix.

Various other embodiments further comprise a flavoring component. Such flavoring component, in an embodiment, is to improve and/or change the palatability of the soft chew. Any flavoring in the flavoring component may be used. Examples of suitable flavor for the flavoring component includes, but is not limited to, strawberry flavor, tutti fruity flavor, orange flavor, banana flavor, mint flavor, and an apple-molasses. A suitable source for an apple-molasses flavoring component is Pharma Chemie, 1877 Midland Street, P.O. Box 326, Syracuse, NE 68446-0326, under a product name of Sweet-Apple Molasses Flavoring, Product Code PC-0555.

Various embodiments further comprise a stabilizer and/or lubricating component. In an embodiment, suitable stabilizer components are Magnesium Stearate, citric acid, sodium citrate, and/or the like. However, stabilizer components are common in the art and any suitable one or mixture of more than one may be used. In an embodiment, a stabilizer component comprises about 0.0 percent to about 3.0 percent of the soft chew. In an alternate embodiment, a stabilizer component comprises about 0.5 percent to about 1.5 percent of the soft chew.

Various embodiments further comprise an emulsifier component. A suitable emulsifier component is a glycerin, glycerin fatty acid ester, sorbitan monostearate, sucrose fatty acid ester, lecithin, polyethylene glycol, mixtures thereof, and the like. However, emulsifier components are well known in the art field and any emulsifier component may be used. Generally, the amount of emulsifier component added may affect the stickiness of the soft chew. The greater the concentration of glycerin, the stickier the soft chew, hi an embodiment, an emulsifier component comprises about 0.0 percent to about 40 percent of the soft chew. In an alternate embodiment, an emulsifier component comprises about 5.0 percent to about 30 percent of the soft chew. In an alternate embodiment, an emulsifier component comprises about 10 percent to about 20 percent of the soft chew.

In various embodiments, a moisture component is in the composition. In an embodiment, a moisture component comprises about 0.0 percent to about 15 percent. In an alternate embodiment, a moisture component comprises about 2.0 percent to about 10 percent. In an alternate embodiment, a moisture component comprises about 5.0 percent to about 7.5 percent. In various embodiments, an active agent component is added to the composition.

Further, embodiments of the present invention comprise processes for forming a soft chew of the present invention. In an embodiment, a process of the present invention comprises forming the soft chew in a forming machine, such that the formed soft chew and/or punched out soft chew, not an extrudate. In an embodiment, ingredients of a soft chew of the present invention are uniformly dispersed throughout the soft chew, in another embodiment of a soft chew of the present invention; ingredients are at least partially separated, such as when an embodiment has distinct layers, segments, and/or zones.

Some embodiments of a composition of the present invention relate to an edible paste for the delivery of an active agent to an organism. In some embodiments, a composition of the paste comprises an effective amount of an active agent, fumed silica, a viscosity modifier, a carrier, and optionally, an absorbent, colorant, stabilizer, surfactant, or preservative.

The fumed silica component of the paste formulation is used as a thickening agent. Preferred pastes comprise from about 1% to about 20%, based upon total weight of solution, with from about 1% to about 6% being preferred. Amounts of about 0 02% to about 20%, about 1% to 6.5% or about 1% to about 4% or 5% are also preferred.

Especially preferred viscosity modifiers include PEG 200, PEG 300. PEG 400, and PEG 600. Other hydroxyl-containing viscosity modifiers include block copolymer mixtures of polyoxyalkylene compounds, i.e., poloxamers including ethylene oxide. Other suitable viscosity modifiers useful as of the present invention include polyoxyethylene sorbit in monoleate (Polysorbate 80); polyethylene glycols (Pluracols); nonylphenol ethoxylates (Surnics); and linear alcohol ethoxylates polyethyleneglycol paraisooctypheny/ethers (Tritons's).

Propylene glycol mono- and di-fatty acid esters are also provided for in the paste formulations. These esters include, for example, propylene glycol dicaprylate; propylene glycol dilaurate, propylene glycol hydroxystearate, propylene glycol isostearate, propylene glycol laurate, propylene glycol ricinoleate, and propylene glycol stearate, most preferably propylene glycol caprylic-capric acid diester as is available under the Trade Name MIGLYOL 840. Other compounds which function as viscosity modifiers are those which contain both hydroxy and amino function groups. Such compounds include, for example, monoethanolamide, diethanolamine and triethanolamine. These compounds, as well as their use, are well known to a practitioner in the pharmaceutical and veterinary arts.

The amount of viscosity modifier varies from formulation to formulation and the determination of the amount required is well within the routine skill of a practitioner in the formulation art. Preferred is about 0.01 to about 20% of viscosity modifier, based upon total weight of the composition. An especially preferred amount is about 0.05 to about 5%, with about 0.1 to about 2% being most preferred.

In addition to the therapeutic agent, the viscosity modifier, and the carrier, the formulation can contain other inert ingredients such as antioxidants, preservatives, stabilizers or surfactants. These compounds are well known in the formulation art. Antioxidant such as an alpha tocopherol. ascorbic acid, ascrobvl palmitate, fumeric acid, malic acid, sodium ascorbate, sodium metabisulfate, n-propyl gallate, BHA (butylated hydroxy anisole), BHT (butylated hydroxy toluene) monothioglycerol and the like, may be added to the present formulation. The antioxidants are generally added to the formulation in amounts of from about 0.01% to about 2.0%, based upon total weight of the formulation. Preservatives such as the parabens (methylparaben and/or propylparaben) are suitably used in the formulation in amounts ranging from about 0.01 to about 2.0%. Other preservatives include benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, and the like.

Surfactants can also be added to help solublize the active drug, to prevent crystallization, and to prevent phase separation. Some examples of the surfactants are: glyceryl monooeleate, polyoxyethylene sorbitan fatty acid esters, sorbitan esters, polyvinyl alcohol, Pluronics, sodium lauryl sulfate, etc. Again, these compounds, as well as their amounts are well known in the art.

Absorbents may also be added to the paste formulation. Such compounds are well known in the art to the practitioner as well as their use in pastes. These compounds effectively prevents or alleviates the phase separation of the product during storage. Preferred absorbents include magnesium carbonate, calcium carbonate, starch, cellulose and its derivatives, or mixtures of absorbents with magnesium carbonate being especially preferred. The inclusion of these compounds is optional with amounts of 0% to about 30%, 0 to about 15% or about 1% to about 15% or about 1% to about 10%, based on total weight of the composition being especially preferred.

In some embodiments, the pharmaceutical composition of the present invention may be formulated for long-acting administration.

In some embodiments, the active agents may be used in conjunction with other treatments that use sustained-release formulations. A sustained-release matrix, in many instances, is a matrix made of materials, usually polymers, which are degradable by enzymatic or acid-based hydrolysis or by dissolution. Once inserted into the body, the matrix may be acted upon by enzymes and body fluids. Examples of sustained-release matrix materials include, without limitation, liposomes, polylactides (polylactic acid), polyglycolide (polymer of glycolic acid), polylactide co-glylide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids (e.g., phenylalanine, tyrosine, isoleucine), polynucleotides, polyvinyl propylene, polyvinylpyrrolidone and silicone. Illustrative biodegradable matrices include a polylactide matrix, a polyglycolide matrix and a polylactide co-glycolide (co-polymers of lactic acid and glycolic acid) matrix.

The compositions used in the disclosed methods of treatment may be administered through parenteral drug administration, for example, intravenous injection or infusion, intramuscular injection, or subcutaneous injection.

The compositions used in the disclosed methods of treatment may be administered orally and can be incorporated into a delivery device. For example, a delivery device may be coated with or comprise the compositions disclosed herein. In some cases, the delivery device is a syringe.

In some embodiments, the oral administration for a land animal can be incorporated into the daily animal feed or water. In some embodiments, the oral administration for an aquatic animal can be incorporated directly into the water supply in which the aquatic animal resides.

In some embodiments, an active agent of a composition described herein is packaged into a biological compartment. Biological compartments can include, but are not limited to, viruses (lentivirus, adenovirus), nanospheres, liposomes, quantum dots, nanoparticles, microparticles, nanocapsules, vesicles, polyethylene glycol particles, hydrogels, and micelles. For example, a biological compartment can comprise a liposome. A liposome can be a self-assembling structure comprising one or more lipid bilayers, each of which can comprise two monolayers containing oppositely oriented amphipathic lipid molecules. Amphipathic lipids can comprise a polar (hydrophilic) headgroup covalently linked to one or two or more non-polar (hydrophobic) acyl or alkyl chains. Energetically unfavorable contacts between the hydrophobic acyl chains and a surrounding aqueous medium induce amphipathic lipid molecules to arrange themselves such that polar headgroups can be oriented towards the bilayer's surface and acyl chains are oriented towards the interior of the bilayer, effectively shielding the acyl chains from contact with the aqueous environment. Non-limiting examples of amphipathic compounds used in liposomes include phosphoglycerides and sphingolipids, representative examples of which include phosphatidylcholine, phosphatidylethanolamine, phosphatidylserine, phosphatidylinositol, phosphatidic acid, phoasphatidylglycerol, palmitoyloleoyl phosphatidylcholine, lysophosphatidylcholine, lysophosphatidylethanolamine, dimyristoylphosphatidylcholine (DMPC), dipalmitoylphosphatidylcholine (DPPC), dioleoylphosphatidylcholine, di stearoylphosphatidylcholine (DSPC), dilinoleoylphosphatidylcholine and egg sphingomyelin, or any combination thereof.

In some embodiments, a biological compartment comprises a nanoparticle. In some cases, a nanoparticle comprises a diameter of from about 40 nanometers to about 1.5 micrometers, from about 50 nanometers to about 1.2 micrometers, from about 60 nanometers to about 1 micrometer, from about 70 nanometers to about 800 nanometers, from about 80 nanometers to about 600 nanometers, from about 90 nanometers to about 400 nanometers, from about 100 nanometers to about 200 nanometers. In some instances, as the size of the nanoparticle increases, the release rate can be slowed or prolonged and as the size of the nanoparticle decreases, the release rate can be increased. In some cases, the amount of albumin in the nanoparticles ranges from about 5% to about 85% albumin (v/v), from about 10% to about 80%, from about 15% to about 80%, from about 20% to about 70% albumin (v/v), from about 25% to about 60%, from about 30% to about 50%, or from about 35% to about 40% In some cases, a pharmaceutical composition comprises up to 30, 40, 50, 60, 70 or 80% or more of the nanoparticle. In some instances, active agents of the disclosure are bound to the surface of the nanoparticle.

The compositions comprising one or more active agents described herein may be administered to a subject in one or more doses. In some embodiments, a composition comprises one or more active agents. In some embodiments, a composition comprising one or more active agents is administered with one or more addition compositions, each comprising one or more additional active agents. In some instances, a subject is administered one dose of one active agent and another dose of another active agent. In some embodiments, one or more active agents is an antiviral agent.

In some embodiments, it may be necessary to wait until the drug has cleared from the system of the animal before slaughter, egg collection, milk collection, etc. It is appreciated by one skilled in the art of administration to animals the timing of clearance of various drug/drug combinations. Acyclovir, Valacyclovir, and Famciclovir clear through the kidneys of an average sized horse in less than 24 hours.

In various embodiments, the compositions containing the one or more active agents described herein are administered for prophylactic and/or therapeutic treatments. In some prophylactic applications, the compositions are administered to a subject at risk for exposure to a virus. In some prophylactic applications, the compositions are administered to a subject exposed to a virus. In some prophylactic applications, the compositions are administered to a subject prior to and after exposure to a virus. In some embodiments, prophylactic compositions prevent or reduce the risk of viral infection in a seronegative subject. In some prophylactic applications, compositions containing the one or more active agents described herein are administered to a subject susceptible to or otherwise at risk of a particular disease, disorder or condition. Such an amount is defined to be a prophylactically effective amount or dose. In this use, the precise amounts also depend on the subject's state of health, weight, and the like. When used in subjects, effective amounts for this use will depend on the severity and course of the disease, disorder or condition, previous therapy, the subject's health status and response to the drugs, and the judgment of the treating veterinarian. In some embodiments, the disease is an infectious disease caused by a virus.

In some therapeutic applications, the compositions are administered to a subject already suffering from a disease or condition, in an amount sufficient to cure or at least partially arrest at least one of the symptoms of the disease or condition. Amounts effective for this use depend on the severity and course of the disease or condition, previous therapy, the subject's health status, weight, and response to the drugs, and the judgment of the treating veterinarian. Therapeutically effective amounts are optionally determined by methods including, but not limited to, a dose escalation and/or dose ranging clinical trial. In some embodiments, the disease is an infectious disease caused by a virus. In some cases, the virus is herpesvirus.

In certain embodiments wherein the subject's condition does not improve, upon the veterinarian's discretion the administration of composition comprising the one or more active agents is administered chronically, that is, for an extended period of time, including throughout the duration of the subject's life in order to ameliorate or otherwise control or limit the symptoms of the subject's disease or condition.

In certain embodiments wherein a subject's status does improve, the dose of active agent(s) being administered is temporarily reduced or temporarily suspended for a certain length of time (i.e., a drug holiday). In some embodiments, the length of the drug holiday is between 2 days and 1 year, including by way of example only, 2 days, 3 days, 4 days, 5 days, 6 days, 7 days, days, 12 days, 15 days, 20 days, 28 days, or more than 28 days. The dose reduction during a drug holiday is, for example, by 10 A-100%, including only 10%, 15%, 20%, 25%, 30%, 35%, 40%, 45%, 50%, 55%, 60%, 65%, 70%, 75%, 80%, 85%0, 90%, 95%, and 100%.

Once improvement of the subject's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the subject requires intermittent treatment on a long-term basis upon any recurrence of symptoms.

In some embodiments, the dose of an active agent administered to a subject varies depending on factors such as time point during therapy, identity of active agent or combination of active agents, identity of disease, disease condition/severity, identity of subject (e.g., age, weight, sex), and route of administration.

In some embodiments, a composition described herein comprises a low dose of an active agent, such as an antiviral agent. In some embodiments, the low dose of the antiviral agent is from about 0.01 mg/kg to about 500 mg/kg. In some embodiments, the low dose of the antiviral agent is less than 0.01 mg/kg, or about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or 500 mg/kg. In some embodiments, the low dose of the antiviral agent is an amount similar to an amount of the antiviral agent useful for suppression treatment in a subject seropositive for the virus targeted by the antiviral agent.

In some embodiments, a composition described herein comprises a very high dose of an active agent, such as an antiviral agent. In some embodiments, the very high dose of the antiviral agent is from about 500 mg/kg to about 1000 mg/kg. In some embodiments, the very high dose of the antiviral agent is about 500 mg/kg to about 1000 mg/kg. In some cases, the very high dose of the antiviral agent is between about 500 mg/kg and about 1000 mg/kg, between about 600 mg/kg and about 1000 mg/kg, between about 700 mg/kg and about 1000 mg/kg, between about 800 mg/kg and about 1000 mg/kg, between about 900 mg/kg and about 1000 mg/kg, or greater than about 1000 mg/kg. In some embodiments, the very high dose of the antiviral agent is an amount greater than an amount of the antiviral agent useful for treatment of an outbreak in a seropositive subject infected with the virus targeted by the antiviral agent. In some embodiments, the antiviral agent is a herpesvirus antiviral agent.

In some embodiments, an active agent is administered in about a 0.01 mg/kg dosage. In some embodiments, an active agent is administered in about a 0.1 mg/kg dosage. In some embodiments, an active agent is administered in about a 1 mg/kg dosage. In some embodiments, an active agent is administered in about a 10 mg/kg dosage. In some embodiments, an active agent is administered in about a 20 mg/kg dosage. In some embodiments, an active agent is administered in about a 30 mg/kg dosage. In some embodiments, an active agent is administered in about a 40 mg/kg dosage. In some embodiments, an active agent is administered in about a 50 mg/kg dosage. In some embodiments, an active agent is administered in about a 100 mg/kg dosage. In some embodiments, an active agent is administered in about a 200 mg/kg dosage. In some embodiments, an active agent is administered in about a 300 mg/kg dosage. In some embodiments, an active agent is administered in about a 400 mg/kg dosage. In some embodiments, an active agent is administered in about a 500 mg/kg dosage. In some embodiments, an active agent is administered in about a 600 mg/kg dosage. In some embodiments, an active agent is administered in about a 700 mg/kg dosage. In some embodiments, an active agent is administered in about a 800 mg/kg dosage. In some embodiments, an active agent is administered in about a 900 mg/kg dosage. In some embodiments, an active agent is administered in about a 1,000 mg/kg dosage.

In some embodiments, the active agent is an antiviral agent. In some embodiments, the dosage is a very low dose. In some embodiments, the dosage is a very high dose. In some embodiments, the antiviral agent is an herpesvirus antiviral agent.

In some embodiments, a composition described herein comprises 1, 2, 3, 4, 5 or more active agents. In some embodiments, 1, 2, 3, 4 or 5 of the active agents are antiviral agents. In some embodiments, one antiviral agent is administered at a low dosage (as described above, e.g., from about 0.01 mg/kg to about 500 mg/kg) and one or more additional antiviral agents are administered at a second low dosage (as described above, e.g., from about 0.01 mg/kg to about 500 mg/kg). In some embodiments, one antiviral agent is administered at a low dosage (as described above, e.g., from about 0.01 mg/kg to about 500 mg/kg) and one or more additional antiviral agents are administered at a very high dosage (as described above, e.g., from about 500 mg/kg to about 1000 mg/kg).

In many implementations of the disclosure, the amount of active agent per dose is determined on a per body weight basis. For example, in an embodiment, the active agent is administered in an amount of about 0.01 mg/kg body weight to about 500 mg/kg body weight.

Those of skill will readily appreciate that dose levels often vary as a function of the specific active agent administered and the susceptibility of the subject to side effects. Preferred dosage forms of a given active agent are readily determinable by those of skill in the art. In some embodiments, the dosage or the amount of active agent in the dosage form are lower or higher than the ranges indicated herein, based on a number of variables in regard to an individual treatment regime. In various embodiments, the unit dosages are altered depending on a number of variables including, but not limited to, the activity of the agent, the mode of administration, the requirements of the individual subject, the condition of the subject (e.g., seronegative pre-exposure, seronegative post-exposure, seropositive), and/or the judgment of the practitioner.

In various embodiments, the dose of an active agent in a composition described herein is administered multiple times. The frequency of administration, in some instances, is dependent on the method of use, for example, for pre-exposure or post-exposure compositions. In some embodiments, an active agent is administered once per month, twice per month, three times per month, every other week, once per week, twice per week, three times per week, four times per week, five times per week, six times per week, every other day, daily, twice a day, three times a day, four times a day, five times a day, six times a day or more.

The duration of administration of the active agent (period of time over which the agent is administered), in many instances, varies depending on a number of factors. Examples of such factors include, without limitation, subject response, severity of symptoms, and disease type (e.g., virus type). In an example, an active agent is administered over a period of time of about one day to about one week, about one week to about two weeks, about two weeks to about four weeks, about one month to about two months, about two months to about four months, about four months to about six months, about six months to about eight months, about eight months to about 1 year, about 1 year to about 2 years or more.

In some embodiments, pharmaceutical compositions described herein are in unit dosage forms suitable for single administration of precise dosages. In some cases, in unit dosage form, the formulation is divided into unit doses containing appropriate quantities of one or more active agents. In some cases, the unit dosage is in the form of a package containing discrete quantities of the formulation. Non-limiting examples include packaged soft chews, tablets, or capsules, and gels or pastes in syringes. Aqueous suspension compositions can be packaged in single-dose non-reclosable containers. Multiple-dose reclosable containers can be used, for example, in combination with a preservative.

In some embodiments, an active agent described herein is present in a composition in a range of from about less than 0.01 mg/kg, or about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 53 mg/kg, 57 mg/kg, 60 mg/kg, 63 mg/kg, 67 mg/kg, 70 mg/kg, 73 mg/kg, 75 mg/kg, 77 mg/kg, 80 mg/kg, 83 mg/kg, 85 mg/kg, 87 mg/kg, 90 mg/kg, 93 mg/kg, 95 mg/kg, 97 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg·kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, or greater than 500 mg/kg. In some embodiments, the active agent is an antiviral agent. In some embodiments, the active agent is an herpesvirus antiviral agent.

In some applications, a herpesvirus prophylaxis and/or treatment kit is provided that includes an antiviral agent to be given to a subject in an effective amount such that herpesvirus replication is suppressed, herpesvirus activation is suppressed, or both; a delivery mechanism for the antiviral agent; dosage information specific to the antiviral agent; and use instructions specific to the delivery mechanism. In some instances, a kit can be used by a seronegative subject or a seropositive subject.

In some embodiments, the kit comprises a composition as described herein. In some embodiments, the composition is a pre-exposure composition. In some embodiments, the composition is a post-exposure composition. In some embodiments, the composition is a composition for suppression therapy. In some embodiments, the kit comprises one, two or more active agents. In some embodiments, the active agents are formulated together. In other embodiments, the active agents are formulated separately. In some embodiments, the kit comprises a means to administrate a composition comprising one or more active agents as described herein.

In some embodiments, the kit comprises suitable instructions in order to perform the methods of the kit. The instructions may provide information of performing any of the methods disclosed herein, whether or not the methods may be performed using only the reagents provided in the kit. The kit and instructions may require additional reagents or systems.

In some embodiments, a kit provided herein includes a carrier means being compartmentalized to receive in close confinement one or more containers such as vials, tubes, and the like, each of the containers comprising one of the separate elements to be used in a method provided herein.

The following examples are provided to further illustrate the advantages and features of the present disclosure, but are not intended to limit the scope of the disclosure. While they are typical of those that might be used, other procedures, methodologies, or techniques known to those skilled in the art may alternatively be used.

### EXAMPLES

### Example 1: Equivix for the Treatment and Prevention of Equine Herpesvirus Myeloencephalopathy (EHM)

The purpose of this study is to determine the safety and efficacy of Equivix (a novel combination of nucleoside analogs) in reducing the time and severity of Equine Myeloencephalopathy (EHM) in horses.

### Intervention Type:

Double blind placebo controlled study. Horses will be randomized in a 1:1 fashion.

### Treatment:

Equivix (40 mg/kg Valacyclovir+20 mg/kg Famciclovir) will be mixed with applesauce to form a paste for a final volume of 0.4 oz and administered via a syringe to horses ranging from 900-2000 lbs. Equivix will be administered every 8 hours for 7 days and then every 12 for 7 days for a total treatment of 14 days along with standard of care.

### Control Group:

Applesauce will be administered via a syringe every 8 hours for 7 days and then every 12 hours for 7 days for a total treatment of 14 days along with standard of care.

### Outcomes:

The primary outcome is reducing in the severity of ataxia and improvement in clinical scores. Secondary outcomes include reduction in viral shedding and viral viremia. The outcomes of clinical scores and severity of ataxia will be determined by a blinded veterinarian. Secondary outcomes will be determined by qPCR. Outcomes will be measured at baseline, day 7, and day 14.

### Species:

Equine

### Primary Field of Veterinary Medicine:

Infectious Disease

### Inclusion Criteria:

Horses within 2 days post-diagnosis of EHV-1 or horses with recent exposure to horses with a diagnosis of EHV-1.

### Exclusion Criteria:

Horses who are post 2 days diagnosis or who are already exhibiting neurological signs are ineligible for the study.

### Possible Benefits of Enrolling:

Currently, in California, there are outbreaks of EHV-1 that has led to neurological symptoms with several horses dying from the condition. Treatment for EHM is currently primarily supportive, including hydrating horses and administering anti-inflammatories. Antiviral treatment has the potential to reduce viremia, viral shedding, improve clinical outcomes and reduce the symptoms and severity of ataxia. Currently there are no recommended treatments for EHM. Equivix has the potential to save horses from a potentially lethal infection.

### Example 2—EHV Treatment Kit

An EHV prophylaxis kit for treatment of EHV. The kit has an oral formulation comprising a low dosage of valacyclovir (30 g or less) and a low dosage of famciclovir (15 g or less) and instructions having information on how to administer the composition. The instructions include dosing information useful for administration of the composition to an EHV seronegative subject to prevent EHV infection prior to EHV exposure. The EHV prophylaxis kit also includes dosing information and instructions for administration of the composition to an EHV seropositive subject to suppress EHV activation and viral shedding. The packing of the kit is designed to market to both EHV seronegative and EHV seropositive subjects.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

### REFERENCES

1. Grant, R., et al., Uptake of pre-exposure prophylaxis, sexual practices, and HIV incidence in men and transgender women who have sex with men: a cohort study. Lancet Infect Dis, 2014.
2. Center for Disease Control and Prevention. Preexposure prophylaxis for the prevention of HIV infection in the United States. 2014 Jun. 16, 2014; Available from: http://www.cdc.gov/hiv/pdf/guidelines/PrEPguidelines2014.pdf.
3. Food and Drug Administration. Truvada approved to reduce the risk of sexually transmitted HIV in people who are not infected by the virus. 2012; Available from: http://www.fda.gov/forconsumers/byaudience/forpatientadvocates/hivandaidsactivities/ucm3 12264.htm.
4. Grant, R., et al., Preexposure chemoprophylaxis for HIV prevention in men who have sex with men. N Engl J Med, 2010. 363(27): p. 2587-2599.
5. Grobskopf, L., et al., Randomized trial of clinical safety of daily oral tenofovir disoproxil fumarate among HIV-uninfected men who have sex with men in the United States. J Acquired Immune Defic Syndr, 2013. 64(1): p. 79-86.
6. Garré B, Gryspeerdt A, Croubels S, De Backer P, Nauwynck H. Evaluation of orally administered valacyclovir in experimentally EHV1-infected ponies. Veterinary Microbiology 2009; 135(3):214-221.
7. Thigpen, M., et al., Antiretroviral preexposure prophylaxis for heterosexual HIV transmission in Botswana. N Engl J Med, 2012. 367(5): p. 423-434.
8. Wald, A. and K. Link, Risk in human immunodeficiency virus (HIV) infection in herpes simplex virus type-2 (HSV-2) seropositive persons: a meta-analysis. J Infect Dis, 2002. 185: p. 45-52.
9. Freeman, E. and et. al, Herpes simplex virus 2 infection increases HIV acquisition in men and women: a systematic review and meta analysis of longitudinal studies. AIDS, 2006. 20: p. 73-83.
10. Reynolds, S., et al., Recent herpes simplex virus type 2 infection and the risk of human immunodeficiency virus type 1 acquistion in INdia. J Infect Dis, 2003. 187: p. 1513-21.
11. Zhu, J., et al., Persistence of HIV-1 receptor-positive cells after HSV-2 reactivation: a potential mechanism for increased HIV-1 acquisition. Nat Med, 2009. 15(8): p. 886-892.
12. Corey, L., et al., The effects of herpes simplex virus-2 on HIV-1 acquistion and transmission: a review of two overlapping epidemics. J Acquir Immune Defic Syndr, 2004. 35(5).
13. Maxwell, L., et al., Efficacy of the early administration of valacyclovir hydrochloride for the treatment of neuropathogenic equine herpesvirus type-1 infection in horses. Am J Net Res, 2018. 78(10): p. 1126-1139.
14. Lewis S, Renner E, Finno C. Evaluation of Aspirin and Valacyclovir Treatment and Vaccine Status in a Naturally Occurring Outbreak of EHV-1. AAEP Proceedings 2019; 65:509
15. Marchitello M, Sage A, White N, Estell K. Combined Treatment with Valacyclovir and Heparin Reduces Incidence Rate of EHM and Improves Survival in Naturally Occurring EHV-1 Outbreaks. AAEP Proceedings 2019; 65:502.
16. Huron, S., Cady, S., Pieloch, M. (2003). Compositions and Process for delivering an Additive. PCT Application No. PCT/US2003/025358.
17. Kluger, D., Hauler, A., Schmidt, C., Schroeckenfuchs, D., Waldron, N. (2021). Manufacturing of Semi-Plastic Pharmaceutical Dosage Units. U.S. patent Ser. No. 10/973,759B2.
18. Jun, C. (2001). Improved Paste Formulations. PCT Application No. PCT/EP2001/001155.

What is claimed is:

1. A pharmaceutical composition for treatment of herpesvirus infection, prophylaxis, or pre-exposure prophylaxis against herpesvirus infection in a subject in need thereof comprising: a therapeutically effective amount of (a) a first antiviral agent or a salt or solvate thereof and (b) a second antiviral agent or a salt or solvate thereof; and pharmaceutically acceptable excipient(s), diluent(s), carrier(s), and/or adjuvant(s).

2. The pharmaceutical composition of claim 1, comprising (a) and (b) in a dose ratio ((a):(b)) in mg/kg of about 5:1, about 4:1, about 3:1, about 2:1, about 1:1, about 1:2, about 1:3, about 1:4, or about 1:5.

3. The pharmaceutical composition of claim 2, comprising 0.01 mg/kg body weight to about 1000 mg/kg body weight of (a) and 0.01 mg/kg body weight to about 1000 mg/kg body weight of (b).

4. The pharmaceutical composition of claim 3, comprising about 1 mg/kg body weight to about 500 mg/kg body weight of (a).

5. The pharmaceutical composition of claim 3, comprising about 500 mg/kg body weight to about 1000 mg/kg body weight of (a).

6. The pharmaceutical composition of claim 3, comprising about 1 mg/kg body weight to about 500 mg/kg body weight of (b).

7. The pharmaceutical composition of claim 3, comprising about 500 mg/kg body weight to about 1000 my/kg body weight of (b).

8. The pharmaceutical composition of claim 2, formulated for oral administration.

9. The pharmaceutical composition of claim 8, wherein the oral formulation comprises a tablet, capsule, soft chew, a gel, a paste, a powder, a bait drop or a liquid.

10. The pharmaceutical composition of claim 2, formulated for parenteral administration.

11. The pharmaceutical composition of claim 10, wherein the parenteral administration comprises an intravenous injection or infusion, an intramuscular injection, or a subcutaneous injection.

12. The pharmaceutical composition of claim 11, wherein the parenteral administration comprises an intravenous injection or infusion.

13. The pharmaceutical composition of claim 2, formulated for administration once, twice, thrice, or four times daily.

14. The pharmaceutical composition of claim 2, formulated for long-acting administration.

15. The pharmaceutical composition of any one of claims 1-14, wherein (a) a first antiviral agent or a salt or solvate thereof comprises valacyclovir or a salt or solvate thereof, and wherein (b) a second antiviral agent or a salt or solvate thereof comprises famciclovir or a salt or solvate thereof.

16. The pharmaceutical composition of claim 15, additionally comprising a therapeutically effective amount of an additional antiviral agent.

17. The pharmaceutical composition of claim 16, wherein the additional antiviral agent comprises a helicase-primase inhibitor, amenamevir, N-methancocarbathymidine (N-MCT), tenofovir, emtricitabine, lamivudine, interfereon, ribavirin, boceprevir, telaprevir, simeprevir, sofosbuvir, ledipasvir, tenofovir, ombitasvir, paritaprevir, penciclovir, pritelivir, brincidofovir, cidofovir, ganciclovir, valganciclovir, valomaciclovir, or ritonavir, CSA.

18. The pharmaceutical composition of claim 1, wherein the herpesvirus is a member of the Equine herpesvirus, Gallid herpesvirus, or Bovine herpesvirus family of herpesviruses.

19. The pharmaceutical composition of claim 18, wherein the herpesvirus is a member of the Equine herpesvirus family of herpesviruses.

20. The pharmaceutical composition of claim 19, wherein the Equine herpesvirus comprises EHV-1, EHV-3, EHV-4, or EHV-5.

21. The pharmaceutical composition of claim 18, wherein the herpesvirus is a member of the Gallid herpesvirus family of herpesviruses.

22. The pharmaceutical composition of claim 21, wherein the Gallid herpesvirus comprises GaAHV1 or GaAHV2.

23. The pharmaceutical composition of claim 18, wherein the herpesvirus is a member of the Bovine family of herpesviruses.

24. The pharmaceutical composition of claim 23, wherein the Bovine herpesvirus comprises BoAHV1 or BoAHV5.

25. A pharmaceutical composition for treatment of Equine Herpesvirus (EHV), Gallid herpesvirus (GHV), or Bovine herpesvirus (BHV) infection, prophylaxis, or pre-exposure prophylaxis against EHV, GHV, or BHV infection in a subject in need thereof comprising: a therapeutically effective amount of greater than 0.5 mg/kg body weight of one or more antiviral agent(s).

26. The pharmaceutical composition of claim 25, wherein the amount of the one or more antiviral agent(s) is from greater than 1 mg/kg to about 1000 mg/kg body weight.

27. The pharmaceutical composition of claim 25, wherein the one or more antiviral agent(s) comprises valacyclovir or a salt or solvate thereof, famciclovir or a salt or solvate thereof, penciclovir or a salt or solvate thereof, acyclovir or a salt or solvate thereof, or any combination thereof.

28. The pharmaceutical composition of claim 27, which comprises valacyclovir or a salt or solvate thereof and famciclovir or a salt or solvate thereof.

29. The pharmaceutical composition of claim 28, comprising about 0.05 mg to about 1000 mg/kg body weight of valacyclovir or a salt or solvate thereof and about 0.05 mg/kg to about 1000 mg/kg bodyweight of famciclovir or a salt or solvate thereof.

30. The pharmaceutical composition of claim 27, which comprises valacyclovir or a salt or solvate thereof and acyclovir or a salt or solvate thereof.

31. The pharmaceutical composition of claim 30, comprising about 0.05 mg to about 1000 mg/kg body weight of valacyclovir or a salt or solvate thereof and about 0.05 mg/kg to about 1000 mg/kg bodyweight of acyclovir or a salt or solvate thereof.

32. The pharmaceutical composition of claim 27, which comprises valacyclovir or a salt or solvate thereof and penciclovir or a salt or solvate thereof.

33. The pharmaceutical composition of claim 32, comprising about 0.05 mg to about 1000 mg/kg body weight of valacyclovir or a salt or solvate thereof and about 0.05 mg/kg to about 1000 mg/kg bodyweight of penciclovir or a salt or solvate thereof.

34. The pharmaceutical composition of claim 27, formulated for oral administration.

35. The pharmaceutical composition of claim 34, comprising a tablet, capsule, soft chew, a gel, a paste, a powder, a bait drop, or a liquid.

36. The pharmaceutical composition of claim 27, formulated for parenteral administration.

37. The pharmaceutical composition of claim 36, wherein the parenteral administration comprises an intravenous injection or infusion, an intramuscular injection, or a subcutaneous injection.

38. The pharmaceutical composition of claim 37, wherein the parenteral administration comprises an intravenous injection or infusion.

39. The pharmaceutical composition of claim 27, formulated for administration once, twice, thrice, or four times daily.

40. The pharmaceutical composition of claim 27, formulated for long-acting administration.

41. The pharmaceutical composition of claim 27, additionally comprising a therapeutically effective amount of an additional antiviral agent.

42. The pharmaceutical composition of claim 41, wherein the additional antiviral agent comprises a helicase-primase inhibitor, amenamevir, N-methancocarbathymidine (N-MCT), tenofovir, emtricitabine, lamivudine, interfereon, ribavirin, boceprevir, telaprevir, simeprevir, sofosbuvir, ledipasvir, tenofovir, ombitasvir, paritaprevir, penciclovir, pritelivir, brincidofovir, cidofovir, ganciclovir, valganciclovir, valomaciclovir, or ritonavir.

43. The pharmaceutical composition of claim 25, wherein the herpesvirus is a member of the Equine herpesvirus, Gallid herpesvirus or Bovine herpesvirus family of herpesviruses.

44. The pharmaceutical composition of claim 43, wherein the herpesvirus comprises a member of the Equine herpesvirus family of herpesviruses.

45. The pharmaceutical composition of claim 44, wherein the Equine herpesvirus comprises EHV-1, EHV-3, EHV-4, or EHV-5.

46. The pharmaceutical composition of claim 43, wherein the herpesvirus comprises a member of the Gallid herpesvirus family of herpesviruses.

47. The pharmaceutical composition of claim 46, wherein the Gallid herpesvirus comprises GaAHV1 or GaAHV2.

48. The pharmaceutical composition of claim 43, wherein the herpesvirus comprises a member of the Bovine herpesvirus family of herpesvirus.

49. The pharmaceutical composition of claim 48, wherein the Bovine herpesvirus comprises BoAHV1 or BoAHV5.

50. A method of pre-exposure prophylaxis against EHV, GHV, or BHV infection in a herpesvirus seronegative subject in need thereof, or a method of treating a herpesvirus infection, or a method of suppressing a herpesvirus infection, or a method of treatment or prophylactic treatment of a disease or condition associated with EHV, GHV, or BHV infection, the method comprising administering to the subject daily a therapeutically effective amount of (a) a first antiviral or a salt or solvate thereof and (b) a second antiviral or a salt or solvate thereof; and pharmaceutically acceptable excipient(s), diluent(s), carrier(s) and/or adjuvant(s).

51. The method of claim 50, wherein the pharmaceutical composition comprises (a) and (b) in a dose ratio in mg/kg of about 5:1, about 4:1, about 3:1, about 2:1, about 1:1, about 1:2, about 1:3, about 1:4, or about 1:5.

52. The method of claim 51, wherein the pharmaceutical composition comprises 0.05 mg/kg body weight to about 1000 mg/kg body weight of (a) and 0.05 mg/kg body weight to about 1000 mg/kg body weight of (b).

53. The method of claim 50, wherein the disease or infection associated with EHV infection is Equine Myeloencephalopathy (EHM) or Equine Multinodal Pulmonary Fibrosis (EMPF).

54. The method of claim 50, wherein the disease or infection associated with GHV infection is infectious laryngotraceitis (ILT) or Marek's Disease.

55. The method of claim 50, wherein the disease or infection associated with BHV infection is bovine rhinotracheitis or bovine meningoencephalitis.

56. A method of pre-exposure prophylaxis against Equine Herpesvirus (EHV), Gallid Herpesvirus (GHV), or Bovine Herpesvirus (BHV) infection in a herpesvirus seronegative subject in need thereof, or a method of treating a herpesvirus infection, or a method of suppressing a herpesvirus infection, or a method of treatment or prophylactic treatment of a disease or condition associated with EHV, GHV, or BHV infection, the method comprising administering to the subject daily a pharmaceutical composition comprising a therapeutically effective amount of greater than 0.5 mg/kg body weight of an antiviral agent(s) comprising acyclovir or a salt or solvate thereof, or valacyclovir or a salt or solvate thereof, penciclovir or a salt or solvate thereof, famciclovir or a salt or solvate thereof, or combinations thereof, whereby the risk of EHV, GHVm or BHV infection in the subject is reduced.

57. The method of claim 56, wherein the pharmaceutical composition comprises about 0.5 mg/kg body weight to about 1000 mg/kg body weight of the antiviral agent(s).

58. The method of claim 56, wherein the antiviral agent(s) comprises valacyclovir or a salt or solvate thereof.

59. The method of claim 56, wherein the antiviral agent(s) comprises famciclovir or a salt or solvate thereof.

60. The method of claim 56, wherein the antiviral agent(s) comprises penciclovir or a salt or solvate thereof.

61. The method of claim 56, wherein the antiviral agent(s) comprises acyclovir or a salt or solvate thereof.

62. The method of claim 56, which comprises administering 0.5 mg/kg to about 1000 mg/kg body weight of a second antiviral agent.

63. The method of claim 62, wherein the antiviral agent(s) comprises valacyclovir or a salt or solvate thereof and famciclovir or a salt or solvate thereof.

64. The method of claim 62, wherein the antiviral agent(s) comprises valacyclovir or a salt or solvate thereof and acyclovir or a salt or solvate thereof.

65. The method of claim 62, wherein the antiviral agent(s) comprise valacyclovir or a salt or solvate thereof and penciclovir or a salt or solvate thereof.

66. The method of claim 63, which comprises administering about 0.05 mg/kg body weight to about 1000 mg/kg body weight of valacyclovir or a salt or solvate thereof and about 0.05 mg/kg body weight to about 1000 mg/kg body weight of famciclovir or a salt or solvate thereof.

67. The method of claim 66, wherein the administering is oral.

68. The method of claim 66, wherein the administering is parenteral.

69. The method of claim 68, wherein the parenteral administration is intravenous.

70. The method of claim 66, wherein the administering is once daily, twice daily, thrice daily, or four times daily.

71. The method of claim 66, wherein the administering begins after exposure to a subject that is seropositive for herpesvirus.

72. The method of claim 66, wherein the administering begins prior to exposure to a subject that is seropositive to herpesvirus.

73. The method of claim 66, comprising administering the pharmaceutical composition to the herpesvirus seropositive subject.

74. The method of claim 66, wherein the herpesvirus is a member of the Equine herpesvirus, Gallid herpesvirus, or Bovine herpesvirus family of herpesviruses.

75. The method of claim 74, wherein the herpesvirus comprises a member of the Equine herpesvirus family of herpesviruses.

76. The method of claim 75, wherein the Equine herpesvirus comprises EHV-1, EHV-3, EHV-4, or EHV-5.

77. The method of claim 74, wherein the herpesvirus comprises a member of the Gallid herpesvirus family of herpesviruses.

78. The method of claim 77, wherein the Gallid herpesvirus comprises GaAHV1 or GaAHV2.

79. The method of claim 74, wherein the herpesvirus comprises a member of the Bovine herpesvirus family of herpesviruses.

80. The method of claim 79, wherein the Bovine herpesvirus comprises BoAHV1 or BoAHV5.

81. The method of claim 66, wherein the disease or infection associated with EHV infection is Equine Myeloencephalopathy (EHM) or Equine Multinodal Pulmonary Fibrosis (EMPF).

82. The method of claim 66, wherein the disease or infection associated with GHV infection is infectious laryngotracheitis (ILT) or Marek's Disease.

83. The method of claim 66, wherein the disease or infection associated with BHV infection is bovine rhinotracheitis or bovine meningoencephalitis.
